(19) United States
(12) Patent Application Publication
(10) Pub. No.: US 2014/0377216 A1
(43) Pub. Date: Dec. 25, 2014

(54) ANTICANCER FUSION PROTEIN

(71) Applicant: ADAMED SP. Z.O.O., CZOSNOW K/WARSZAWY (PL)

(72) Inventors: Jerzy Szczepan Pieczykolan, Radecznica (PL); Sebastian Dominik Pawlak, Warszawa (PL); Bartlomiej Maciej Zerek, Dabrowa (PL); Piotr Kamil Rozga, Skierniewice (PL)

(21) Appl. No.: 14/367,681

(22) PCT Filed: Dec. 22, 2012
(86) PCT No.: PCT/IB2012/057657

§ 371 Date: Jun. 20, 2014

(30) Foreign Application Priority Data
Dec. 28, 2011 (PL) P.397595

(51) Int. Cl. C07K 14/525; C07K 7/08; C07K 14/435; C12N 9/16; C07K 14/005; C12N 7/00; C07K 14/195; C07K 14/32; C07K 14/47; C07K 14/46

(52) U.S. Cl. CPC C07K 14/525; C07K 14/47; C07K 7/08; C07K 14/43563; C07K 7/086; C07K 14/463; C07K 14/43572; C07K 14/005; C12N 7/00; C07K 14/195; C07K 14/32; C07K 14/461; C12N 9/16; C07K 2319/00; C12N 2760/16033; C12Y 301/04003
USPC 424/85.1; 530/351; 435/188

(57) ABSTRACT

A fusion protein comprising domain (a) which is a functional fragment of hTRAIL protein sequence, which fragment begins with an amino acid at a position not lower than hTRAIL95, or a homolog of said functional fragment having at least 70% sequence identity, preferably 85% identity and ending with the amino acid hTRAIL281; and at least one domain (b) which is a sequence of a cytolytic effector peptide forming pores in the cell membrane, wherein the sequence of domain (b) is attached at the C-terminus or N-terminus of domain (a). The fusion protein can be used for the treatment of cancer diseases.

The invention relates to the field of therapeutic fusion proteins, especially recombinant fusion proteins. More particularly, the invention relates to fusion proteins comprising the fragment of a sequence of the soluble human TRAIL protein and a sequence of a peptide forming pores in the cell or mitochondrial membrane, pharmaceutical compositions containing them, their use in therapy, especially as anticancer agents, and to polynucleotide sequences encoding the fusion proteins, expression vectors containing the polynucleotide sequences, and host cells containing these expression vectors.

TRAIL (Tumor Necrosis Factor-Related Apoptosis Inducing Ligand) protein, a is member of the cytokines family, also known as Apo2L (Apo2-ligand), is a potent activator of apoptosis in tumor cells and in cells infected by viruses. TRAIL is a ligand naturally occurring in the body. TRAIL protein, its amino acid sequence, coding DNA sequence SEQ. No.s and protein expression systems were disclosed for the first time in EP0835305A1.

TRAIL protein exerts its anticancer activity by binding to pro-apoptotic surface TRAIL receptors 1 and 2 (TRAIL-R1/R2) and subsequent activation of these receptors. These receptors, also known as DR4 and DR5 (death receptor 4 and death receptor 5), are members of the TNF receptor family and are overexpressed by different types of cancer cells. Activation of these receptors can induce external signaling pathway of suppressor gene p53-independent apoptosis, which by activated caspase-8 leads to the activation of executive caspases and thereby degradation of nucleic acids. Caspase-8 released upon TRAIL activation may also cause the release of truncated Bid protein, which is translocated to mitochondria, where it stimulates the release of cytochrome c, thus indirectly amplifying the apoptotic signal from death receptors.

TRAIL acts selectively on tumor cells, essentially without inducing apoptosis in healthy cells which show resistance to this protein. Therefore, the enormous potential of TRAIL was recognized as an anticancer agent which acts on a wide range of different types of cancers, including hematologic malignancies and solid tumors, while sparing normal cells and exerting potentially relatively little side effects.

TRAIL protein is a type II membrane protein having the length of 281 amino acids, and its extracellular region comprising amino acid residues 114-281 upon cleavage by proteases forms soluble sTRAIL molecule of 20 kDa size, which is also biologically active. Both forms, TRAIL and sTRAIL, are capable of triggering apoptosis via interaction with TRAIL receptors present on target cells. Strong antitumor activity and very low systemic toxicity of soluble part of TRAIL molecule was demonstrated using cell lines tests. Also, preliminary human clinical studies with recombinant human soluble TRAIL (rhTRAIL) having amino acid sequence corresponding to amino acids 114-281 of hTRAIL, known under the INN dulanermin, showed its good tolerance and absence of dose limiting toxicity. Toxic effects of recombinant TRAIL protein on liver cells reported up to now appear to be associated with the presence of modification, i.e. polyhistidine tags, while untagged TRAIL showed no systemic toxicity.

Fragments of TRAIL shorter than 114-281 are also able to bind with membrane death receptors and induce apoptosis via these receptors, for example, as recently reported in EP 1 688 498 for recombinant circularly permuted mutant of 122-281hTRAIL.

However, in further clinical trials on patients the actual effectiveness of TRAIL as a monotherapy proved to be low. Also problematic was primary or acquired resistance to TRAIL shown by many cancer cells (see for example WO2007/022214). Resistance may be due to various mechanisms and may be specific for a cancer type and/or patient-dependent (Thorburn A, Behbakht K, Ford H. TRAIL receptor-targeted therapeutics: resistance mechanisms and strategies to avoid them. Drug Resist Updat 2008; 11: 17-24). This resistance limits the usefulness of TRAIL as an anticancer agent. Although the mechanism of resistance to TRAIL has not been fully understood, it is believed that it may manifest itself at different levels of TRAIL-induced apoptosis pathway, ranging from the level of cell surface receptors to the executive caspases within the signaling pathway.

To overcome this low efficiency and the resistance of tumors to TRAIL, various combination therapies with radio- and chemotherapeutic agents were designed, which resulted in synergistic apoptotic effect (WO2009/002947; A. Almasan and A. Ashkenazi, Cytokine Growth Factor Reviews 14 (2003) 337-348; R K Srivastava, Neoplasis, Vol 3, No. 6, 2001, 535-546, Soria J C et al., J. Clin. Oncology, Vol 28, No. 9 (2010), p. 1527-1533). The use of rhTRAIL for cancer treatment in combination with selected conventional chemotherapeutic agents (paclitaxel, carboplatin) and monoclonal anti-VEGF antibodies are described in WO2009/140469. However, such a combination necessarily implies well-known deficiencies of conventional chemotherapy or radiotherapy. Prior art is silent, however, about any data suggesting abolishing of cell resistance to TRAIL obtained by fusing TRAIL protein with other proteins or fragments thereof.

Moreover, the problem connected with TRAIL therapy appeared to be its low stability and rapid elimination from the body after administration.

The effect of destruction of cancer cells and inhibition of tumor proliferation as a result of disintegration (discontinuity) of the cell membrane or mitochondrial membrane is known. There are also attempts to use substances with cytolytic effect capable of membrane disintegration both as an anti-cancer therapy and adjunct anti-cancer therapy.

Many natural and synthetic peptides and proteins having cytolytic activity are known. Cytolytic peptides are also described as pore-forming peptides or cytolysins. Interactions of pore forming peptides with the surface of the membrane may be based on nonspecific electrostatic interactions of the positively charged peptide with negatively charged surface of cell membrane.

These peptides are generally of cationic character, so that they are capable of electrostatic interactions with surfaces with predominantly negatively charged particles. Upon contact and interaction of a cytolytic peptide with lipids on the cell surface, and after penetration inside the cell with the lipids on the surface of the mitochondrial membrane, interruption of the continuity of the cell membrane occurs, followed by formation of small size transmembrane pores, by which leakage of the contents of the cytoplasm, including ions, outside the cell occurs, resulting in rapid and irreversible electrolyte imbalance in the cell, cell lysis and death.

The interactions of pore-forming peptides with the surface of the membrane may also include interactions with specific receptors present on the surface.

Known naturally occurring cytolytic peptides of bacterial, plant or mammalian origin capable of forming pores in the cell membrane are often called hemolysines, because they cause lysis of red blood cells and other eukaryotic cells. These toxins include cecropin A and B, aurein 1.2, citropin 1.1, defensin (HNP-2), lactoferricin B, tachyplesin, PR-39, cytolysins of Enterococcus faecalis, delta hemolysin, diphtheria toxin, cytolysin of Vibrio cholerae, toxin from Actinia equina, granulysin, lytic peptides from Streptococcus intermedius, lentiviral lytic peptides, leukotoxin of Actinobacillus actinomycetemcomitans, magainin, melittin, lymphotoxin, enkephalin, paradaxin, perforin (in particular the N-terminal fragment thereof), perfringolysin O (PFO/theta toxin) from Clostridium perfringens, and streptolysins. Their usefulness as medicaments is limited by their ability to cause hemolysis.

Natural cytolytic peptides are described, for example, in R. Smolarczyk et al., Post [symbol] py Hig. Med. Dośw., 2009; 63: 360-368

There are also known synthetic cytolytic pore-forming peptides. They are designed to be devoid of hemolytic properties, to be less immunogenic, or to have surfaces enabling high binding specificity to cellular targets such as for example VEGFR (vascular endothelial growth factor receptor) family receptors and the receptors of the EGFR (epidermal growth factor receptor) family. They are often hybrids of natural cytolytic peptides fragments, such as a hybrid of cecropin A fragment and magainin 2 CA (1-8) MA (1-12) fragment or a hybrid of cecropin A fragment and melittin CAMEL (CA (1-7) MEL (2-9)) fragment. There are also known synthetic cytolytic peptides D-K₄-L₂-R₉ and D-K₆-L₉, consisting of amino acids lysine, arginine and leucine, part of which is in the form of D-amino acids. There are also known synthetic chimeric peptides RGD-4C_{D}(KLAKLAK)₂, which contains the RGD motif binding with integrin αᵥβ₃ and an effector domain composed of D-amino acids KLAKLAKKLAKLAK, and PTD-5_{D}(KLAKLAK)₂ containing PTD-5 motif which allows penetration into the cells and an effector domain composed of D-amino acids KLAKLAKKLAKLAK (see, for e.g., R. Smolarczyk et al., Post [symbol] py Hig. Med. Dośw., 2009, 63: 360-368). Other well-known cytolytic synthetic peptides are described, for example, in Regen et al., Biochem. Biophys. Res. Commun. 159: 566-571, 1989.

The destruction of the membrane occurring after adhering of the peptide to the membrane may occur by the mechanism of “barrel staves” (barrel-stave model), the mechanism of a “doughnut-like shape” (toroidal-pore model) or a “carpet” mechanism (see, for e.g., R. Smolarczyk et al., Post [symbol] py Hig. Med. Dośw., 2009; 63: 360-368).

The mechanism of “barrel staves” is observed for amphipathic peptides with alpha-helical conformation having a length of at least 23 amino acids. For example, peptides which cause the destruction of the membrane by the mechanism of “barrel staves” are gramicidin A, alameticin, perforin, pilosulin, synthetic peptides with repeated KLAK motifs, cathelicidin, peptides isolated from Entamoeba histolytica, parasporins and cecropins. Peptides, which cause the destruction of the membrane by the “toroidal pore model” include, for example, melittin and magainin. For example, peptides which cause the destruction of the membrane by the “carpet” model are cecropins A and B.

Disintegration of cell membrane with formation of pores may be also caused by interaction of peptides of a high positive charge with negatively charged membrane components. Such properties show, among others, granulysins, analogs and derivatives of melittin, peptides comprising K(L)xR motif, tachyplesin, bombesin, magainin and viscotoxin.

The formation of pores in the membrane of the target cell may also be associated with enzymatic activity of peptides. The enzymatic activity of phospholipase A is shown, for example, by phospholipases with specific phospho-diesterase activity against phosphatidylcholine and sphingomyelin, hemolysins and cytolysins having nonspecific cytolytic activity, or hemolysins and cytolysins having cytolytic activity against biological membranes containing, for example, cholesterol. This type of enzymatic activity resulting in the formation of pores in the cell or mitochondrial membrane is exhibited by listeriolysin, equinatoxin, phospholipase PC-PLC and alpha-toxin from Clostridium perfringens.

There are also known conjugates and chimeras of pore-forming peptides with domains capable to target to tumor cells. For targeting, there are used antigens, carbohydrate moieties or growth factor receptors, overexpressed on the surface of tumor cells. Targeted delivery provides high levels of pore-forming peptide on the cell surface which is necessary for cytolytic activity.

The use of targeted pore forming actinoporins is described in Panchal R G. et al., Poreforming proteins and their application in biotechnology. Curr Pharm Biotechnol 2002, 3:99-115; Panchal R G: Novel therapeutic strategies to selectively kill cancer cells. Biochem Pharmacol 1998, 55:247-252 and in Hoskin D W, Ramamoorthy A: Studies on anticancer activities of antimicrobial peptides. Biochim Biophys Acta-Biomembr 2008, 1778:357-87.

It is also known that pore-forming peptides and proteins may be endowed with the ability to direct to the tumor associated antigens and receptors by means of appropriate genetic modification as well as by chemical joining to the suitable ligands or antibodies. Such modifications are described for d-endotoxin of Bacillus thuringiensis, equinatoxin II from Actinia equina, sticholysin I of Stichodactyla helianthus and diphtheria toxin of Corynebacterium diphtheriae (Soletti R C., Potentiation of anticancer-drug cytotoxicity by sea anemone pore-forming proteins in human glioblastoma cells. Anti-Cancer Drugs 2008, 19:517-525; Pederzolli C,: Biochemical and cytotoxic properties of conjugates of transferrin with equinatoxin-II, a cytolysin from a sea anemone. Bioconjugate Chem 1995, 6:166-173, van der Spek J C.: Fusion protein toxins based on diphtheria toxin: Selective targeting of growth factor receptors of eukaryotic cells. Appl Chimeric Genes Hybrid Proteins Pt B 2000, 327:239-249).

Also described is a fusion protein consisting of pore forming sticholysin toxin I and a monoclonal antibody directed against a tumor specific antigen C2, and its usefulness in the treatment in a colon cancer cell line model (Tejuca M. et al., Construction of an immunotoxin with the pore forming protein St1 and/or C5, a monoclonal antibody against a colon cancer cell line, Int. Immunopharmacol. 2004, 4:731-744). A number of fusion proteins comprising diphtheria toxin and interleukin-2 or EGF, and their potential to destroy the cell overexpressing the target receptors is also described (Murphy J R, van der Spek J C, Targeting diphtheria-toxin to growth-factor receptors, Semin Cancer Biol 1995, 6:259-267).

There is also known the use of cleavage sites recognized by specific proteases in fusion proteins molecules comprising cytolytic peptides in order to enable the release of effector proteins in the tumor environment and, consequently, their internalization into tumor cells. For example, Panchal R. et al. (Nat Biotechnol 1996, 14:852-856) disclosed alpha-hemolysins comprising in their sequence a cleavage site recognized by cathepsin B, which is activated by a protease present in the tumor environment.

There are also known modified proaerolysins (PA), inactive precursors of bacterial cytolytic pore-forming proteins, activated when cleaved by protease of prostate cancer cells (PSA) (Williams S. A. et al., JNCI J. Natl. Cancer Inst. (2007) 99 (5): 376-385).

U.S. Pat. No. 5,817,771B1 discloses conjugates, including fusion proteins, of pore-forming cytolytic peptides with an antibody or antigen as an element selectively binding on a tumor cell, and linkers enabling the selective activation of the cytolytic peptide in the tumor environment, such as, for example cleavage site recognized by enzymes such as proteases, in particular proteases overexpressed specifically in the tumor environment.

Barua et al. (Cancer Letters 293 (2010) 240-253) reported that prostate cancer cell lines resistant to TRAIL and insensitive to treatment with death receptor agonist antibodies DR4 and DR5 become sensitive to these antibodies after pre-treatment of these cells with synthetic cationic amphipathic lytic peptide KLA containing KLAK sequences.

The present invention provides fusion proteins with anti-cancer properties, which contain a domain derived from TRAIL and a domain of a cytolytic effector peptide with pore-forming properties against cell and/or mitochondrial membranes of mammalian cells.

Each of the two domains of the protein of the invention has different functions. Due to the presence of a domain derived from hTRAIL, proteins according to the invention are directed selectively to cancer cells, wherein the elements of the protein exert their effects. In particular, TRAIL domain after binding with a cell may exert its activity of triggering apoptosis, and the effector peptide the activity of forming pores in cell and/or mitochondrial membrane and causing lysis of the cancer cell.

Delivery of the protein of the invention into the tumor environment allows to minimize toxicity and side effects against healthy cells in the body, as well as reduction of the frequency of administration of a medicament. In addition, targeted therapy with the use of proteins according to the invention allows to avoid the problem of low efficiency of previously known nonspecific therapies based on the pores formation in the cell or mitochondrial membrane with the use of plant or bacterial toxins, caused by high toxicity and by the necessity of administering high doses.

It turned out that in many cases fusion proteins of the invention are more potent than soluble hTRAIL and its variants including the fragment of a sequence.

Until now, effector peptides used in the fusion protein of the invention have not been used in medicine as such because of unfavorable kinetics, rapid degradation by nonspecific proteases and accumulation in the body caused by lack of proper sequence of activation of pathways, which is necessary to enable the proper action of the effector peptide at target site. Incorporation of the effector peptides into the fusion protein allows their selective delivery to the site where their action is desirable. Furthermore, the attachment of the effector peptide increases the mass of protein, which results in prolonged half-life and increased retention of protein in the tumor and its enhanced efficiency.

Novel fusion proteins have also at least reduced or limited, or even substantially eliminated haemolytic activity compared to their individual natural cytolytic peptides.

Additionally, in many cases, novel fusion proteins also overcome natural or induced resistance to TRAIL. Most likely, overcoming the resistance is due to destabilization of the cell membrane potential as a result of the fusion proteins binding to lipids of cell or mitochondrial membrane and formation of pores, which causes leakage of divalent ions outside the cell. As a consequence of binding to the lipids of mitochondrial membrane, the release of cytochrome C, SMAC/Diablo protein and AIF factor into the cytoplasm occurs, which causes proapoptotic caspase activation in the affected cell. Degradation of the mitochondrial membranes leads also to the activation of caspase-9, resulting in the induction of apoptosis.

### DESCRIPTION OF FIGURES

FIG. 1 presents tumor volume changes (% of initial stage) in Cby.Cg-foxn1(nu)/J mice burdened with lung cancer A549 treated with fusion protein of the invention of Ex. 11^{a} compared to rhTRAIL114-281;

FIG. 2 presents tumor growth inhibition values (% TGI) in Cby.Cg-foxn1 (nu)/J mice burdened with lung cancer A549 treated with fusion protein of the invention of Ex. 11^{a} compared to rhTRAIL114-281;

FIG. 3 presents tumor volume changes (% of initial stage) in Crl:SHO-PrkdcscidHrhr mice burdened with lung cancer A549 treated with fusion protein of the invention of Ex. 11^{a} compared to rhTRAIL114-281;

FIG. 4 presents tumor growth inhibition values (% TGI) in Crl:SHO-PrkdcscidHrhr mice burdened with lung cancer A549 treated with fusion protein of the invention of Ex. 11^{a} compared to rhTRAIL114-281;

FIG. 5 presents tumor volume changes (% of initial stage) in Crl:SHO-PrkdcscidHrhr mice nu burdened with lung cancer NCI-H460-Luc2 treated with fusion protein of the invention of Ex. 11^{a} compared to rhTRAIL114-281;

FIG. 6 presents tumor growth inhibition values (% TGI) in Crl:SHO-PrkdcscidHrhr mice burdened with lung cancer NCI-H460-Luc2 treated with fusion protein of the invention of Ex. 11^{a} compared to rhTRAIL114-281;

FIG. 7 presents tumor volume changes (% of initial stage) in Cby.Cg-foxn1(nu)/J mice burdened with prostate cancer PC3 treated with fusion protein of the invention of Ex. 11^{a} compared to rhTRAIL114-281;

FIG. 8 presents tumor growth inhibition values (% TGI) in Cby.Cg-foxn1(nu)/J mice burdened with prostate cancer PC3 treated with fusion protein of the invention of Ex. 11^{a} compared to rhTRAIL114-281;

FIG. 9 presents tumor volume changes (% of initial stage) in Crl:SHO-PrkdcscidHrhr mice burdened with pancreas cancer PANC-1 treated with fusion protein of the invention of Ex. 11^{a} compared to rhTRAIL114-281;

FIG. 10 presents tumor growth inhibition values (% TGI) in Crl:SHO-PrkdcscidHrhr mice burdened with pancreas cancer PANC-1 treated with fusion protein of the invention of Ex. 11^{a} compared to rhTRAIL114-281;

FIG. 11 presents tumor volume changes (% of initial stage) in Crl:SHO-PrkdcscidHrhr mice burdened with colon cancer HCT116 treated with fusion protein of the invention of Ex. 11^{a} compared to rhTRAIL114-281;

FIG. 12 presents tumor growth inhibition values (% TGI) in Crl:SHO-PrkdcscidHrhr mice burdened with colon cancer HCT116 treated with fusion protein of the invention of Ex. 11^{a} compared to rhTRAIL114-281;

FIG. 13 presents tumor volume changes (% of initial stage) in Crl:SHO-PrkdcscidHrhr mice burdened with colon cancer SW620 treated with fusion protein of the invention of Ex. 16^{a} compared to rhTRAIL114-281;

FIG. 14 presents tumor growth inhibition values (% TGI) in Crl:SHO-PrkdcscidHrhr mice burdened with colon cancer SW620 treated with fusion protein of the invention of Ex. 16^{a} compared to rhTRAIL114-281;

FIG. 15 presents tumor volume changes (% of initial stage) in Crl:SHO-PrkdcscidHrhr mice burdened with colon cancer Colo205 treated with fusion protein of the invention of Ex. 16^{a} compared to rhTRAIL114-281;

FIG. 16 presents tumor growth inhibition values (% TGI) in Crl:SHO-PrkdcscidHrhr mice burdened with colon cancer Col205 treated with fusion protein of the invention of Ex. 16^{a} compared to rhTRAIL114-281;

FIG. 17 presents tumor volume changes (% of initial stage) in Crl:SHO-PrkdcscidHrhr mice burdened with uterine sarcoma MES-SA/Dx5 treated with fusion protein of the invention of Ex. 11^{a} compared to rhTRAIL114-281;

FIG. 18 presents tumor growth inhibition values (% TGI) in Crl:SHO-PrkdcscidHrhr mice burdened with uterine sarcoma MES-SA/Dx5 treated with fusion protein of the invention of Ex. 11^{a} compared to rhTRAIL114-281;

FIG. 19 presents tumor volume changes (% of initial stage) in Crl:SHO-PrkdcscidHrhr mice burdened with pancreatic carcinoma MIA Paca-2 treated with fusion protein of the invention of Ex. 16^{b} compared to rhTRAIL114-281;

FIG. 20 presents tumor growth inhibition values (% TGI) in Crl:SHO-PrkdcscidHrhr mice burdened with pancreatic carcinoma MIA Paca-2 treated with fusion protein of the invention of Ex. 16^{b} compared to rhTRAIL114-281;

FIG. 21 presents tumor volume changes (% of initial stage) in Crl:SHO-PrkdcscidHrhr mice burdened with colon cancer HCT116 treated with fusion protein of the invention of Ex. 16^{b} compared to rhTRAIL114-281;

FIG. 22 presents tumor growth inhibition values (% TGI) in Crl:SHO-PrkdcscidHrhr mice burdened with colon cancer HCT116 treated with fusion protein of the invention of Ex. 16^{b} compared to rhTRAIL114-281;

FIG. 23 presents tumor volume changes (% of initial stage) in Crl:SHO-PrkdcscidHrhr mice burdened with hepatocellular carcinoma HepG2 treated with fusion protein of the invention of Ex. 16^{b} compared to rhTRAIL114-281;

FIG. 24 presents tumor growth inhibition values (% TGI) in Crl:SHO-PrkdcscidHrhr mice burdened with hepatocellular carcinoma HepG2 treated with fusion protein of the invention of Ex. 16^{b} compared to rhTRAIL114-281;

FIG. 25 presents tumor volume changes (% of initial stage) in Crl:SHO-PrkdcscidHrhr mice burdened with hepatoma PLC/PRF/5 treated with fusion protein of the invention of Ex. 16^{b} compared to rhTRAIL114-281; and

FIG. 26 presents tumor growth inhibition values (% TGI) in Crl:SHO-PrkdcscidHrhr mice burdened with hepatoma PLC/PRF/5 treated with fusion protein of the invention of Ex. 16^{b} compared to rhTRAIL114-281.

### DETAILED DESCRIPTION OF THE INVENTION

The invention relates to a fusion protein comprising:
domain (a) which is a functional fragment of the sequence of soluble hTRAIL protein, which fragment begins with an amino acid at a position not lower than hTRAIL95 and ends with the amino acid hTRAIL281 or a homolog of said functional fragment having at least 70% sequence identity, preferably 85% identity, and
at least one domain (b) which is the sequence of a cytolytic effector peptide forming pores in the cell membrane,
wherein the sequence of the domain (b) is attached at the C-terminus and/or N-terminus of domain (a),

The term “peptide” in accordance with the invention should be understood as a molecule built from plurality of amino acids linked together by means of a peptide bond. Thus, the term “peptide” according to the invention includes oligopeptides, polypeptides and proteins.

In the present invention, the amino acid sequences of peptides will be presented in a conventional manner adopted in the art in the direction from N-terminus (N-end) of the peptide towards its C-terminus (C-end). Any sequence will thus have its N-terminus on the left side and C-terminus on the right side of its linear presentation.

The term “a functional soluble fragment of the sequence of soluble hTRAIL protein” should be understood as denoting any such fragment of soluble hTRAIL protein that is capable of inducing apoptotic signal in mammalian cells upon binding to its receptors on the surface of the cells.

It will be also appreciated by a skilled person that the existence of at least 70% or 85% homology of the TRAIL sequence is known in the art.

It should be understood that domain (b) of the effector peptide in the fusion protein of the invention is neither hTRAIL protein nor a part or fragment of hTRAIL protein.

The fusion protein of the invention incorporates at least one domain (b) of the effector peptide, attached at the C-terminus and/or or at the N-terminus of domain (a).

By sequence hTRAIL it is understood the known sequence of hTRAIL published in the GenBank database under accession number P505591 as well as in EP0835305A1 and presented in the Sequence Listing of the present invention as SEQ. No. 90.

In a particular embodiment, domain (a) is the fragment of TRAIL sequence, beginning with an amino acid from the range of hTRAIL95 to hTRAIL121, inclusive, and ending with the amino acid TRAIL 281.

In particular, domain (a) may be selected from the group consisting of sequences corresponding to hTRAIL95-281, hTRAIL114-281, hTRAIL115-281, hTRAIL119-281 and hTRAIL121-281. It will be evident to those skilled in the art that hTRAIL95-281, hTRAIL114-281, hTRAIL115-281, hTRAIL116-281, hTRAIL119-281 and hTRAIL121-281 represent a fragment of human TRAIL protein starting with amino acid denoted with the number 95, 114, 115, 116, 119 and 121, respectively, and ending with the last amino acid 281, in the known sequence of TRAIL.

In another particular embodiment, domain (a) is the homolog of a functional fragment of soluble TRAIL protein sequence beginning at amino acid position not lower than hTRAIL95 and ending at amino acid hTRAIL281, the sequence of which is at least in 70%, preferably in 85%, identical to original sequence.

In specific variants of this embodiment domain (a) is the homolog of a fragment selected from the group consisting of sequences corresponding to hTRAIL95-281, hTRAIL114-281, hTRAIL115-281, hTRAIL116-281, hTRAIL119-281 and hTRAIL121-281.

It should be understood that the homolog of a TRAIL fragment is a variation/modification of the amino acid sequence of this fragment, wherein at least one amino acid is changed, including 1 amino acid, 2 amino acids, 3 amino acids, 4 amino acids, 5 amino acids, 6 amino acids, and not more than 15% of amino acids, and wherein the fragment of a modified sequence has preserved functionality of the TRAIL sequence, i.e. the ability to bind to cell surface death receptors and induce apoptosis in mammalian cells. Modification of the amino acid sequence may include, for example, substitution, deletion and/or addition of amino acids.

Preferably, the homolog of TRAIL fragment having modified sequence shows modified affinity to the death receptors DR4 (TRAIL-R1) or DR5 (TRAIL-R2) in comparison with the native fragment of TRAIL.

The term “modified affinity” refers to increased affinity and/or affinity with altered receptor selectivity.

Preferably, the homolog of the fragment of TRAIL having modified sequence shows increased affinity to the death receptors DR4 and DR5 compared to native fragment of TRAIL.

Particularly preferably, the homolog of a fragment of TRAIL having modified sequence shows increased affinity to the death receptor DR5 in comparison with the death receptor DR4, i.e. increased selectivity DR5/DR4.

Also preferably, the homolog of a fragment of TRAIL having modified sequence shows an increased selectivity towards the death receptors DR4 and/or DR5 in relation to the affinity towards the receptors DR1 (TRAIL-R3) and/or DR2 (TRAIL-R4).

Modifications of TRAIL resulting in increased affinity and/or selectivity towards the death receptors DR4 and DR5 are known to those skilled in the art. For example, Tur V, van der Root A M, Reis C R, Szegezdi E, Cool R H, Samali A, Serrano L, Quax W J. DR4-selective tumor necrosis factor-related apoptosis-inducing ligand (TRAIL) variants obtained by structure-based design. J. Biol. Chem. 2008 Jul. 18; 283(29):20560-8, describe D218H mutation having increased selectivity towards DR4, and Gasparian M E, Chernyak B V, Dolgikh D A, Yagolovich A V, Popova E N, Sycheva A M, Moshkovskii S A, Kirpichnikov M P. Generation of new TRAIL mutants DR5-A and DR5-B with improved selectivity to death receptor 5, Apoptosis. 2009 June; 14(6):778-87, describe D269H mutation having reduced affinity towards DR4. hTRAIL mutants resulting in increased affinity towards one receptor selected from DR4 and DR5 compared with DR1 and DR2 receptors and increased affinity towards receptor DR5 compared with DR4 are also described in WO2009077857 and WO2009066174.

Suitable mutations are one or more mutations in the positions of native hTRAL selected from the group consisting of amino acids 131, 149, 159, 193, 199, 201, 204, 204, 212, 215, 218 and 251, in particular mutations involving substitution of an amino acid with a basic amino acid such as lysine, histidine or arginine, or an acidic amino acid such as glutamic acid or aspargic acid. In particular, one or more mutations selected from the group consisting of G131R, G131K, R1491, R149M, R149N, R149K, S159R, Q193H, Q193K, N199H, N199R, K201H, K201R, K204E, K204D, K204L, K204Y, K212R, S215E, S215H, S215K, S215D, D218Y, D218H, K251D, K251E and K251Q, as described in WO2009066174, may be mentioned.

Suitable mutations are also one or more mutations in the positions of native hTRAIL selected from the group consisting of amino acids 195, 269 and 214, particularly mutations involving substitution of an amino acid with abasic amino acid such as lysine, histidine or arginine. In particular, one or more mutations selected from the group consisting of D269H, E195R, and T214R, as described in WO2009077857, may be mentioned.

In a particular embodiment, the domain (a) which is the homolog of a fragment of hTRAIL, is selected from D218H mutant of the native TRAIL sequence, as described in WO2009066174, or the Y189N-R191K-Q193R-H264R-I266R-D269H mutant of the native TRAIL sequence, as described in Gasparian M E et al. Generation of new TRAIL mutants DR5-A and DR5-B with improved selectivity to death receptor 5, Apoptosis. 2009 June; 14(6): 778-87.

Domain (a), i.e. the fragment of TRAIL, is a domain responsible for binding of the construct of the fusion protein to death receptors on the surface of a cell. Furthermore, domain (a) upon binding will exert its known agonistic activity, i.e. activation of extrinsic pathway of apoptosis.

Domain (b) of the fusion protein of the invention is the domain of an effector peptide with cytolytic activity against eukaryotic cell.

In particular embodiments of the fusion protein of the invention, the effector peptide of domain (b) of the fusion protein is a peptide having pore-forming activity against cancer cells, selected from the group consisting of SEQ. No. 34 to SEQ. No. 56, and SEQ. No. 125 to SEQ. No. 132.

For the peptide with cytolytic activity it is meant a peptide having the ability of forming pores in the cell membrane, and after penetration into the cell, also in the mitochondrial membrane, thereby disrupting the continuity of the membrane. As a result of the disruption of membrane, a leakage of the contents of the cytoplasm, including ions, outside the cell occurs, which causes rapid and irreversible electrolyte imbalance in the cell, and its destruction (cell lysis).

The ability of a peptide to form pores in the cell or mitochondrial membrane and thus causing cell lysis a can be determined by a method of testing permeabilization of cell membranes known to those skilled in the art, for example by measuring the release from the cell of intracellular substances which previously had been applied to the cell, e.g. of ATP or radiolabelled marker, or by measuring the uptake of a dye, such as trypan blue, which does not occur when the cells are intact.

The cytolytic effector peptide of the invention may be either a natural peptide or a synthetic peptide.

Natural cytolytic pore-forming peptide may be bacterial exotoxin such as alpha-HL, perfringolysin, pneumolysin, streptolysin O, listeriolysin, Bacillus thuringensis toxin, parasporin of Bacillus thuringensis, lytic molecules from E. coli such as hemolysin or colicin.

Natural cytolytic pore-forming peptide may be also an eukaryotic peptide such as human granulysin, pilosulins family, including pilosulin 1 and pilosulin-5 from the venom of the Australian ants Myrmecia Pilosula, magainin such as magainin-2 from the skin of African frog Xenopus laevis, aurein 1.2 from the skin of African frog Litoria raniformis, citropin 1.1 from the skin of the tree frog Litoria citropa, melittin from the venom of the honey bee Apis mellifera, defensins, such as alpha-defensin and beta-defensin isolated from human cells, lactoferricins, such as lactoferricin B from cow's milk, tachyplesin from leukocytes of the crab Tachypleus tridentatus, cecropins A and B, or pleurocidins isolated from the Pleuronectes americanus.

Synthetic cytolytic pore-forming peptide may be known cytolytic peptide such as the hybryd of cecropin A fragment and magainin 2 fragment CA(1-8)MA(1-12), the hybryd of cecropin A fragment and fragment of melittin CAMEL (CA(1-7)MEL(2-9)), synthetic cytolytic peptides consisting of positively charge amino acids lysine, arginine and leucine, the part of which are in the form of D-amino acids such as D-K₄-L₂-R₉ and D-K₆-L₉, and peptides containing domain composed of D-amino acid motif KLAKLAK or repetitions thereof, for example (KLAKLAK)₂, synthetic hybrid peptides of two lytic peptides such as hybrids magainin-bombesin and cecropin-melittin, synthetic fusion peptides containing the synthetic cytolytic peptide and domain binding to the receptor present on the cell surface other than TRAIL receptor or domain that allows penetration into the cell, or lytic peptides based on amphipathic helix model consisting of KLLLK and KLLK series or modified and/or truncated peptides (preferably in the form of fusions with transduction or targeting domains) of mammalian origin.

The effector peptide of domain (b) of the fusion protein of the invention may be a peptide forming pores in the cell or mitochondrial membrane by direct interactions of the peptides having high positive charge with the negatively charged membrane.

Exemplary sequences of the effector peptide in this embodiment are designated as SEQ. No. 34 (an active form of human granulysin), SEQ. No. 35 (15-amino acids synthetic lytic peptide), SEQ. No. 38 (peptide from tachyplesin), SEQ. No. 39 (fusion peptide bombesin-magainin 2), SEQ. No. 40 (magainin-2), SEQ. No. 42 (26-amino acids hybrid peptide cecropin-melittin), SEQ. No. 53 (viscotoxin A3 (VtA3)), and SEQ. No. 56 (fusion peptide comprising an EGF inhibitor and synthetic lytic peptide), SEQ. No. 132 (melittin), SEQ. No. 129 and SEQ. No. 131 (fusion peptide comprising bombesin and truncated versions of BMAP27 (B27) or BMAP28 (B28), SEQ. No. 130 (17-amino acids synthetic peptide).

The effector peptide of domain (b) of the fusion protein of the invention may be a pore-forming peptide possessing amphipathic alpha-helixes conformation enabling interactions with biological membranes.

Exemplary sequences of the effector peptide in this embodiment are designated as SEQ. No. 36 (pilosulin-1), SEQ. No. 37 (pilosulin-5), SEQ. No. 41 (14-amino acids synthetic lytic peptide), SEQ. No. 43 (27-amino acids peptide FF/CAP-18), SEQ. No. 44 (BAMP-28 peptide), SEQ. No. 45 (the analogue of isoform C of lytic peptide from Entamoeba histolytica), SEQ. No. 46 (the analogue of isoform A of lytic peptide from Entamoeba histolytica), SEQ. No. 47 (the analogue of isoform B of lytic peptide from Entamoeba histolytica), SEQ. No. 48 (the fragment of HA2 domain of influenza virus hemagglutinin), SEQ. No. 54 (the active fragment of human perforin), SEQ. No. 55 (parasporin-2 from Bacillus thuringensis), SEQ. No. 125 synthetic fusion peptide with KLLK motif, SEQ. No. 126, and SEQ. No. 127 (pleurocidin analogues), SEQ. No. 128 synthetic peptide with KLLK motif.

The effector peptide of domain (b) of the fusion protein of the invention may be a pore-forming peptide with enzymatic activity selected from the group of phospholipases, hemolysins or cytolysins. Exemplary sequences of the effector peptide in this embodiment are designated as SEQ. No. 49 (N-terminal domain of alpha-toxin from Clostridium perfringens with phospholipase C activity), SEQ. No. 50 (Listeriolysin 0), SEQ. No. 51 (phospholipase PC-PLC), and SEQ. No. 52 (equinatoxin EqTx-II).

The effector peptide of domain (b) with pore-forming activity against eukaryotic cell may be a peptide which is an active form of human granulysin, belonging to so the saponin-like family, which exhibit strong binding ability to membrane lipids (P. M. Lydyard, A. Whelan, M. W. Fanger, “Krótkie wyklady: Immunologia”, Wydawnictwo Naukowe P W N 2001). Due to ability to bind to membranes granulysin is able to degrade mitochondrial membranes. This process leads to the release into the cytoplasm of cytochrome C, protein SMAC/Diablo and the AIF factor, which causes activation of apoptotic cascade and activation of caspase-9, also resulting in the induction of apoptosis. Granulysin also activates Bid proteins to the form tBid, which is directly involved in the formation of pores in the membranes of mitochondria (Zhang et al, The Journal of Immunology, 182: 6993-7000, 2009).

In particular, such an effector peptide is 83-amino acids peptide presented in the attached sequence listing as SEQ. No. 34.

Another effector peptide of domain (b) with pore-forming activity against eukaryotic cell may be a synthetic cytolytic peptide composed of leucine (L) and lysine (K) and structurally resembling natural lytic peptides from bee venom or a peptide from Ameba histolitica (Makovitzki, A., Suppression of Human Solid Tumor Growth in Mice by Intratumor and Systemic Inoculation of Histidine-Rich and pH-Dependent Host Defense-like Lytic Peptides, cancer Research, 2009). Due to the high content of said amino acids the peptide has strong positive charge allowing its selective interaction with membranes of tumor transformed cells and penetration into their structure with formation of pores according to the “barrel staves” mechanism.

In particular, such an effector peptide is 15-amino acids peptide presented in the attached sequence listing as SEQ. No. 35.

Another effector peptide of domain (b) with pore-forming activity against eukaryotic cell may be a peptide pilosulin-1, which is a cationic molecule derived from venom of the Australian ant Myrmecia Pilosula. Pilosulin 1 is a peptide with high content of lysine and arginine regularly repeated in a sequence. Due to the high content of these amino acids, the peptide has a strong positive charge allowing its selective interaction with membranes of cancer cells and formation of pores through the “barrel staves” mechanism (Kourie et al., Am J Physiol Cell Physiol, 278: 1063-1087, 2000).

In particular, such an effector peptide is 56-amino acids peptide presented the attached sequence listing as SEQ. No. 36.

Another effector peptide of domain (b) with pore-forming activity against eukaryotic cell may be peptide pilosulin-5, responsible for ionic interactions with the cell membrane resulting in the formation of pores, and as a consequence inhibition of tumor growth. Pilosulin 5 is the peptide belonging to the pilosulins family derived from the venom of Australian ant Myrmecia Pilosula. This peptide has in its structure cyclically repeated pattern of amino acids lysine, alanine, and aspartic acid, imparting a positive charge, which can potentiate interactions with tumor cells surface.

In particular, such an effector peptide is 100-amino acids peptide presented in the attached sequence listing as SEQ. No. 37.

Another effector peptide of domain (b) with pore-forming activity against eukaryotic cell may be tachyplesin, cationic peptide isolated from leukocytes of the crab Tridentatus Tachypleus. After penetration inside eukaryotic cells tachyplesin exhibits high affinity to mitochondrial membranes and causes their destabilization through the “barrel staves” mechanism and leakage from the mitochondria into the cytoplasm of factors such as cytochrome C, protein SMAC/DIABLO and AIF factor, which leads to cell death (Chen, Y., et al., RGD-Tachyplezin inhibits tumor growth. Cancer Res, 2001. 61(6): p. 2434-8; Ouyang, G. L., et al., Effects of tachyplesin on proliferation and differentiation of human hepatocellular carcinoma SMMC-7721 cells. World J Gastroenterol, 2002. 8(6): p. 1053-8.).

In particular, such an effector peptide is 17-amino acids peptide presented in the attached sequence listing as SEQ. No. 38.

Another effector peptide of domain (b) may be a fusion peptide bombesin-magainin with cytolytic activity against eukaryotic cell.

Magainins family consists of 21-27 amino acid polypeptides isolated from the skin of the African frog Xenopus laevis. The amphipathic alpha-helical structure of the peptides allows them to form pores in the cell membrane through the “barrel staves” mechanism (Matsuzaki et al, Biochim Biophys Acta, 1327:119-130, 1997). Bombesin, a 14-amino acids peptide with high positive charge isolated from the skin of frogs, belongs to the group of tumor-homing peptides, and as such exhibits high affinity to the surface of some types of solid tumors and blood cancers, which are characterized by a strong negative charge of the cell membrane (Moody et al, Peptides, 1983; volume 4: 683-686). Bombesin is able to bind to cellular receptors for neuromedin B, a close homologue of bombesin existing in the human body, which are highly expressed on the surface of tumor cells. This significantly increases the level of specificity of bombesin and its accumulation in the tissues occupied by the tumor, while minimizing systemic toxicity. Conjugates of toxic peptides with bombesin exhibit enhanced antitumor activity compared to the individual proteins (Huawei et al, Mol. Pharmaceutics, 2010; 2:586-596). Magainin is characterized by limited binding properties to tumor cells receptors and consequently its cytotoxicity is manifested only at high concentrations. It was shown that fusion peptide comprising magainin and bombesin allows to increase specificity and cytotoxicity against tumor cells (Liu S. et al., Enhancement of cytotoxicity of antimicrobial peptide magainin II in tumor cells by bombesin-targeted delivery. Acta Pharmacol Sin. 2011 January; 32(1):79-88).

In particular, such an effector peptide is 40-amino acids peptide presented in the attached sequence listing as SEQ. No. 39.

Another effector peptide of domain (b) may be also a fusion peptide comprising bombesin and truncated versions of BMAP27 (B27) or BMAP28 (B28) with cytolytic activity against eukaryotic cell. Such chimeric peptide reveals cytotoxic activity and decreased systemic toxicity (Cai H. et al., Selective apoptotic killing of solid and hematologic tumor cells by bombesin-targeted delivery of mitochondria-disrupting peptides, (viol. Pharmaceutics, 2010, 7(2), pp. 586-596).

In particular, such effector peptides are 31-amino acids peptide presented in the attached sequence listing as SEQ. No. 129 and 29 amino acids peptide presented in the attached sequence listing as SEQ. No. 131.

Another effector peptide of domain (b) may be magainin-2 peptide with cytolytic activity against eukaryotic cell, forming pores in cell and mitochondrial membrane. In particular, such an effector peptide is 23-amino acids peptide presented in the attached sequence listing as SEQ. No. 40.

Another cytolytic effector peptide of domain (b) with activity against eukaryotic cell may be a synthetic lytic peptide. Synthetic peptides of formula (KLAKKLA)ₙ (where n is a number of repetitions of the motif) as amphipathic and alpha-helical proteins after penetration into the cell are selectively accumulated in the negatively charged mitochondrial membrane, causing formation of pores and destabilization of electrostatic potential of mitochondria, thereby selectively eliminating cells of selected cancer cell lines (Javadpour et al, J Med Chem, 39:3107-13, 1996).

In particular, such an effector peptide is 14-amino acids peptide presented in the attached sequence listing as SEQ. No. 41.

Another effector peptide of domain (b) with cytolytic activity against eukaryotic cell may be an another synthetic lytic peptide which disintegrates the cell so membrane in a detergent-like manner. (Papo N, Shai Y. New lytic peptides based on the D,L-amphipathic helix motif preferentially kill tumor cells compared to normal cells. Biochemistry. 2003 Aug. 12; 42(31):9346-54).

In particular, such an effector peptide is 17-amino acids peptide presented in the attached sequence listing as SEQ. No. 128.

Another effector peptide of domain (b) with cytolytic activity against eukaryotic cell may be cecropin-melittin hybrid peptide, which causes formation of pores in cell membrane and consequently leads to inhibition of tumor growth. Cecropin A contains in its structure a large number of positively charged amino acids such as lysine, leucine, and alanine (Quellette, A., J., and Selsted, M., E., 1996, FASEB. J., 10(11), 1280-9), due to which forms pores in the membrane of eukaryotic cells. In addition, cecropin A has toxic activity, destroying the structure of cell cytoskeleton structure by destabilizing the structure of microtubules (Jaynes, J. et al., 1989, Peptide Res., 2 (2), 157-60).

Melittin is a peptide constructed of 25 amino acids, having high positive charge and alpha helical structure, and therefore strongly interacts with the membranes of tumor cells and forming pores by the “barrel stave” mechanism (Smolarczyk, R. et al Peptydy—nowa klasa leków przeciwnowotworowych, Post [symbol] py Hig and Med. Doświadczalnej, 2009, 63: 360.368). Additionally, melittin stimulates membrane enzyme phospholipase A2, responsible for decomposition of membrane phospholipids, which results in the release of fatty acids which are components of the lipid bilayer of the cell membrane. Synthetic chimeric peptide cecropin melittin obtained from genetic fusion of positively charged N-terminus of cecropin peptide with hydrophobic N-terminus of melittin peptide exhibits greater cytotoxic activity against target cells and is devoid of haemolytic activity characteristic for cecropin and melittin (Boman, H., G. et al (1989) FEBS Lett 259, 103-106; Andreu, D. et al (1992) FEBS Lett. 296, 190-194).

In particular, such an effector peptide is 26-amino acids peptide presented in the attached sequence listing as SEQ. No. 42.

Another effector peptide of domain (b) with pore forming activity is the peptide FF/CAP18 described by Isogai E. in “Antimicrobial and Lipopolysaccharide-Binding Activities of C-Terminal Domain of Human CAP18 Peptides to Genus Leptospira”, The Journal of Applied Research, Vol. 4, No. 1, 2004, 180-185). FF/CAP18 is the analogue of 27-amino acids C-terminal sequence of human cathelicidin hCAP18₁₀₉₋₁₃₅, which was modified by replacement of 2 amino acid residues with phenylalanines. FF/CAP18 has strongly cationic character, increased in relation to the native sequence due to incorporated modification, and strongly binds to eukaryotic cell membranes. Once bound to the surface of the membrane, FF/CAP18 forms channels and ionic pores, leading to destabilization of electrostatic balance of cells. In addition, after penetration inside the cell, the analog builds into the mitochondrial membrane to form ion is channels, thus destabilizing the electrostatic potential of mitochondria and leading to the release from mitochondria to cytosol factors such as cytochrome C, SMAC/Diablo or AIF factor, which initiates the process of apoptosis.

In particular, such an effector peptide is 27-amino acids peptide presented in the attached sequence listing as SEQ. No. 43.

Another effector peptide of domain (b) with pore forming activity is a peptide BAMP-28 with strong positive charge belonging to the cathelicidins family. This peptide is also the structural analogue of human histatins, a group of 12 peptides with a mass below 4 kDa produced by salivary glands cells and exhibiting antibacterial and antifungal properties (W. Kamysz et al., Histatyny—bialka liniowe bogate w histydyn [symbol] , Nowa Stomatologia 2004). The N-terminal domain of BAMP-28 peptide is strongly positively charged and is responsible for docking to the cell membrane, whereas the C-terminal part is responsible for the cytotoxic activity. (Hugosson, M., D. et al., 1994). Antibacterial peptides and mitochondrial presequences affect mitochondrial coupling, respiration and protein import. Eur. J. Biochem. 223:1027-1033.). The mechanism of BMAP-28 peptide activity is primarily based on the formation of pores in cell and mitochondrial membranes (A. Risso et al., BMAP-28, an Antibiotic Peptide of Innate Immunity, Induces Cell Death through Opening of the Mitochondrial Permeability Transition Pore, MOLECULAR AND CELLULAR BIOLOGY, March 2002, p. 1926-1935).

In particular, such an effector peptide is 27-amino acids peptide presented in the attached sequence listing as SEQ. No. 44.

Another effector peptide of domain (b) with pore forming activity is the analogue of isoform A of lytic peptide from Entamoeba histolytica responsible for accumulation on the cell surface resulting in pores formation, which in consequence leads to inhibition of tumor growth. Three isoforms A, B, and C of the peptides of Entamoeba histolytica have been identified, located in the granular cytoplasm of the parasite. These are 77-amino acids polypeptides stabilized by three sulfide bridges, containing in the secondary structure four amphipathic helices (Leippe, M et al EMBO J. 11, 3501-3506, 1992). These peptides have lytic properties against eukaryotic cells (Leippe, M. and Müller-Eberhard, H. J. Toxicology 87, 5-18, 1994). The third helix in the structure of these peptides has a length suitable for penetration of the cell membrane and formation of pores. Based on the amino acid sequences comprising only the third helix domain of all three isoforms, a series of analog synthetic peptides (A3, B3 and C3) has been constructed having the pore-forming and cytotoxic activity is against the human cancer cell lines and characterized with low hemolytic activity (Andrä et al., FEBS Letters 385: 96-100, 1996).

In particular, such an effector peptide is 24-amino acids peptide presented in the attached sequence listing as SEQ. No. 45.

Another effector peptide of domain (b) with pore forming activity against eukaryotic cell is an analogue of isoform B of lytic peptide from Entamoeba histolytica.

In particular, such an effector peptide is 24-amino acids peptide presented in the attached sequence listing as SEQ. No. 46.

Another effector peptide of domain (b) with pore forming activity against eukaryotic cell is an analogue of isoform C of lytic peptide from Entamoeba histolytica.

In particular, such an effector peptide is 24-amino acids peptide presented in the attached sequence listing as SEQ. No. 47.

Another effector peptide of domain (b) with pore forming activity against eukaryotic cell is a homologue of 20-amino acids N-terminal fragment, so called “fusion peptide”, of HA2 domain of influenza virus haemagglutinin, responsible for interaction of viral capsid with host cell membrane (intercalation) resulting in formation of pores in the cell membrane of the host. Haemagglutinin (HA) of the influenza virus is a homotrimeric glycoprotein responsible for fusion of viral capsid with host cell membrane. Two domains can be distinguished in the structure of the protein, HA1 responsible for receptor binding and H2 responsible for interactions with cell membrane. In the structure of HA2 domain only N-terminal part (20 amino acids), so-called “fusion peptide”, directly intercalates into the structure of the cell membrane (Duffer P et al., J Biol Chem 271:13417-13421, 1996). Structural analysis of fusion peptide homologues showed that its activity is associated with conformational change leading to the formation of amphipathic alpha helices, which are capable of endosome membrane perforation (Takahashi S., Biochemistry 29: 6257-6264, 1990). Therefore, derivatives of “fusion peptide” can be used as effective carriers of biologically active substance providing an efficient and quick “escape” from endosomes.

In particular, such an effector peptide is 12-amino acids peptide presented in the attached sequence listing as SEQ. No. 48.

Another effector peptide of domain (b) with pore forming activity against eukaryotic cell is N-terminal domain of alpha toxin from Clostridium perfringens with phospholipase C activity against phosphatidylcholine and sphingomyelin from cell membranes, allowing formation of pores. N-terminal domain of the alpha-toxin of Clostridium perfringens includes the active center of phospholipase C.

In particular, such an effector peptide is 247-amino acids peptide presented in the attached sequence listing as SEQ. No. 49.

Another effector peptide of domain (b) with pore forming activity against eukaryotic cell is a fragment of listeriolysin O, a cholesterol-dependent pore-forming peptide belonging to the group of hemolysins secreted by pathogen and activated after endosome environment acidification (Schnupf P, Portnoy D A. Listeriolysin O: a phagosome-specific lysin. Microbes Infect. 2007 August; 9(10): 1176-87. Epub 2007 May 7). It has been shown that listeriolysin O in the form of a fusion protein with targeting protein can specifically eliminate tumor cells (Bergeft S, Frost S, Lille H. Listeriolysin O as cytotoxic component of an immunotoxin. Protein Sci. 2009 June; 18(6):1210-20).

In particular, such an effector peptide is 468-amino acids peptide presented in the attached sequence listing as SEQ. No. 50.

Another effector peptide of domain (b) with pore forming activity against eukaryotic cell is an active fragment of phospholipase PC-PLC. Phospholipase PC-PLC is responsible for efficient lysis of vacuoles in primary endothelial cells and acts synergistically with listeriolysin in the lysis of primary and secondary vacuole. A substrate for PC-PLC is phosphatidylcholine (Smith G A, Marquis H, Jones S, Johnston N C, Portnoy D A, Goldfine H. The two distinct phospholipases C of Listeria monocytogenes have overlapping roles in escape from a vacuole and cell-to-cell spread. Infect Immun. 1995 November; 63(11):4231-7).

In particular, such an effector peptide is 288-amino acids peptide presented in the attached sequence listing as SEQ. No. 51.

Another effector peptide of domain (b) with pore forming activity against eukaryotic cell is equinatoxin protein. Equinatoxin EqTx-II is a pore-forming cytolysin isolated from Actinia equina anemone, characterized by a high, non-specific toxicity with respect to mammalian cells.

In particular, such an effector peptide is 179-amino acids peptide presented in the attached sequence listing as SEQ. No. 52.

Another effector peptide of domain (b) with pore forming activity against eukaryotic cell is viscotoxin A3 (VtA3). Viscotoxin A3 is one of thionins from mistletoe (Viscum album). Structurally it consists of a 46-amino acids chain with three disulfide bridges typical for the family (Coulon A. et al., Comparative membrane interaction study of viscotoxins A3, A2 and B from mistletoe (Viscum album) and connections with their structures. Biochem J. 2003 Aug. 15; 374(Pt 1):71-8). Toxic properties of viscotoxin on cancer cell lines are known (Tabiasco J. et al Mistletoe viscotoxins increase natural killer cell-mediated cytotoxicity. Eur J. Biochem. 2002 May; 269(10):2591-600). The exact molecular mechanism of action has not been described for VtA3. It is known, however, that it involves formation of ion channels and damage of membrane structures by permeabilization. A strong positive charge of the molecule favors binding to both the nucleic acids and phospholipids (Giudici M, Pascual R, de la Canal L, Pfüller K, Pfüller U, Villalaín J. Interaction of viscotoxins A3 and B with membrane model systems: implications to their mechanism of action. Biophys J. 2003 August; 85 (2):971-81).

In particular, such an effector peptide is 46-amino acids peptide presented in the attached sequence listing as SEQ. No. 53.

Another effector peptide of domain (b) with pore forming activity against eukaryotic cell is a peptide which is an active fragment of human perforin. The use of proteins of human origin which are “invisible” to the immune system, including human perforin, can solve the problem of limited clinical usefulness of protein chimeras containing toxins of bacterial, animal or plant origin because of generated strong immunogenicity (Frankel A E. Reducing the immune response to immunotoxin. Clin Cancer Res. 2004 Jan. 1; 10(1 Pt 1):13-5). N-terminal 34-amino acids fragment of human perforin forming nonspecifically pores in the cell membrane retains selective cytotoxic activity of the whole protein (Liu C C, Walsh C M, Young J D. Perforin: structure and function. Immunol Today. 1995 April; 16(4):194-201). A perforin fragment fused to an antibody targeting to cancer cells retains selective cytotoxic activity of the whole protein (Wan L. Expression, purification, and refolding of a novel immunotoxin containing humanized single-chain fragment variable antibody against CTLA4 and the N-terminal fragment of human perforin. Protein Expr. Purif. 2006 August; 48(2):307-13. Epub 2006 Mar. 9).

In particular, such an effector peptide is 33-amino acids peptide presented in the attached sequence listing as SEQ. No. 54.

Another effector peptide of domain (b) with pore-forming activity against eukaryotic cell is parasporin-2 from Bacillus thuringensis. Parasporins family comprises 13 different toxins belonging to subgroups PS1, PS2-PS3, PS4 (Ohba M. Parasporin, a new anticancer protein group from Bacillus thuringiensis. Anticancer Res. 2009 January; 29(1):427-33). Parasporin-2 exerts specificity against cancer cells (MOLT-4, Jurkat, HL60, HepG2, CACO-2) and exists in a form of 37-kDa protoxin activated by cutting off by proteinase K a portion of N and C-terminal fragments of respectively 51 and 36 amino acids. Key action of parasporin-2 consist of oligomerization within the cell membrane to form pores having a diameter of about 3 nm, resulting in increasing its permeability. Effects of parasporin-2 activity depend on the type of cell lines tested and include formation of so-called “blebbs” or bulges caused by the outflow of the cytoplasm from the cells and their lysis (HepG2 and NIH-3T3 cells) or formation of vacuole-like structures resulting in the burst of cells (MOLT-4) (Kitada S. Cytocidal actions of parasporin-2, an anti-tumor crystal toxin from Bacillus thuringiensis. J. Biol. Chem. 2006 Sep. 8; 281(36):26350-60). In addition, activity of parasporin-2 leads to the destruction of the structure of microtubules, actin filaments entanglement, fragmentation of mitochondria and endoplasmic reticulum (Akiba T. Crystal structure of the parasporin-2 Bacillus thuringiensis toxin that recognizes cancer cells. J. Mol. Biol. 2009 Feb. 13; 386(1):121-33).

In particular, such an effector peptide is 251-amino acids peptide presented in the attached sequence listing as SEQ. No. 55.

Another effector peptide of domain (b) with pore-forming activity against eukaryotic cell is a fusion protein comprising synthetic lytic peptide and peptide inhibitor of the EGF receptor on the cell surface. Binding of an inhibitor of EGFR on the cell surface allows location of the lytic peptide to the cell surface, and additionally inhibits localized intracellularly receptor tyrosine kinase. Inhibition of kinase activity results in the lack of cascade of biochemical signals leading to the release of the cytoplasmic Ca²⁺ ions and to activation of RAS signaling pathway leading to increased activity of glycolyse pathways, protein synthesis and thus causes decreased cell proliferation and limited tumor progression (Carpenter G, Cohen S., (May 1990). “Epidermal growth factor”. The Journal of Biological Chemistry 265 (14): 7709-12). Synthetic peptides comprising repeated leucine and lysine residues as amphipathic and helical proteins selectively eliminate selected cancer cell lines (Javadpour et al., J. Med. Chem., 39:3107-13, 1996).

In particular, such an effector peptide is 32-amino acids peptide presented in the attached sequence listing as SEQ. No. 56.

Fusion peptide presented in the attached sequence listing as SEQ. No. 56, comprising an EGF inhibitor and synthetic lytic peptide, is novel and has not been described before.

Another effector peptide of domain (b) with pore-forming activity against eukaryotic cell is a fusion protein comprising synthetic lytic peptide with KLLK motif and a peptide being antagonist of PDGF receptor on the cell surface. Binding of an inhibitor of PDGF on the cell surface allows location of the lytic peptide to the cell surface, and additionally binding affects cell proliferation and angiogenesis (Ostman A. et al., PDGF Receptors as Targets in Tumor Treatment, Adv. Cancer Res., 2007; 97:247-74.)

In particular, such an effector peptide is 39-amino acids peptide presented in the attached sequence listing as SEQ. No. 125.

Fusion peptide presented in the attached sequence listing as SEQ. No. 125 and a fusion variant of PDGF antagonist and synthetic lytic peptide of SEQ. No. 125 is novel and has not been described before.

Another effector peptide of domain (b) with pore-forming activity against eukaryotic cell is a protein being analogue of pleurocidin. Pleurocidins are cationic α-helical proteins interacting with cell membrane (Cole A M et al., Isolation and characterization of pleurocidin, an antimicrobial peptide in the skin secretions of winter flounder. J Biol Chem. 1997 May 2; 272(18):12008-13). Pleurocidin-like peptides are active against breast carcinoma cells, including drug-resistant and slow-growing breast cancer cells. (Hilchie A L et al., Pleurocidin-family cationic antimicrobial peptides are cytolytic for breast carcinoma cells and prevent growth of tumor xenografts. Breast Cancer Res. 2011 Oct. 24; 13(5):R102).

In particular, such effector peptides are 25-amino acids peptide presented in the attached sequence listing as SEQ. No. 126 and 26 amino acids peptide presented in the attached sequence listing as SEQ. No. 127.

Another effector peptide of domain (b) with pore-forming activity against eukaryotic cell is a B27 peptide—a truncated version of BMAP27 protein isolated form bovine myeloid cells belonging to cathelicidin family. (Donati M. et al., Activity of Cathelicidin Peptides against Chlamydia spp., Antimicrob Agents Chemother. 2005 March; 49(3): 1201-1202).

In particular, such effector peptides are 25-amino acids peptide presented in the attached sequence listing as SEQ. No. 130.

Upon binding to TRAIL receptors present on the surface of cancer cells, the fusion protein will exert a double effect. Domain (a), that is a functional fragment of TRAIL or its homolog with preserved functionality, will exert its known agonistic activity, i.e. binding to death receptors on the cell surface and activation of extrinsic pathway of apoptosis. The effector peptide of the domain (b) of the fusion protein will be able to potentially exert its action extra-cellularly or intracellularly in parallel to the activity of TRAIL domain.

In the fusion protein according to the invention, the antitumor TRAIL activity is potentiated by formation of pores in the cell or mitochondrial membrane resulting in disturbance of electrostatic charge of the cell, leakage of ions from the cytoplasm or destabilization of electrostatic potential mitochondria and release into the cytoplasm of factors such as cytochrome C, SMAC/DIABLO or factor AIF, which in turn activates internally induced apoptosis synergistic with the signal from the attachment of TRAIL to functional receptors of DR series.

The new fusion proteins also exhibit at least a reduced or limited, or even substantially eliminated haemolytic activity characteristic for the individual natural cytolytic peptides.

In one of the embodiments of the invention, domain (a) and domain (b) are linked by at least one domain (c) comprising the sequence of a cleavage site recognized by proteases present in the cell environment, especially in the tumor cell environment, e.g. such as metalloprotease, urokinase or furin.

Sequences recognized by protease may be selected from:
a sequence recognized by metalloprotease MMP Pro Leu Gly Leu Ala Gly Glu Pro/PLGLAGEP, or fragment thereof which with the last amino acid of the sequence to which is attached forms a sequence recognized by metalloprotease MMP,
a sequence recognized by urokinase uPA Arg Val Val Arg/RVVR, or fragment thereof, which with the last amino acid of the sequence to which is attached forms a sequence recognized by urokinase,
   and combinations thereof, or
a sequence recognized by furin Arg Gln Pro Arg/RQPR, Arg Gln Pro Arg Gly/RQPRG, Arg Lys Lys Arg/RKKR) or others atypical sequences recognized by furin disclosed by M. Gordon et al., in Inf. and Immun, 1995, 63, No. 1, p. 82-87, or native sequences recognized by furin Arg His Arg Gln Pro Arg Gly Trp Glu Gln Leu/RHRQPRGWEQL or HisArgGlnProArgGlyTrpGluGln/HRQPRGWEQ) or fragment thereof, which with the last amino acid of the sequence to which is attached forms a sequence recognized by furin.

In one of the embodiments of the invention, the protease cleavage site is a combination of the sequence recognized by metalloprotease MMP and/or a sequence recognized by urokinase uPA and/or a sequence recognized by furin located next to each other in any order.

Preferably, in one of the embodiments domain (c) is a sequence recognized by furin selected from Arg Val Val Arg Pro Leu Gly Leu Ala Gly/RVVRPLGLAG and Pro Leu Gly Leu Ala Gly Arg Val Val Arg/PLGLAGRVVR.

Proteases metalloprotease MMP, urokinase uPA and furin are overexpressed in the tumor environment. The presence of the sequence recognized by the protease enables the cleavage of domain (a) from domain (b), i.e. the release of the functional domain (b) and thus its accelerated activation.

Activation of the effector peptide-functional domain (b) after internalization of the fusion protein into the cell may occur nonspecifically by a cleavage of domain (a) from domain (b) of the fusion protein of the invention by lisosomal enzymes (non-specific proteases).

The presence of the protease cleavage site, by allowing quick release of the effector peptide, increases the chances of transporting the peptide to the place of its action as a result of cutting off from the hTRAIL fragment by means of protease overexpressed in the tumor environment before random degradation of the fusion protein by non-specific proteases occurs.

Additionally, a transporting domain (d) may be attached to domain (b) of the effector peptide of the fusion protein of the invention.

Domain (d) may be selected from the group consisting of:

(d1) polyhistidine sequence transporting through the cell membrane, consisting of 6, 7, 8, 9, 10 or 11 histidine residues (His/H); and
(d2) polyarginine sequence transporting through the cell membrane, consisting of 6, 7, 8, 9, 10 or 11 arginine residues (Arg/R),
(d3) PD4 transporting sequence (protein transduction domain 4) Tyr Ala Arg Ala Ala Ala Arg Gln Ala Arg Ala/YARAAARQARA,
(d4) a transporting sequence consisting of transferrin receptor binding sequence Thr His Arg Pro Pro Met Trp Ser Pro Val Trp Pro/THRPPMWSPVWP,
(d5) PD5 transporting sequence (protein transduction domain 5, TAT protein) Tyr Gly Arg Lys Lys Arg Arg Gln Arg Arg Arg/YGRKKRRQRRR,
or fragments thereof, which with the last amino acid of the sequence to which they are attached form sequences of transporting domains (d1) or (d2);
and
combinations thereof.

The combination of domains, e.g. (d1) and (d2), may comprise in particular the combination (d1)/(d2) and (d2)/(d1).

Furthermore, the combination of domains, e.g. (d1) and (d2), may include domains located next to each other and connected to one end of domain (b) and/or domains linked to different ends of domain (b).

It should be understood that in the case when the fusion protein has both the transporting domain (d) attached to domain (b) and domain (c) of the cleavage site between domains (a) and (b), then domain (c) is located in such a manner that after cleavage of the construct transporting domain (d) remains attached to domain (b). In other words, if the fusion protein contains both the transporting domain (d) and the cleavage site domain (c), then domain (d) is located between domain (b) and domain (c), or is located at the end of domain (b) opposite to the place of attachment of domain (d).

The invention comprises also a variant, wherein domain (d) is located between two (c) domains, that is the variant wherein after cleavage of the construct transporting domain, preferably the translocation domain, is not attached neither to the TRAIL domain nor to the effector peptide domain.

The invention does not comprise such a variant in which domain (d) is located between domain (c) and domain (a), that is the variant wherein after cleavage of the construct transporting domain remains attached to the TRAIL domain.

In another embodiment, between domain (a) and domain (b) there is additionally located domain (e) comprising a sequence suitable for attachment of a PEG molecule to the fusion protein (pegylation linker). Such a linker may be known sequence Ala Ser Gly Cys Gly Pro Glu Gly/ASGCGPEG or fragments thereof, which with the last amino acid of the sequence to which it is attached forms a sequence suitable for attachment of a PEG molecule. The pegylation so linker may be also selected from the group of the following:

Ala Ala Cys Ala Ala/AACAA,
Ser Gly Gly Cys Gly Gly Ser/SGGCGGS,
and
Ser Gly Cys Gly Ser/SGCGS,

or fragment thereof, which with the last amino acid of the sequence to which it is attached forms a sequence suitable for attachment of a PEG molecule

Preferably, the sequence of pegylation linker is Ala Ser Gly Cys Gly Pro Glu Gly/ASGCGPEG.

Apart from main functional elements of the fusion protein and the cleavage site domain(s), the fusion proteins of the invention may contain a neutral sequence/sequences of a flexible steric linker. Such steric linkers are well known and described in the literature. Their incorporation into the sequence of the fusion protein is intended to provide the correct folding of proteins produced by the process of its overexpression in the host cells. In particular, steric linker may be a glycine, glycine-serine or glycine-cysteine-alanine linker.

In particular, steric linker may be a combination of glycine and serine residues such as Gly Gly Gly Gly Ser/GGGGS or any fragment thereof acting as steric linker, for example a fragment Gly Gly Gly Ser/GGGS, Gly Gly Gly/GGG or Gly Gly Gly Gly/GGGG, Gly Gly Ser Gly Gly, Gly Gly Ser Gly/GGSG, Gly Ser Gly/GSG or Ser Gly Gly/SGG, or combinations thereof.

In other embodiment, the steric linker may be any combination of glycine, serine and alanine residues such as Ala Ser Gly Gly/ASGG or any fragment thereof acting as steric linker, for example Ala Ser Gly/ASG. It is also possible to use the combination of steric linkers, for example the sequence Gly Gly Gly Ser Gly/GGGGS or any fragment thereof acting as steric linker, for example the fragment Gly Gly Gly/GGG, with another fragment acting as steric linker. In such a case the steric linker may be a combination of glycine, serine and alanine residues such as Gly Gly Gly Ser Ala Ser Gly Gly/GGGSASGG, Gly Gly Ser Gly Gly Gly Ser Gly Gly Gly/GGSGGGSGGG, Gly Gly Ser Gly Gly Gly Gly Gly Ser/GGSGGGGGS or Gly Gly Gly Gly Gly Gly Ser/GGGGGGS. In still another embodiment, steric linker may be a combination of serine and histidine residues Ser His His Ser/SHHS or Ser His His Ala Ser/SHHAS.

In still another embodiment, the steric linker may be also selected from single amino acid residues such as single glycine or cysteine residue, in particular one or two up to four glycine or cysteine residues.

In another embodiment, the linker may also be formed by a fragment of steric linkers described above, which with the terminal amino acid of the sequence to which it is attached forms a steric linker sequence.

In another embodiment, the steric linker may promote the formation and stabilization of the structure of the trimer of the fusion protein of the invention, thus increasing its half-life in the blood circulation system and preventing from deassociation which may affect activity of the protein after administration into the blood circulation system. In this case the linker is a combination of cysteine and alanine, for example, a fragment Cys Cys Ala Ala Ala Ala Ala Cys/CAAACAAC or Cys Cys Ala Ala Ala Ala Ala Cys/CAACAAAC or fragments thereof, which is the terminal amino acid sequence to which it is attached and forms a steric linker sequence stabilising the trimer structure.

In addition, the steric linker may also be useful for activation of functional domain (b), occurring in a non-specific manner. Activation of domain (b) in a non-specific manner may be performed by cutting off the domain (a) from the domain (b) of the fusion protein according to the invention due to pH-dependent hydrolysis of the steric linker.

Particular embodiments of the fusion protein of the invention are fusion proteins comprising a pore-forming peptide selected from the group of peptides represented by:

SEQ. No. 34, SEQ. No. 35; SEQ. No. 36, SEQ. No. 37, SEQ. No. 38, SEQ. No. 39, SEQ. No. 40, SEQ. No. 41, SEQ. No. 42, SEQ. No. 43, SEQ. No. 44, SEQ. No. 45, SEQ. No. 46, SEQ. No. 47, SEQ. No. 48, SEQ. No. 49, SEQ. No. 50, SEQ. No. 51, SEQ. No. 52, SEQ. No. 53, SEQ. No. 54, SEQ. No. 55m SEQ. No. 56, SEQ. No. 125, SEQ. No. 126, SEQ. No. 127, SEQ. No. 128, SEQ. No. 129, SEQ. No. 130, SEQ. No. 131, and SEQ. No. 132.

A detailed description of the structure of representative fusion proteins mentioned above are shown in the Examples presented below.

In accordance with the present invention, by the fusion protein it is meant a single protein molecule containing two or more proteins or fragments thereof, covalently linked via peptide bond within their respective peptide chains, without additional chemical linkers.

The fusion protein can also be alternatively described as a protein construct or a chimeric protein. According to the present invention, the terms “construct” or “chimeric protein”, if used, should be understood as referring to the fusion protein as defined above.

For a person skilled in the art it will be apparent that the fusion protein thus defined can be synthesized by known methods of chemical synthesis of peptides and proteins.

The fusion protein can be synthesized by methods of chemical peptide synthesis, especially using the techniques of peptide synthesis in solid phase using suitable resins as carriers. Such techniques are conventional and known in the art, and described inter alia in the monographs, such as for example Bodanszky and Bodanszky, The Practice of Peptide Synthesis, 1984, Springer-Verlag, New York, Stewart et al., Solid Phase Peptide Synthesis, 2nd Edition, 1984, Pierce Chemical Company.

The fusion protein can be synthesized by the methods of chemical synthesis of peptides as a continuous protein. Alternatively, the individual fragments (domains) of protein may be synthesized separately and then combined together in one continuous peptide via a peptide bond, by condensation of the amino terminus of one peptide fragment from the carboxyl terminus of the second peptide. Such techniques are conventional and well known.

Preferably, however, the fusion protein of the invention is a recombinant protein, generated by methods of gene expression of a polynucleotide sequence encoding the fusion protein in host cells.

For verification of the structure of the resulting peptide known methods of the analysis of amino acid composition of peptides may be used, such as high resolution mass spectrometry technique to determine the molecular weight of the peptide. To confirm the peptide sequence, protein sequencers can also be used, which sequentially degrade the peptide and identify the sequence of amino acids.

A further aspect of the invention is a polynucleotide sequence, particularly DNA sequence, encoding the fusion protein as defined above.

Preferably, the polynucleotide sequence, particularly DNA, according to the invention, encoding the fusion protein as defined above, is a sequence optimized for expression in E. coli.

Another aspect of the invention is also an expression vector containing the polynucleotide sequence, particularly DNA sequence of the invention as defined above.

Another aspect of the invention is also a host cell comprising an expression vector as defined above.

A preferred host cell for expression of fusion proteins of the invention is an E. coli cell.

Methods for generation of recombinant proteins, including fusion proteins, are well known. In brief, this technique consists in generation of polynucleotide molecule, for example DNA molecule encoding the amino acid sequence of the target protein and directing the expression of the target protein in the host. Then, the target protein encoding polynucleotide molecule is incorporated into an appropriate expression vector, which ensures an efficient expression of the polypeptide. Recombinant expression vector is then introduced into host cells for transfection/transformation, and as a result a transformed host cell is produced. This is followed by a culture of transformed cells to overexpress the target protein, purification of obtained proteins, and optionally cutting off by cleavage the tag sequences used for expression or purification of the protein.

Suitable techniques of expression and purification are described, for example in the monograph Goeddel, Gene Expression Technology, Methods in Enzymology 185, Academic Press, San Diego, Calif. (1990), and A. Staron et al., Advances Mikrobiol., 2008, 47, 2, 1983-1995.

Cosmids, plasmids or modified viruses can be used as expression vectors for the introduction and replication of DNA sequences in host cells. Typically plasmids are used as expression vectors. Suitable plasmids are well known and commercially available.

Expression vector of the invention comprises a polynucleotide molecule encoding the fusion protein of the invention and the necessary regulatory sequences for transcription and translation of the coding sequence incorporated into a suitable host cell. Selection of regulatory sequences is dependent on the type of host cells and can be easily carried out by a person skilled in the art. Examples of such regulatory sequences are transcriptional promoter and enhancer or RNA polymerase binding sequence, ribosome binding sequence, containing the transcription initiation signal, inserted before the coding sequence, and transcription terminator sequence, inserted after the coding sequence. Moreover, depending on the host cell and the vector used, other sequences may be introduced into the expression vector, such as the origin of replication, additional DNA restriction sites, enhancers, and sequences allowing induction of transcription.

The expression vector will also comprise a marker gene sequence, which confers defined phenotype to the transformed cell and enables specific selection of transformed cells. Furthermore, the vector may also contain a second marker sequence which allows to distinguish cells transformed with recombinant plasmid containing inserted coding sequence of the target protein from those which have taken up the plasmid without insert. Most often, typical antibiotic resistance markers are used, however, any other reporter genes known in the field may be used, whose presence in a cell (in vivo) can be easily determined using autoradiography techniques, spectrophotometry or bio- and chemi-luminescence. For example, depending on the host cell, reporter genes such as β-galactosidase, β-glucuronidase, luciferase, chloramphenicol acetyltransferase or green fluorescent protein may be used.

Furthermore, the expression vector may contain signal sequence, transporting proteins to the appropriate cell compartment, e.g. periplasma, where folding is facilitated. Additionally a sequence encoding a label/tag, such as HisTag attached to the N-terminus or GST attached to the C-terminus, may be present, which facilitates subsequent purification of the protein produced using the principle of affinity, via affinity chromatography on a nickel column. Additional sequences that protect the protein against proteolytic degradation in the host cells, as well as sequences that increase its solubility may also be present.

Auxiliary element attached to the sequence of the target protein may block its activity, or be detrimental for another reason, such as for example due to toxicity. Such element must be removed, which may be accomplished by enzymatic or chemical cleavage. In particular, a six-histidine tag HisTag or other markers of this type attached to allow protein purification by affinity chromatography should be removed, because of its described effect on the liver toxicity of soluble TRAIL protein. Heterologous expression systems based on various well-known host cells may be used, including prokaryotic cells: bacterial, such as Escherichia coli or Bacillus subtilis, yeasts such as Saccharomyces cervisiae or Pichia pastoris, and eukaryotic cell lines (insect, mammalian, plant).

Preferably, due to the ease of culturing and genetic manipulation, and a large amount of obtained product, the E. coli expression system is used. Accordingly, the polynucleotide sequence containing the target sequence encoding the fusion protein of the invention will be optimized for expression in E. coli, i.e. it will contain in the coding sequence codons optimal for expression in E. coli, selected from the possible sequence variants known in the state of art. Furthermore, the expression vector will contain the above described elements suitable for E. coli attached to the coding sequence.

Accordingly, in a preferred embodiment of the invention a polynucleotide sequence comprising a sequence encoding a fusion protein of the invention, optimized for expression in E. coli is selected from the group of polynucleotide sequences consisting of:

SEQ. No. 57; SEQ. No. 58; SEQ. No. 59; SEQ. No. 60; SEQ. No. 61; SEQ. No. 62; SEQ. No. 63; SEQ. No. 64; SEQ. No. 65; SEQ. No. 66; SEQ. No. 67; SEQ. No. 68; SEQ. No. 69; SEQ. No. 70; SEQ. No. 71; SEQ. No. 72; SEQ. No. 73; SEQ. No. 74; SEQ. No. 75; SEQ. No. 76; SEQ. No. 77; SEQ. No. 78; SEQ. No. 79; SEQ. No. 80; SEQ. No. 81; SEQ. No. 82, SEQ. No. 83; SEQ. No. 84; SEQ. No. 85; SEQ. No. 86; SEQ. No. 87; SEQ. No. 88; SEQ. No. 89; SEQ. No. 108; SEQ. No. 109; SEQ. No. 110; SEQ. No. 111; SEQ. No. 112; SEQ. No. 113; SEQ. No. 114, SEQ. No. 115; SEQ. No. 116; SEQ. No. 117; SEQ. No. 118; SEQ. No. 119; SEQ. No. 120; SEQ. No. 121; SEQ. No. 122; SEQ. No. 123 and SEQ. No. 124, which encode fusion proteins having amino acid sequences corresponding to amino acid sequences selected from the group consisting of amino acid sequences, respectively:
SEQ. No. 1; SEQ. No. 2; SEQ. No, 3; SEQ. No. 4; SEQ. No. 5; SEQ. No. 6; SEQ. No. 7; SEQ. No. 8; SEQ. No. 9; SEQ. No. 10; SEQ. No. 11; SEQ. No. 12; SEQ. No. 13; SEQ. No. 14; SEQ. No. 15; SEQ. No. 16; SEQ. No. 17; SEQ. No. 18; SEQ. No. 19; SEQ. No. 20; SEQ. No. 21; SEQ. No. 22; SEQ. No. 23; SEQ. No. 24; SEQ. No. 25; SEQ. No. 26, SEQ. No. 27; SEQ. No. 28; SEQ. No. 29; SEQ. No. 30; SEQ. No. 31; SEQ. No. 32; SEQ. No. 33; SEQ. No. 91; SEQ. No. 92; SEQ. No. 93; SEQ. No. 94; SEQ. No. 95; SEQ. No. 96; SEQ. No. 97, SEQ. No. 98; SEQ. No. 99; SEQ. No. 100; SEQ. No. 101; SEQ. No. 102; SEQ. No. 103; SEQ. No. 104; SEQ. No. 105; SEQ. No. 106, and SEQ. No. 107.

In a preferred embodiment, the invention provides also an expression vector suitable for transformation of E. coli, comprising the polynucleotide sequence selected from the group of polynucleotide sequences SEQ. No. 57 to SEQ. No. 87 and SEQ. No. 108 to SEQ. No. 124 indicated above, as well as E. coli cell transformed with such an expression vector.

Transformation, i.e. introduction of a DNA sequence into bacterial host cells, particularly E. coli, is usually performed on the competent cells, prepared to take up the DNA for example by treatment with calcium ions at low temperature (4° C.), and then subjecting to the heat-shock (at 37-42° C.) or by electroporation. Such techniques are well known and are usually determined by the manufacturer of the expression system or are described in the literature and manuals for laboratory work, such as Maniatis et al., Molecular Cloning. Cold Spring Harbor, N.Y., 1982).

The procedure of overexpression of fusion proteins of the invention in E. coli is expression system will be further described below.

The invention also provides a pharmaceutical composition containing the fusion protein of the invention as defined above as an active ingredient and a suitable pharmaceutically acceptable carrier, diluent and conventional auxiliary components. The pharmaceutical composition will contain an effective amount of the fusion protein of the invention and pharmaceutically acceptable auxiliary components dissolved or dispersed in a carrier or diluent, and preferably will be in the form of a pharmaceutical composition formulated in a unit dosage form or formulation containing a plurality of doses. Pharmaceutical forms and methods of their formulation as well as other components, carriers and diluents are known to the skilled person and described in the literature. For example, they are described in the monograph Remington's Pharmaceutical Sciences, ed. 20, 2000, Mack Publishing Company, Easton, USA.

The terms “pharmaceutically acceptable carrier, diluent, and auxiliary ingredient” comprise any solvents, dispersion media, surfactants, antioxidants, stabilizers, preservatives (e.g. antibacterial agents, antifungal agents), isotonizing agents, known in the art. The pharmaceutical composition of the invention may contain various types of carriers, diluents and excipients, depending on the chosen route of administration and desired dosage form, such as liquid, solid and aerosol forms for oral, parenteral, inhaled, topical, and whether that selected form must be sterile for administration route such as by injection. The preferred route of administration of the pharmaceutical composition according to the invention is parenteral, including injection routes such as intravenous, intramuscular, subcutaneous, intraperitoneal, intratumoral, or by single or continuous intravenous infusions.

In one embodiment, the pharmaceutical composition of the invention may be administered by injection directly to the tumor. In another embodiment, the pharmaceutical composition of the invention may be administered intravenously. In yet another embodiment, the pharmaceutical composition of the invention can be administered subcutaneously or intraperitoneally. A pharmaceutical composition for parenteral administration may be a solution or dispersion in a pharmaceutically acceptable aqueous or non-aqueous medium, buffered to an appropriate pH and isoosmotic with body fluids, if necessary, and may also contain antioxidants, buffers, bacteriostatic agents and soluble substances, which make the composition compatible with the tissues or blood of recipient. Other components, which may included in the composition, are for example water, alcohols such as ethanol, polyols such as glycerol, propylene glycol, liquid polyethylene glycol, lipids such as triglycerides, vegetable oils, liposomes. Proper fluidity and the particles size of the substance may be provided by coating substances, such as lecithin, and surfactants, such as hydroxypropyl-cellulose, polysorbates, and the like.

Suitable isotonizing agents for liquid parenteral compositions are, for example, sugars such as glucose, and sodium chloride, and combinations thereof.

Alternatively, the pharmaceutical composition for administration by injection or infusion may be in a powder form, such as a lyophilized powder for reconstitution immediately prior to use in a suitable carrier such as, for example, sterile pyrogen-free water.

The pharmaceutical composition of the invention for parenteral administration may also have the form of nasal administration, including solutions, sprays or aerosols. Preferably, the form for intranasal administration will be an aqueous solution and will be isotonic or buffered o maintain the pH from about 5.5 to about 6.5, so as to maintain a character similar to nasal secretions. Moreover, it will contain preservatives or stabilizers, such as in the well-known intranasal preparations.

The composition may contain various antioxidants which delay oxidation of one or more components. Furthermore, in order to prevent the action of microorganisms, the composition may contain various antibacterial and antifungal agents, including, for example, and not limited to, parabens, chloro-butanol, thimerosal, sorbic acid, and similar known substances of this type. In general, the pharmaceutical composition of the invention can include, for example at least about 0.01 wt % of active ingredient. More particularly, the composition may contain the active ingredient in the amount from 1% to 75% by weight of the composition unit, or for example from 25% to 60% by weight, but not limited to the indicated values. The actual amount of the dose of the composition according to the present invention administered to patients, including man, will be determined by physical and physiological factors, such as body weight, severity of the condition, type of disease being treated, previous or concomitant therapeutic interventions, the patient and the route of administration. A suitable unit dose, the total dose and the concentration of active ingredient in the composition is to be determined by the treating physician.

The composition may for example be administered at a dose of about 1 microgram/kg of body weight to about 1000 mg/kg of body weight of the patient, for example in the range of 5 mg/kg of body weight to 100 mg/kg of body weight or in the range of 5 mg/kg of body weight to 500 mg/kg of body weight. The fusion protein and the compositions containing it exhibit anticancer or antitumor and can be used for the treatment of cancer diseases. The invention also provides the use of the fusion protein of the invention as defined above for treating cancer diseases in mammals, including humans. The invention also provides a method of treating neoplastic/cancer diseases in mammals, including humans, comprising administering to a subject in need of such treatment an anti-neoplastic/anticancer effective amount of the fusion protein of the invention as defined above, optionally in the form of appropriate pharmaceutical composition.

The fusion protein of the invention can be used for the treatment of hematologic malignancies such as leukaemia, granulomatosis, myeloma and other hematologic malignancies. The fusion protein can also be used for the treatment of solid tumors such as breast cancer, lung cancer, including non-small cell lung cancer, colon cancer, pancreatic cancer, ovarian cancer, bladder cancer, prostate cancer, kidney cancer, brain cancer, and the like. Appropriate route of administration of the fusion protein in the treatment of cancer will be in particular parenteral route, which consists in administering the fusion protein of the invention in the form of injections or infusions, in the composition and form appropriate for this administration route. The invention will be described in more detail in the following general procedures and examples of specific fusion proteins.

### General Procedure for Overexpression of the Fusion Protein

### Preparation of a Plasmid

Amino acid sequence of a target fusion protein was used as a template to generate a DNA sequence encoding it, comprising codons optimized for expression in Escherichia coli. Such a procedure allows to increase the efficiency of further step of target protein synthesis in Escherichia coli. Resulting nucleotide sequence was then automatically synthesized. Additionally, the cleavage sites of restriction enzymes NdeI (at the 5′-end of leading strand) and XhoI (at the 3′-end of leading strand) were added to the resulting gene encoding the target protein. These were used to clone the gene into the vector pET28a (Novagen). They may be also be used for cloning the gene encoding the protein to other vectors. Target protein expressed from this construct can be optionally equipped at the N-terminus with a polyhistidine tag (six histidines), preceded by a site recognized by thrombin, which subsequently serves to its purification via affinity chromatography. Some targets were expressed without any tag, in particular without histidine tag, and those were subsequently purified on SP Sepharose. The correctness of the resulting construct was confirmed firstly by restriction analysis of isolated plasmids using the enzymes NdeI and XhoI, followed by automatic sequencing of the entire reading frame of the target protein. The primers used for sequencing were complementary to the sequences of T7 promoter (5′-TAATACGACTCACTATAGG-3′) and T7 terminator (5% GCTAGTTATTGCTCAGCGG-3′) present in the vector. Resulting plasmid was used for overexpression of the target fusion protein in a commercial E. coli strain, which was transformed according to the manufacturers recommendations. Colonies obtained on the selection medium (LB agar, kanamycin 50 μg/ml, 1% glucose) were used for preparing an overnight culture in LB liquid medium supplemented with kanamycin (50 μg/ml) and 1% glucose. After about 15 h of growth in shaking incubator, the cultures were used to inoculate the appropriate culture.

### Overexpression and Purification of Fusion Proteins—General Procedure A

LB medium with kanamycin (30 μg/ml) and 100 μM zinc sulfate was inoculated with overnight culture. The culture was incubated at 37° C. until the optical density (OD) at 600 nm reached 0.60-0.80. Then IPTG was added to the final concentration in the range of 0.25-1 mM. After incubation (3.5-20 h) with shaking at 25° C. the culture was centrifuged for 25 min at 6,000 g. Bacterial pellets were resuspended in a buffer containing 50 mM KH₂PO₄, 0.5 M NaCl, 10 mM imidazole, pH 7.4. The suspension was sonicated on ice for 8 minutes (40% amplitude, 15-second pulse, 10 s interval). The resulting extract was clarified by centrifugation for 40 minutes at 20000 g, 4° C. Ni-Sepharose (GE Healthcare) resin was pre-treated by equilibration with buffer, which was used for preparation of the bacterial cells extract. The resin was then incubated overnight at 4° C. with the supernatant obtained after centrifugation of the extract. Then it was loaded into chromatography column and washed with 15 to 50 volumes of buffer 50 mM KH₂PO₄, 0.5 M NaCl, 20 mM imidazole, pH 7.4. The obtained protein was eluted from the column using imidazole gradient in 50 mM KH₂PO₄ buffer with 0.5 M NaCl, pH 7.4. Obtained fractions were analyzed by SDS-PAGE. Appropriate fractions were combined and dialyzed overnight at 4° C. against 50 mM Tris buffer, pH 7.2, 150 mM NaCl, 500 mM L-arginine, 0.1 mM ZnSO₄, 0.01% Tween 20, and at the same time Histag, if present, was cleaved with thrombin (1:50). After the cleavage, thrombin was separated from the target fusion protein expressed with His tag by purification using Benzamidine Sepharose™ resin. Purification of target fusion proteins expressed without Histag was performed on SP Sepharose. The purity of the product was analyzed by SDS-PAGE electrophoresis (Maniatis et al, Molecular Cloning. Cold Spring Harbor, N.Y., 1982).

### Overexpression and Purification of Fusion Proteins—General Procedure B

LB medium with kanamycin (30 μg/ml) and 100 μM zinc sulfate was inoculated with overnight culture. Cultures were incubated at 37° C. until optical density (OD) at 600 nm reached 0.60-0.80. Then IPTG was added to the final concentration in the range 0.5-1 mM. After 20 h incubation with shaking at 25° C. the culture was centrifuged for 25 min at 6000 g. Bacterial cells after overexpression were disrupted in a French Press in a buffer containing 50 mM KH₂PO₄, 0.5 M NaCl, 10 mM imidazole, 5 mM beta-mercaptoethanol, 0.5 mM PMSF (phenylmethylsulphonyl fluoride), pH 7.8. Resulting extract was clarified by centrifugation for 50 minutes at 8000 g. The Ni-Sepharose resin was incubated overnight with the obtained supernatant. Then the resin with bound protein was packed into the chromatography column. To wash-out the fractions containing non-binding proteins, the column was washed with 15 to 50 volumes of buffer 50 mM KH₂PO₄, 0.5 M NaCl, 10 mM imidazole, 5 mM beta-mercaptoethanol, 0.5 mM PMSF (phenylmethylsulphonyl fluoride), pH 7.8. Then, to wash-out the majority of proteins binding specifically with the bed, the column was washed with a buffer containing 50 mM KH₂PO₄, 0.5 M NaCl, 500 mM imidazole, 10% glycerol, 0.5 mM PMSF, pH 7.5. Obtained fractions were analyzed by SDS-PAGE (Maniatis et al, Molecular Cloning. Cold Spring Harbor, N.Y., 1982). The fractions containing the target protein were combined and, if the protein was expressed with histidine tag, cleaved with thrombin (1U per 4 mg of protein, 8 h at 16° C.) to remove polyhistidine tag. Then the fractions were dialyzed against formulation buffer (500 mM L-arginine, 50 mM Tris, 2.5 mM ZnSO₄, pH 7.4).

In this description Examples of proteins originally expressed with histidine tag that was subsequently removed are designated with superscript a) next to the Example number. Proteins that were originally expressed without histidine tag are designated with superscript b) next to the Example number.

### Characterization of Fusion Proteins by 2-D Electrophoresis

In order to further characterize obtained proteins and to select precisely chromatographic conditions, isoelectric points of the proteins were determined. For this purpose, two-dimensional electrophoresis (2-D) method was used, in two stages according to the following schedule.

### Step 1. Isoelectrofocusing of Proteins in a pH Gradient and Denaturing Conditions.

Protein preparations at concentrations of 1-2 mg/ml were precipitated by mixing in a 1:1 ratio with a precipitation solution containing 10% trichloroacetic acid and 0.07% beta-mercaptoethanol in acetone. The mixture was incubated for 30 min at −20° C. and then centrifuged for 25 min at 15,000 g and 4° C. The supernatant was removed and the pellet was washed twice with cold acetone with 0.07% beta-mercaptoethanol. Then the residues of acetone were evaporated until no detectable odour. The protein pellet was suspended in 250 ml of rehydration buffer 8M urea, 1% CHAPS, 15 mM DTT, 0.5% ampholyte (GE Healthcare) with a profile of pH 3-11 or 6-11, depending on the strip subsequently used. The protein solution was placed in a ceramic chamber for isoelectrofocusing, followed by 13 cm DryStrip (GE Healthcare) with appropriate pH profile (3-11 or 6-11). The whole was covered with a layer of mineral oil. The chambers were placed in the Ettan IPGphor III apparatus, where isoelectrofocusing was conducted according to the following program assigned to the dimensions of the strip and the pH profile:

16 h dehydration at 20° C.

### Focusing in the Electric Field at a Fixed pH Gradient

| Time | Voltage | |
|---|---|---|
| 1 h | 500 | V |
| 1 h | gradient 500-1000 | V |
| 2 h 30 min | gradient 1000-8000 | V |
| 30 min | 8000 | V |

Then, the strip containing the focused proteins was washed for 1 min in deionised water, stained with Coomassie Brilliant and then decolorized and archived as an image to mark the location of proteins. Discoloured strip was equilibrated 2×15 min with a buffer of the following composition: 50 mM Tris-HCl pH 8.8, 6M urea, 1% DTT, 2% SOS, 30% glycerol.

### Step 2. Separation in a Second Direction by SDS-PAGE.

The strip was placed over the 12.5% polyacrylamide gel containing a single well per standard size and then separation was performed in an apparatus for SOS-PAGE, at a voltage of 200V for 3 hours. The gel was stained with Coomassie so Brilliant then archived with the applied scale. Proteins were identified by determining its weight on the basis of the standard of size, and its IPI was read for the scale of 6-11 on the basis of the curves provided by the manufacturer (GE Healthcare) (ratio of pH to % of length of the strip from the end marked as anode) or a scale of 3-11 on the basis of the curve determined experimentally by means of isoelectrofocusing calibration kit (GE Healthcare).

### EXAMPLES

The representative examples of the fusion proteins of the invention are shown in the following Examples.

In the examples the amino acids sequences of fusion proteins are written from N-terminus to C-terminus of the protein. In the Examples, by TRAIL is always meant hTRAIL.

The following designations of amino acids sequences components are used, wherein next to the three-letter designation, the equivalent single-letter designation is given.

LINKER1: steric linker
Gly Gly/GG
LINKER2: steric linker
Gly Gly Gly/GGG
LINKER3: steric linker
Gly Ser Gly/GSG
LINKER4: steric linker
Gly Gly Gly Gly Ser/GGGGS
LINKER5: steric linker
Gly Gly Gly Gly Gly Ser/GGGGGS
LINKER6: steric linker
Gly Gly Ser Gly Gly/GGSGG
LINKER7: steric linker
Gly Gly Gly Ser Gly Gly Gly/GGGSGGG
LINKER8: steric linker
Gly Gly Gly Gly Ser Gly/GGGGSG
LINKER9: steric linker
Gly Gly Gly Ser Gly Gly Gly Gly Gly
Ser/GGGSGGGGGS
LINKER10: steric linker
Gly Gly Gly Gly Ser Gly Gly Gly Gly/GGGGSGGGG
LINKER11: steric linker
Gly Ser Gly Gly Gly Ser Gly Gly Gly/GSGGGSGGG
LINKER12: steric linker
Cys Ala Ala Cys Ala Ala Ata Cys/CAACAAAC
LINKER13: steric linker
Cys Ala Ala Ala Cys Ala Ala Cys/CAAACAAC
LINKER 14: steric linker
Cys/C
LINKER 15: steric linker
Gly/G
LINKER16: steric linker
Ser Gly Gly/SGG
FURIN: sequence cleaved by furin
Arg Lys Lys Arg/RKKR
FURIN. NAT: native sequence cleaved by furin
His Arg Gin Pro Arg Gly Trp Glu Gln/HRQPRGWEQ
UROKIN: sequence cleaved by urokinase
Arg Val Val Arg/RVVR
MMP: sequence cleaved by metalloprotease
Pro Leu Gly Leu Ala Gly/PLGLAG
PEG1: pegylation linker
Ala Ser Gly Cys Gly Pro Glu/ASGCGPE
PEG2: pegylation linker
Ala Ser Gly Cys Gly Pro Glu Gly/ASGCGPEG
TRANS1: transporting sequence
His His His His His His/HHHHHH
TRANS2: transporting sequence
Arg Arg Arg Arg Arg Arg Arg/RRRRRRR
TRANS3: transporting sequence
Arg Arg Arg Arg Arg Arg Arg Arg/RRRRRRRR
TRANS4: transporting sequence
Tyr Ala Arg Ala Ala Ala Arg Gln Ala Arg
Ala/YARAAARQARA
TRANS5: transporting sequence
Thr His Arg Pro Pro Met Trp Ser Pro Vat Trp
Pro/THRPPMWSPVWP
TRANS6: transporting sequence
Tyr Gly Arg Lys Lys Arg Arg Gln Arg Arg
Arg/YGRKKRRQRRR

### Example 1

### Fusion Protein of SEQ. No. 1

The protein of SEQ. No. 1 is a fusion protein having the length of 258 amino acids and the mass of 29.5 kDa, wherein domain (a) is the sequence of TRAIL121-281, and domain (b) of the effector peptide is the 83-amino acids active form of human granulysin (SEQ. No. 34) attached at the C-terminus of domain (a).

Additionally, between domain (a) and domain (b) there are sequentially incorporated steric linker G, steric linker (GSG), metalloprotease MMP cleavage site (PLGLAG) and urokinase cleavage site (RVVR). Thus, the structure of the fusion protein of the invention is as follows:

(SEQ. No. 34)
(TRAIL121-281)-LINKER15-LINKER3-MMP-UROKIN-

The amino acid sequence and the DNA encoding sequence comprising codons optimized for expression in E. coli are, respectively SEQ. No. 1 and SEQ. No. 57, as shown in the attached Sequence Listing.

The amino acid sequence SEQ. No. 1 of the structure described above was used as a template to generate its coding DNA sequence SEQ. No. 57. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 2

### Fusion Protein of SEQ. No. 2

The protein of SEQ. No. 2 is a fusion protein having the length of 261 amino acids and the mass of 30.09 kDa, wherein domain (a) is the sequence of TRAIL119-281, and domain (b) of the effector peptide is the 83-amino acids active form of human granulysin (SEQ. No. 34) attached at the N-terminus of domain (a).

Additionally, between domain (b) and domain (a) there are sequentially incorporated urokinase cleavage site (RVVR), metalloprotease cleavage site (PLGLAG) and steric linker (GGGGS). Thus, the structure of the fusion protein of the invention is as follows:

(SEQ. No. 34)
-UROKIN-MMP-LINKER4-(TRAIL119-281)

The amino acid sequence and the DNA encoding sequence comprising codons optimized for expression in E. coli are, respectively SEQ. No. 2 and SEQ. No. 58, as shown in the attached Sequence Listing.

The amino acid sequence SEQ. No. 2 of the structure described above was used as a template to generate its coding DNA sequence SEQ. No. 58. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed both with histidine tag (Ex. 2^{a}) and without histidine tag (Ex. 2^{b}).

### Example 3

### Fusion Protein of SEQ. No. 3

The protein of SEQ. No. 3 is a fusion protein having the length of 186 amino acids and the mass of 21.5 kDa, wherein domain (a) is the sequence of TRAIL121-281, and domain (b) of the effector peptide is the synthetic 15-amino acids lytic peptide (SEQ. No. 35) attached at the C-terminus of domain (a).

Additionally, between domain (a) and domain (b) there are sequentially incorporated metalloprotease cleavage site (PLGLAG) and urokinase cleavage site (RVVR). Thus, the structure of the fusion protein of the invention is as follows:

(SEQ. No. 35)
(TRAIL121-281)-MMP-UROKIN-

The amino acid sequence and the DNA encoding sequence comprising codons optimized for expression in E. coli are, respectively SEQ. No. 3 and SEQ. No. 59, as shown in the attached Sequence Listing.

The amino acid sequence SEQ. No. 3 of the structure described above was used as a template to generate its coding DNA sequence SEQ. No. 59. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 4

### Fusion Protein of SEQ. No. 4

The protein of SEQ. No. 4 is a fusion protein having the length of 227 amino acids and the mass of 25.7 kDa, wherein domain (a) is the sequence of TRAIL121-281, and domain (b) of the effector peptide is the 56-amino acids pilosulin-1 (SEQ. No. 36) attached at the N-terminus of domain (a).

Additionally, between domain (b) and domain (a) there are sequentially incorporated urokinase cleavage site (RVVR) and metalloprotease cleavage site (PLGLAG). Thus, the structure of the fusion protein of the invention is as follows:

(SEQ. No. 36)
-UROKIN-MMP-(TRAIL 121-281)

The amino acid sequence and the DNA encoding sequence comprising codons optimized for expression in E. coli are, respectively SEQ. No. 4 and SEQ. No. 60, as shown in the attached Sequence Listing.

The amino acid sequence SEQ. No. 4 of the structure described above was used so as a template to generate its coding DNA sequence SEQ. No. 60. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 5

### Fusion Protein of SEQ. No. 5

The protein of SEQ. No. 5 is a fusion protein having the length of 264 amino acids and the mass of 29.5 kDa, wherein domain (a) is the sequence of TRAIL95-281, and domain (b) of the effector peptide is the 56-amino acids pilosulin-1 (SEQ. No. 36), and is attached at the C-terminus of domain (a).

Additionally, between domain (a) and domain (b) there are sequentially incorporated steric linker (CAACAAC), steric linker (GGG), metalloprotease cleavage site (PLGLAG) and urokinase cleavage site (RVVR). Thus, the structure of the fusion protein of the invention is as follows:

(SEQ. No. 36)
(TRAIL95-281)-LINKER12-LINKER2-MMP-UROKIN-

The amino acid sequence and the DNA encoding sequence comprising codons optimized for expression in E. coli are, respectively SEQ. No. 5 and SEQ. No. 61 as shown in the attached Sequence Listing.

The amino acid sequence SEQ. No. 5 of the structure described above was used as a template to generate its coding DNA sequence SEQ. No. 61. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 6

### Fusion Protein of SEQ. No. 6

The protein of SEQ. No. 6 is a fusion protein having the length of 299 amino acids and the mass of 33.2 kDa, wherein domain (a) is the sequence of TRAIL95-281, and domain (b) of the effector peptide is the 90-amino acids peptide pilosulin 5 (SEQ. No. 37) attached at the C-terminus of domain (a).

Additionally, between domain (a) and domain (b) there are sequentially incorporated steric linker (GSG), steric linker (CAACAAAC), metalloprotease cleavage site (PLGLAG), urokinase cleavage site (RVVR) and steric linker (G). Thus, the structure of the fusion protein of the invention is as follows:

(SEQ. No. 37)
(TRAIL95-281)-LINKER3-LINKER12-MMP-UROKIN-
LINKER15-

The amino acid sequence and the DNA encoding sequence comprising codons optimized for expression in E. coli are, respectively SEQ. No. 6 and SEQ. No. 62 as shown in the attached Sequence Listing.

The amino acid sequence SEQ. No. 6 of the structure described above was used as a template to generate its coding DNA sequence SEQ. No. 62. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed both with histidine tag (Ex. 6^{a}) and without histidine tag (Ex. 6^{b}).

### Example 7

### Fusion Protein of SEQ. No. 7

The protein of SEQ. No. 7 is a fusion protein having the length of 224 amino acids and the mass of 25.6 kDa, wherein domain (a) is the sequence of TRAIL95-281, and domain (b) of the effector peptide is the 17-amino acids active peptide from tachyplesin (SEQ. No. 38) attached at the C-terminus of domain (a).

Additionally, between domain (a) and domain (b) there are sequentially incorporated steric linker (CAACAAAC), steric linker (GG), metalloprotease cleavage site (PLGLAG) and urokinase cleavage site (RVVR). Thus, the structure of the fusion protein of the invention is as follows

(SEQ. No. 38)
(TRAIL95-281)-LINKER12-LINKER1-MMP-UROKIN-

The amino acid sequence and the DNA encoding sequence comprising codons optimized for expression in E. coli are, respectively SEQ. No. 7 and SEQ. No. 63 as shown in the attached Sequence Listing.

The amino acid sequence SEQ. No. 7 of the structure described above was used as a template to generate its coding DNA sequence SEQ. No. 63. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed both with histidine tag (Ex. 7^{a}) and without histidine tag (Ex. 7^{b}).

### Example 8

### Fusion Protein of SEQ. No. 8

The protein of SEQ. No. 8 is a fusion protein having the length of 202 amino acids and the mass of 23.8 kDa, wherein domain (a) is the sequence of TRAIL 114-281, and domain (b) of the effector peptide is the 17-amino acids active peptide from tachyplesin (SEQ. No. 38) attached at the C-terminus of domain (a).

Additionally, between domain (a) and domain (b) there are sequentially incorporated metalloprotease cleavage site (PLGLAG), urokinase cleavage site (RVVR) and 7-arginine transporting sequence (RRRRRRR). Thus, the structure of the fusion protein of the invention is as follows:
(TRAIL114-281)-MMP-UROKIN-TRANS2-(SEQ. No. 38)

The amino acid sequence and the DNA encoding sequence comprising codons optimized for expression in E. coli are, respectively SEQ. No. 8 and SEQ. No. 64 as shown in the attached Sequence Listing.

The amino acid sequence SEQ. No. 8 of the structure described above was used as a template to generate its coding DNA sequence SEQ. No. 64. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed both with histidine tag (Ex. 8^{a}) and without histidine tag (Ex. 8^{b}).

### Example 9

### Fusion Protein of SEQ. No. 9

The protein of SEQ. No. 9 is a fusion protein having the length of 243 amino acids and the mass of 27.6 kDa, wherein domain (a) is the sequence of TRAIL 95-281, and domain (b) of the effector peptide is a 38-amino acids fusion peptide bombesin-magainin (SEQ. No. 39) attached at the N-terminus of domain (a).

Additionally, between domain (b) and domain (a) there are sequentially incorporated urokinase cleavage site (RVVR), metalloprotease cleavage site (PLGLAG) and steric linker (CAAACAAC). Thus, the structure of the fusion protein of the invention is as follows:

(SEQ. No. 39)
-UROKIN-MMP-LINKER13-(TRAIL95-281)

The amino acid sequence and the DNA encoding sequence comprising codons optimized for expression in E. coli are, respectively SEQ. No. 9 and SEQ. No. 65 as shown in the attached Sequence Listing.

The amino acid sequence SEQ. No. 9 of the structure described above was used as a template to generate its coding DNA sequence SEQ. No. 65. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 10

### Fusion Protein of SEQ. No. 10

The protein of SEQ. No. 10 is a fusion protein having the length of 196 amino acids and the mass of 22.4 kDa, wherein domain (a) is the sequence of TRAIL 119-281, and domain (b) of the effector peptide is the 23-amino acids magainin-2 (SEQ. No. 40) attached at the N-terminus of domain (a).

Additionally, between domain (b) and domain (a) there are sequentially incorporated urokinase cleavage site (RVVR) and metalloprotease cleavage site (PLGLAG). Thus, the structure of the fusion protein of the invention is as follows:

(SEQ. No. 40)
-UROKIN-MMP-(TRAIL119-281)

The amino acid sequence and the DNA encoding sequence comprising codons optimized for expression in E. coli are, respectively SEQ. No. 10 and SEQ. No. 66 as shown in the attached Sequence Listing.

The amino acid sequence SEQ. No. 10 of the structure described above was used as a template to generate its coding DNA sequence SEQ. No. 66. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 11

### Fusion Protein of SEQ. No. 11

The protein of SEQ. No. 11 is a fusion protein having the length of 202 amino acids and the mass of 23 kDa, wherein domain (a) is the sequence of TRAIL 121-281, and domain (b) of the effector peptide is the synthetic 14-amino acids lytic peptide (SEQ. No. 41) attached at the C-terminus of domain (a).

Additionally, between domain (a) and domain (b) there are sequentially incorporated steric linker (GGGGSGGGG), urokinase cleavage site (RVVR), metalloprotease cleavage site (PLGLAG) and 8-arginine transporting sequence (RRRRRRRR). Thus, the structure of the fusion protein of the invention is as follows:

(SEQ. No. 41)
(TRAIL121-281)-LINKER10-UROKIN-MMP-TRANS3-

The amino acid sequence and the DNA encoding sequence comprising codons optimized for expression in E. coli are, respectively SEQ. No. 11 and SEQ. No. 67 as shown in the attached Sequence Listing.

The amino acid sequence SEQ. No. 11 of the structure described above was used as a template to generate its coding DNA sequence SEQ. No. 67. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed both with histidine tag (Ex. 11^{a}) and without histidine tag (Ex. 11^{b}).

### Example 12

### Fusion Protein of SEQ. No. 12

The protein of SEQ. No. 12 is a fusion protein having the length of 205 amino acids and the mass of 23.2 kDa, wherein domain (a) is the sequence of TRAIL 121-281, and domain (b) of the effector peptide is the synthetic 14-amino acids lytic peptide (SEQ. No. 41) attached at the C-terminus of domain (a).

Additionally, between domain (a) and domain (b) there are sequentially incorporated steric linker (GG), sequence of pegylation linker (ASGCGPEG), steric linker sequence (GGG), metalloprotease cleavage site (PLGLAG), urokinase cleavage site (RVVR) and polyarginine transporting sequence (RRRRRRR). Thus, the structure of the fusion protein of the invention is as follows:

(SEQ. No. 41)
(TRAIL 121-281)-LINKER1-PEG2-LINKER2-MMP-UROKIN-
TRANS2-

The amino acid sequence and the DNA encoding sequence comprising codons optimized for expression in E. coli are, respectively SEQ. No. 12 and SEQ. No. 68 as shown in the attached Sequence Listing.

The amino acid sequence SEQ. No. 12 of the structure described above was used as a template to generate its coding DNA sequence SEQ. No. 68. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 13

### Fusion Protein of SEQ. No. 13

The protein of SEQ. No. 13 is a fusion protein having the length of 228 amino acids and the mass of 25.9 kDa, wherein domain (a) is the sequence of TRAIL 95-281, and domain (b) of the effector peptide is the 14-amino acids lytic peptide (SEQ. No. 41) attached at the C-terminus of domain (a).

Additionally, between domain (a) and domain (b) there are sequentially incorporated steric linker (GGGGSGGGG), urokinase cleavage site (RWR), metalloprotease cleavage site (PLGLAG) and 8-arginine transporting sequence (RRRRRRRR). Thus, the structure of the fusion protein of the invention is as follows:

(SEQ. No. 41)
(TRAIL 95-281)-LINKER10-UROKIN-MMP-TRANS3-

The amino acid sequence and the DNA encoding sequence comprising codons optimized for expression in E. coli are, respectively SEQ. No. 13 and SEQ. No. 69 as shown in the attached Sequence Listing.

The amino acid sequence SEQ. No. 13 of the structure described above was used as a template to generate its coding DNA sequence SEQ. No. 69. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed both with histidine tag (Ex. 13^{a}) and without histidine tag (Ex. 13^{b}).

### Example 14

### Fusion Protein of SEQ. No. 14

The protein of SEQ. No. 14 is a fusion protein having the length of 192 amino acids and the mass of 22.1 kDa, wherein domain (a) is the sequence of TRAIL 121-281, and domain (b) of the effector peptide is the synthetic 14-amino acids lytic peptide (SEQ. No. 41) attached at the N-terminus of domain (a).

Additionally, between domain (b) and domain (a) there are sequentially incorporated steric linker (C), urokinase cleavage site (RVVR) and metalloprotease cleavage site (PLGLAG). Additionally at the N-terminus of effector peptide is attached polyhistidine transporting domain (HHHHHH). Thus, the structure of the fusion protein of the invention is as follows:

(SEQ. No. 41)
TRANS1-LINKER14-UROKIN-MMP-(TRAIL 121-281)

The amino acid sequence and the DNA encoding sequence comprising codons optimized for expression in E. coli are, respectively SEQ. No. 14 and SEQ. No. 70 as shown in the attached Sequence Listing.

The amino acid sequence SEQ. No. 14 of the structure described above was used as a template to generate its coding DNA sequence SEQ. No. 70. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 15

### Fusion Protein of SEQ. No. 15

The protein of SEQ. No. 15 is a fusion protein having the length of 200 amino acids and the mass of 23.3 kDa, wherein domain (a) is the sequence of TRAIL 121-281, and domain (b) of the effector peptide is the 14-amino acids lytic peptide (SEQ. No. 41) is attached at the N-terminus of domain (a).

Additionally, between domain (b) and domain (a) there are sequentially incorporated steric linker (C), 8-arginine transporting sequence (RRRRRRRR), urokinase cleavage site (RVVR) and metalloprotease cleavage site (PLGLAG). Additionally, histidine transporting sequence (HHHHHH) is attached at the N-terminus of the effector peptide. Thus, the structure of the fusion protein of the invention is as follows:

(SEQ. No. 41)
TRANS1-LINKER14-TRANS3-UROKIN-MMP-(TRAIL 121-281)

The amino acid sequence and the DNA encoding sequence comprising codons optimized for expression in E. coli are, respectively SEQ. No. 15 and SEQ. No. 71 as shown in the attached Sequence Listing.

The amino acid sequence SEQ. No. 15 of the structure described above was used as a template to generate its coding DNA sequence SEQ. No. 71. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 16

### Fusion Protein of SEQ. No. 16

The protein of SEQ. No. 16 is a fusion protein having the length of 202 amino acids and the mass of 23.1 kDa, wherein domain (a) is the sequence of TRAIL 121-281, and domain (b) of the effector peptide is the synthetic 14-amino acids lytic peptide (SEQ. No. 41) attached at the C-terminus of domain (a).

Additionally, between domain (a) and domain (b) there are sequentially incorporated steric linker (GGGGSGGGG), metalloprotease cleavage site (PLGLAG), urokinase cleavage site (RVVR) and 8-arginine transporting sequence (RRRRRRRR). Thus, the structure of the fusion protein of the invention is as follows:

(SEQ. No. 41)
(TRAIL 121-281)-LINKER10-MMP-UROKIN TRANS3-

The amino acid sequence and the DNA encoding sequence comprising codons optimized for expression in E. coli are, respectively SEQ. No. 16 and SEQ. No. 72 as shown in the attached Sequence Listing.

The amino acid sequence SEQ. No. 16 of the structure described above was used as a template to generate its coding DNA sequence SEQ. No. 72. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed both with histidine tag (Ex. 16^{a}) and without histidine tag (Ex. 16^{b}).

### Example 17

### Fusion Protein of SEQ. No. 17

The protein of SEQ. No. 17 is a fusion protein having the length of 208 amino acids and the mass of 23.5 kDa, wherein domain (a) is the sequence of TRAIL 116-281, and domain (b) of the effector peptide is the 26-amino acids hybride peptide cecropin A-melittin (SEQ. No. 42) attached at the N-terminus of domain (a).

Additionally, between domain (b) and domain (a) there are sequentially incorporated urokinase cleavage site (RWR) and metalloprotease cleavage site (PLGLAG). Thus, the structure of the fusion protein of the invention is as follows:

(SEQ. No. 42)
-UROKIN-MMP-(TRAIL116-281)

The amino acid sequence and the DNA encoding sequence comprising codons optimized for expression in E. coli are, respectively SEQ. No. 17 and SEQ. No. 73 as shown in the attached Sequence Listing.

The amino acid sequence SEQ. No. 17 of the structure described above was used as a template to generate its coding DNA sequence SEQ. No. 73. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 18

### Fusion Protein of SEQ. No. 18

The protein of SEQ. No. 18 is a fusion protein having the length of 203 amino acids and the mass of 23.6 kDa, wherein domain (a) is the sequence of TRAIL 116-281, and domain (b) of the effector peptide is the 27-amino acids peptide hCAP-18/LL-37 (SEQ. No. 43) attached at the N-terminus of domain (a).

Additionally between domain (b) and domain (a) there are sequentially incorporated urokinase cleavage site (RWR) and metalloprotease cleavage site (PLGLAG). Thus, the structure of the fusion protein of the invention is as follows:

(SEQ. No. 43)
-UROKIN-MMP-(TRAIL116-281)

The amino acid sequence and the DNA encoding sequence comprising codons optimized for expression in E. coli are, respectively SEQ. No. 18 and SEQ. No. 74 as shown in the attached Sequence Listing.

The amino acid sequence SEQ. No. 18 of the structure described above was used as a template to generate its coding DNA sequence SEQ. No. 74. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 19

### Fusion Protein of SEQ. No. 19

The protein of SEQ. No. 19 is a fusion protein having the length of 203 amino acids and the mass of 23.3 kDa, wherein domain (a) is the sequence of TRAIL 116-281, and domain (b) of the effector peptide is the 27-amino acids peptide BAMP-28 (SEQ. No. 44) attached at the N-terminus of domain (a).

Additionally, between domain (b) and domain (a) there are sequentially incorporated urokinase cleavage site (RWR) and metalloprotease cleavage site (PLGLAG). Thus, the structure of the fusion protein of the invention is as follows:

(SEQ. No. 44)
-UROKIN-MMP-(TRAIL116-281)

The amino acid sequence and the DNA encoding sequence comprising codons optimized for expression in E. coli are, respectively SEQ. No. 19 and SEQ. No. 75 as shown in the attached Sequence Listing.

The amino acid sequence SEQ. No. 19 of the structure described above was used as a template to generate its coding DNA sequence SEQ. No. 75. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 20

### Fusion Protein of SEQ. No. 20

The protein of SEQ. No. 20 is a fusion protein having the length of 200 amino acids and the mass of 22.8 kDa, wherein domain (a) is the sequence of TRAIL 121-281, and domain (b) of the effector peptide is the 24-amino acids analogue of isoform C of the lytic peptide from Entamoeba histolytica (SEQ. No. 45) attached at the C-terminus of domain (a).

Additionally, between domain (a) and domain (b) there are sequentially incorporated steric linker (GGSGG), metalloprotease cleavage site (PLGLAG) and urokinase cleavage site (RVVR). Thus, the structure of the fusion protein of the invention is as follows:

(SEQ. No. 45)
(TRAIL121-281)-LINKER6-MMP-UROKIN-

The amino acid sequence and the DNA encoding sequence comprising codons optimized for expression in E. coli are, respectively SEQ. No. 20 and SEQ. No. 76 as shown in the attached Sequence Listing.

The amino acid sequence SEQ. No. 20 of the structure described above was used as a template to generate its coding DNA sequence SEQ. No. 76. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 21

### Fusion Protein of SEQ. No. 20

The protein of SEQ. No. 20 is a fusion protein having the length of 200 amino acids and the mass of 22.8 kDa, wherein domain (a) is the sequence of TRAIL 121-281, and domain (b) of the effector peptide is the 24-amino acids analogue of isoform A of the lytic peptide from Entamoeba histolytica (SEQ. No. 46) attached at the C-terminus of domain (a).

Additionally, between domain (a) and domain (b) there are sequentially incorporated steric linker (GGSGG), metalloprotease cleavage site (PLGLAG) and urokinase cleavage site (RWR). Thus, the structure of the fusion protein of the invention is as follows:

(SEQ. No. 46)
(TRAIL121-281)-LINKER6-MMP-UROKIN-

The amino acid sequence and the DNA encoding sequence comprising codons optimized for expression in E. coli are, respectively SEQ. No. 21 and SEQ. No. 77 as shown in the attached Sequence Listing.

The amino acid sequence SEQ. No. 21 of the structure described above was used as a template to generate its coding DNA sequence SEQ. No. 77. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 22

### Fusion Protein of SEQ. No. 22

The protein of SEQ. No. 22 is a fusion protein having the length of 200 amino acids and the mass of 22.8 kDa, wherein domain (a) is the sequence of TRAIL 121-281, and domain (b) of the effector peptide is the 24-amino acids analogue of isoform B of a lytic peptide from Entamoeba histolytica (SEQ. No. 47) attached at the C-terminus of domain (a).

Additionally, between domain (a) and domain (b) there are sequentially incorporated steric linker (GGSGG), metalloprotease cleavage site (PLGLAG) and urokinase cleavage site (RVVR). Thus, the structure of the fusion protein of the invention is as follows:

(SEQ. No. 47)
(TRAIL121-281)-LINKER6-MMP-UROKIN-

The amino acid sequence and the DNA encoding sequence comprising codons optimized for expression in E. coli are, respectively SEQ. No. 22 and SEQ. No. 78 as shown in the attached Sequence Listing.

The amino acid sequence SEQ. No. 22 of the structure described above was used as a template to generate its coding DNA sequence SEQ. No. 78. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 23

### Fusion Protein of SEQ. No. 23

The protein of SEQ. No. 23 is a fusion protein having the length of 190 amino acids and the mass of 22.1 kDa, wherein domain (a) is the sequence of TRAIL 121-281, and domain (b) of the effector peptide is the 12-amino acids fragment of HA2 domain of influenza virus haemagglutinin (SEQ. No. 48) attached at the N-terminus of domain (a).

Additionally, between domain (b) and domain (a) there are sequentially incorporated 7-arginine transporting sequence (RRRRRRR), urokinase cleavage site (RVVR) and metalloprotease cleavage site (PLGLAG). Thus, the structure of the fusion protein of the invention is as follows:

(SEQ. No. 48)
-TRANS2-UROKIN-MMP-(TRAIL121-281)

The amino acid sequence and the DNA encoding sequence comprising codons optimized for expression in E. coli are, respectively SEQ. No. 23 and SEQ. No. 79 as shown in the attached Sequence Listing.

The amino acid sequence SEQ. No. 23 of the structure described above was used as a template to generate its coding DNA sequence SEQ. No. 79. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above. The protein was expressed both with histidine tag (Ex. 23^{a}) and without histidine tag (Ex. 23^{b}).

### Example 24

### Fusion Protein of SEQ. No. 24

The protein of SEQ. No. 24 is a fusion protein having the length of 429 amino acids and the mass of 48.6 kDa, wherein domain (a) is the sequence of TRAIL 121-281, and domain (b) of the effector peptide is the 247-amino acids fragment of N-terminal domain of alpha-toxin from Clostridium perfringens with phospholipase C activity (SEQ. No. 49) attached at the N-terminus of domain (a).

Additionally, between domain (b) and domain (a) there are sequentially incorporated steric linker (G), steric linker (GGGGGS), pegylation linker (ASGCGPE) and steric linker (GGGGGS). Thus, the structure of the fusion protein of the invention is as follows:

(SEQ. No. 49)
-LINKER15-LINKER5-PEG2-LINKER5-(TRAIL121-281)

The amino acid sequence and the DNA encoding sequence comprising codons optimized for expression in E. coli are, respectively SEQ. No. 24 and SEQ. No. 80 as shown in the attached Sequence Listing.

The amino acid sequence SEQ. No. 24 of the structure described above was used as a template to generate its coding DNA sequence SEQ. No. 80. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 25

### Fusion Protein of SEQ. No. 25

The protein of SEQ. No. 25 is a fusion protein having the length of 658 amino acids and the mass of 73 kDa, wherein domain (a) is the sequence of TRAIL 121-281, and domain (b) of the effector peptide is the 468-amino acids peptide listeriolysin O (SEQ. No. 50) attached at the N-terminus of domain (a).

Additionally, between domain (b) and domain (a) there are sequentially incorporated steric linker (G), steric linker (GGGGSGGGGGS), furin cleavage site (RKKR), pegylation linker (ASGCGPEG) and steric linker sequence (GGGGGS). Thus, the structure of the fusion protein of the invention is as follows:

(SEQ. No. 50)
-LINKER15-LINKER9-FURIN-PEG2-LINKER5-
(TRAIL121-281)

The amino acid sequence and the DNA encoding sequence comprising codons optimized for expression in E. coli are, respectively SEQ. No. 25 and SEQ. No. 81 as shown in the attached Sequence Listing.

The amino acid sequence SEQ. No. 25 of the structure described above was used as a template to generate its coding DNA sequence SEQ. No. 81. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 26

### Fusion Protein of SEQ. No. 26

The protein of SEQ. No. 26 is a fusion protein having the length of 478 amino acids and the mass of 54 kDa, wherein domain (a) is the sequence of TRAIL 121-281, and domain (b) of the effector peptide is the 289-amino acids phospholipase PC-PLC (SEQ. No. 51) attached at the N-terminus of domain (a).

Additionally, between domain (b) and domain (a) there are sequentially incorporated steric linker (GGGSGGGGGS), furin cleavage site (RKKR), pegylation linker (ASGCGPEG) and steric linker (GGGGGS). Thus, the structure of the fusion protein of the invention is as follows:

(SEQ. No. 51)
-LINKER9-FURIN-PEG2-LINKER5-(TRAIL121-281)

The amino acid sequence and the DNA encoding sequence comprising codons optimized for expression in E. coli are, respectively SEQ. No. 26 and SEQ. No. 82 as shown in the attached Sequence Listing.

The amino acid sequence SEQ. No. 26 of the structure described above was used as a template to generate its coding DNA sequence SEQ. No. 82. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 27

### Fusion Protein of SEQ. No. 27

The protein of SEQ. No. 27 is a fusion protein having the length of 361 amino acids and the mass of 40.2 kDa, wherein domain (a) is the sequence of TRAIL 121-281, and domain (b) of the effector peptide is the 179-amino acids equinatoxin EqTx-II (SEQ. No. 52) attached at the N-terminus of domain (a).

Additionally, between domain (b) and domain (a) there are sequentially incorporated steric linker (GGGGS), furin cleavage site (RKKR), pegylation linker (ASGCGPE) and steric linker (GGGGS). Thus, the structure of the fusion protein of the invention is as follows:

(SEQ. No. 52)
-LINKER4-FURIN-PEG1-LINKER4-(TRAIL121-281)

The amino acid sequence and the DNA encoding sequence comprising codons optimized for expression in E. coli are, respectively SEQ. No. 27 and SEQ. No. 83 as shown in the attached Sequence Listing.

The amino acid sequence SEQ. No. 27 of the structure described above was used as a template to generate its coding DNA sequence SEQ. No. 83. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above. The protein was expressed with histidine tag.

### Example 28

### Fusion Protein of SEQ. No. 28

The protein of SEQ. No. 28 is a fusion protein having the length of 227 amino acids and the mass of 25.5 kDa, wherein domain (a) is the sequence of TRAIL 116-281, and domain (b) of the effector peptide is the 46-amino acids peptide viscotoxin A3 (VtA3) (SEQ. No. 53) attached at the N-terminus of domain (a).

Additionally, between domain (b) and domain (a) there are sequentially incorporated metalloprotease cleavage site (PLGLAG), urokinase cleavage site (RVVR) and steric linker (GGSGG). Thus, the structure of the fusion protein of the invention is as follows:

(SEQ. No. 53)
-MMP-UROKIN-LINKER6-(TRAIL116-281)

The amino acid sequence and the DNA encoding sequence comprising codons optimized for expression in E. coli are, respectively SEQ. No. 28 and SEQ. No. 84 as shown in the attached Sequence Listing.

The amino acid sequence SEQ. No. 28 of the structure described above was used as a template to generate its coding DNA sequence SEQ. No. 84. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 29

### Fusion Protein of SEQ. No. 29

The protein of SEQ. No. 29 is a fusion protein having the length of 224 amino acids and the mass of 24.9 kDa, wherein domain (a) is the sequence of TRAIL 121-281, and domain (b) of the effector peptide is the 46-amino acids peptide viscotoxin A3 (VtA3) (SEQ. No. 53) attached at the C-terminus of domain (a).

Additionally, between domain (a) and domain (b) there are sequentially incorporated steric linker (GGGSGGG), metalloprotease cleavage site (PLGLAG), urokinase cleavage site (RVVR). Thus, the structure of the fusion protein of the invention is as follows:

(SEQ. No. 53)
(TRAIL121-281)-LINKER7-MMP-UROKIN-

The amino acid sequence and the DNA encoding sequence comprising codons optimized for expression in E. coli are, respectively SEQ. No. 29 and SEQ. No. 85 as shown in the attached Sequence Listing.

The amino acid sequence SEQ. No. 29 of the structure described above was used as a template to generate its coding DNA sequence SEQ. No. 85. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 30

### Fusion Protein of SEQ. No. 30

The protein of SEQ. No. 30 is a fusion protein having the length of 200 amino acids and the mass of 22.5 kDa, wherein domain (a) is the sequence of TRAIL 121-281, and domain (b) of the effector peptide is the 33-amino acids active fragment of human perforin (SEQ. No. 54) attached at the C-terminus of domain in (a).

Additionally, between domain (a) and domain (b) is incorporated steric linker sequence (GGGGSG). Thus, the structure of the fusion protein of the invention is as follows:

(SEQ. No. 54)
(TRAIL121-281)-LINKER8-

The amino acid sequence and the DNA encoding sequence comprising codons optimized for expression in E. coli are, respectively SEQ. No. 30 and SEQ. No. 86 as shown in the attached Sequence Listing.

The amino acid sequence SEQ. No. 30 of the structure described above was used as a template to generate its coding DNA sequence SEQ. No. 86. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 31

### Fusion Protein of SEQ. No. 31

The protein of SEQ. No. 31 is a fusion protein having the length of 210 amino acids and the mass of 23.5 kDa, wherein domain (a) is the sequence of TRAIL 121-281, and domain (b) of the effector peptide is the 33-amino acids active fragment of human perforin (SEQ. No. 54) attached at the C-terminus of domain (a).

Additionally, between domain (a) and domain (b) there are sequentially incorporated steric linker (GGGGSG), urokinase cleavage site (RVVR), AND metalloprotease cleavage site (PLGLAG). Thus, the structure of the fusion protein of the invention is as follows:

(SEQ. No. 54)
(TRAIL121-281)-LINKER8-UROKIN-MMP-

The amino acid sequence and the DNA encoding sequence comprising codons optimized for expression in E. coli are, respectively SEQ. No. 31 and SEQ. No. 87 as shown in the attached Sequence Listing.

The amino acid sequence SEQ. No. 31 of the structure described above was used as a template to generate its coding DNA sequence SEQ. No. 87. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above. The protein was expressed with histidine tag.

### Example 32

### Fusion Protein of SEQ. No. 32

The protein of SEQ. No. 32 is a fusion protein having the length of 436 amino acids and the mass of 48 kDa, wherein domain (a) is the sequence of TRAIL 116-281, and domain (b) of the effector peptide is the 251-amino acids parasporin-2 from Bacillus thuringensis (SEQ. No. 55) attached at the N-terminus of domain (a).

Additionally, between domain (b) and domain (a) there are sequentially incorporated urokinase cleavage site (RWR), metalloprotease cleavage site (PLGLAG) and steric linker (GSGGGSGGG). Thus, the structure of the fusion protein of the invention is as follows:

(SEQ. No. 55)
-UROKIN-MMP-LINKER11-(TRAIL116-281)

The amino acid sequence and the DNA encoding sequence comprising codons optimized for expression in E. coli are, respectively SEQ. No. 32 and SEQ. No. 88 as shown in the attached Sequence Listing.

The amino acid sequence SEQ. No. 32 of the structure described above was used as a template to generate its coding DNA sequence SEQ. No. 88. A plasmid containing the coding sequence of DNA was generated and overexpression of the fusion protein was carried out in accordance with the general procedures described above. Overexpression was performed according to the general procedure B, using E. coli BL21 (DE3) or Tuner (DE3) strain from Novagen. The protein was separated by electrophoresis in accordance with the general procedure described above. The protein was expressed with histidine tag.

### Example 33

### Fusion Protein of SEQ. No. 33

The protein of SEQ. No. 33 is a fusion protein having the length of 215 amino acids and the mass of 24.3 kDa, wherein domain (a) is the sequence of TRAIL 121-281, and domain (b) of the effector peptide is the 32-amino acids fusion peptide comprising EGF inhibitor and synthetic lytic peptide (SEQ. No. 56), attached at the C-terminus of domain (a).

Additionally, between domain (a) and domain (b) there are sequentially incorporated steric linker (G), steric linker (CAACAAAC), steric linker, (GGG), metalloprotease cleavage site (PLGLAG), and urokinase cleavage site (RVVR). Thus, the structure of the fusion protein of the invention is as follows:

(SEQ. No. 56)
(TRAIL121-281)-LINKER15-LINKER12-LINKER2-MMP-
UROKIN-

The amino acid sequence and the DNA encoding sequence comprising codons optimized for expression in E. coli are, respectively SEQ. No. 33 and SEQ. No. 89 as shown in the attached Sequence Listing.

The amino acid sequence SEQ. No. 33 of the structure described above was used as a template to generate its coding DNA sequence SEQ. No. 89. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed with histidine tag.

### Example 34

### Fusion Protein of SEQ. No. 91

The protein of SEQ. No. 91 is a fusion protein having the length of 223 amino acids and the mass of 25.2 kDa, wherein domain (a) is the sequence of TRAIL 121-281, and domain (b) of the effector peptide is the 39-amino acids fusion peptide comprising PDGFR inhibitor and synthetic lytic peptide (SEQ. No. 125), attached at the N-terminus of domain (a).

Additionally, between domain (b) and domain (a) there are sequentially incorporated urokinase cleavage site (RVVR) and metalloprotease cleavage site (PLGLAG), steric linker (GG), steric linker (CAAACAAC) and steric linker (SGG). Thus, the structure of the fusion protein of the invention is as follows:

(SEQ. No. 125)
-UROKIN-MMP-LINKER1-LINKER13-LINKER16-
(TRAIL121-281)

The amino acid sequence and the DNA encoding sequence comprising codons optimized for expression in E. coli are, respectively SEQ. No. 91 and SEQ. No. 108 as shown in the attached Sequence Listing.

The amino acid sequence SEQ. No. 91 of the structure described above was used as a template to generate its coding DNA sequence SEQ. No. 108. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed without histidine tag.

### Example 35

### Fusion Protein of SEQ. No. 92

The protein of SEQ. No. 92 is a fusion protein having the length of 223 amino acids and the mass of 25.6 kDa, wherein domain (a) is the sequence of TRAIL 95-281, and domain (b) of the effector peptide is the 14-amino acids fusion synthetic lytic peptide (SEQ. No. 41), attached at the N-terminus of domain (a).

Additionally, between domain (b) and domain (a) there are sequentially incorporated polyarginine transporting domain (RRRRRRR), furin cleavage site (RKKR), steric linker (GGG), and steric linker (CAAACAAC). Thus, the structure of the fusion protein of the invention is as follows:

(SEQ. No. 41)
-TRANS2-FURIN-LINKER2-LINKER13-(TRAIL95-281)

The amino acid sequence and the DNA encoding sequence comprising codons optimized for expression in E. coli are, respectively SEQ. No. 92 and SEQ. No. 109 as shown in the attached Sequence Listing.

The amino acid sequence SEQ. No. 92 of the structure described above was used as a template to generate its coding DNA sequence SEQ. No. 109. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

The protein was expressed without histidine tag.

### Example 36

### Fusion Protein of SEQ. No. 93

The protein of SEQ. No. 93 is a fusion protein having the length of 232 amino acids and the mass of 26.7 kDa, wherein domain (a) is the sequence of TRAIL 95-281, and domain (b) of the effector peptide is the 14-amino acids fusion synthetic lytic peptide (SEQ. No. 41), attached at the N-terminus of domain (a).

Additionally, between domain (b) and domain (a) there are sequentially incorporated polyarginine transporting domain (RRRRRRR), furin cleavage site (RKKR), native furin cleavage site (HRQPRGWEQ) steric linker (GGG), and steric linker (CAAACAAC). Thus, the structure of the fusion protein of the invention is as follows:

(SEQ. No. 41)-TRANS2-FURIN-FURN.NAT-LINKER2-
LINKER13-(TRAIL95-281)

The amino acid sequence and the DNA encoding sequence comprising codons optimized for expression in E. coli are, respectively SEQ. No. 93 and SEQ. No. 110 as shown in the attached Sequence Listing.

The amino acid sequence SEQ. No. 93 of the structure described above was used as a template to generate its coding DNA sequence SEQ. No. 110. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed without histidine tag.

### Example 37

### Fusion Protein of SEQ. No. 94

The protein of SEQ. No. 94 is a fusion protein having the length of 207 amino acids and the mass of 23 kDa, wherein domain (a) is the sequence of TRAIL 121-281, and domain (b) of the effector peptide is the 14-amino acids fusion synthetic lytic peptide (SEQ. No. 41), attached at the C-terminus of domain (a).

Additionally, between domain (a) and domain (b) there are sequentially incorporated steric linker (GGGGSGGGG), metalloprotease cleavage site (PLGLAG), urokinase cleavage site (RVVR), transporting domain (YARAAARQARA) and steric linker (GG). Thus, the structure of the fusion protein of the invention is as follows:

(TRAIL121-281)-LINKER10-MMP-UROKIN-TRANS4-
LINKER1-(SEQ. No. 41)

The amino acid sequence and the DNA encoding sequence comprising codons optimized for expression in E. coli are, respectively SEQ. No. 94 and SEQ. No. 111 as shown in the attached Sequence Listing.

The amino acid sequence SEQ. No. 94 of the structure described above was used as a template to generate its coding DNA sequence SEQ. No. 111. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed without histidine tag.

### Example 38

### Fusion Protein of SEQ. No. 95

The protein of SEQ. No. 95 is a fusion protein having the length of 218 amino acids and the mass of 24.4 kDa, wherein domain (a) is the sequence of TRAIL 121-281, and domain (b) of the effector peptide is the 25-amino acids pleurocidine analogue (SEQ. No. 126), attached at the C-terminus of domain (a).

Additionally, between domain (a) and domain (b) there are sequentially incorporated steric linker (GGGGSGGGG), metalloprotease cleavage site (PLGLAG), urokinase cleavage site (RVVR), transporting domain (YARAAARQARA) and steric linker (GG). Thus, the structure of the fusion protein of the invention is as follows:

(TRAIL121-281)-LINKER10-MMP-UROKIN-TRANS4-
LINKER1-(SEQ. No. 126)

The amino acid sequence and the DNA encoding sequence comprising codons optimized for expression in E. coli are, respectively SEQ. No. 95 and SEQ. No. 112 as shown in the attached Sequence Listing.

The amino acid sequence SEQ. No. 95 of the structure described above was used as a template to generate its coding DNA sequence SEQ. No. 112. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed without histidine tag.

### Example 39

### Fusion Protein of SEQ. No. 96

The protein of SEQ. No. 96 is a fusion protein having the length of 219 amino acids and the mass of 24.5 kDa, wherein domain (a) is the sequence of TRAIL 121-281, and domain (b) of the effector peptide is the 26-amino acids pleurocidine analogue (SEQ. No. 127), attached at the C-terminus of domain (a).

Additionally, between domain (a) and domain (b) there are sequentially incorporated steric linker (GGGGSGGGG), metalloprotease cleavage site (PLGLAG), urokinase cleavage site (RVVR), transporting domain (YARAAARQARA) and steric linker (GG). Thus, the structure of the fusion protein of the invention is as follows:

(TRAIL121-281)-LINKER10-MMP-UROKIN-TRANS4-
LINKER1-(SEQ. No. 127)

The amino acid sequence and the DNA encoding sequence comprising codons optimized for expression in E. coli are, respectively SEQ. No. 96 and SEQ. No. 113 as shown in the attached Sequence Listing.

The amino acid sequence SEQ. No. 96 of the structure described above was used as a template to generate its coding DNA sequence SEQ. No. 113. A plasmid containing the coding sequence of DNA was generated and overexpression of the fusion protein was carried out in accordance with the general procedures described above. Overexpression was performed according to the general procedure A, using E. coli (Tuner (DE3) strain from Novagen. The protein was separated by electrophoresis in accordance with the general procedure described above. The protein was expressed without histidine tag.

### Example 40

### Fusion Protein of SEQ. No. 97

The protein of SEQ. No. 97 is a fusion protein having the length of 212 amino acids and the mass of 23.9 kDa, wherein domain (a) is the sequence of TRAIL 121-281, and domain (b) of the effector peptide is the 17-amino acids synthetic lytic peptide (SEQ. No. 128), attached at the C-terminus of domain (a).

Additionally, between domain (a) and domain (b) there are sequentially incorporated steric linker (GGGGSGGGG), metalloprotease cleavage site (PLGLAG), urokinase cleavage site (RVVR), transporting domain (THRPPMWSPVWP) and steric linker (GGG). Thus, the structure of the fusion protein of the invention is as follows:

(TRAIL121-281)-LINKER10-MMP-UROKIN-TRANS5-
LINKER2-(SEQ. No. 127)

The amino acid sequence and the DNA encoding sequence comprising codons optimized for expression in E. coli are, respectively SEQ. No. 97 and SEQ. No. 114 as shown in the attached Sequence Listing.

The amino acid sequence SEQ. No. 97 of the structure described above was used as a template to generate its coding DNA sequence SEQ. No. 114. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed without histidine tag.

### Example 41

### Fusion Protein of SEQ. No. 98

The protein of SEQ. No. 98 is a fusion protein having the length of 207 amino acids and the mass of 23.3 kDa, wherein domain (a) is the sequence of TRAIL 121-281, and domain (b) of the effector peptide is the 14-amino acids synthetic lytic peptide (SEQ. No. 41), attached at the C-terminus of domain (a).

Additionally, between domain (a) and domain (b) there are sequentially incorporated steric linker (GGGGSGGGG), metalloprotease cleavage site (PLGLAG), urokinase cleavage site (RVVR), transporting domain (YGRKKRRQRRR) and steric linker (GG). Thus, the structure of the fusion protein of the invention is as follows:

(TRAIL121-281)-LINKER10-MMP-UROKIN-TRANS6-
LINKER1-(SEQ. No. 41)

The amino acid sequence and the DNA encoding sequence comprising codons optimized for expression in E. coli are, respectively SEQ. No. 98 and SEQ. No. 115 as shown in the attached Sequence Listing.

The amino acid sequence SEQ. No. 98 of the structure described above was used as a template to generate its coding DNA sequence SEQ. No. 115. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed without histidine tag.

### Example 42

### Fusion Protein of SEQ. No. 99

The protein of SEQ. No. 99 is a fusion protein having the length of 207 amino acids and the mass of 24 kDa, wherein domain (a) is the sequence of TRAIL 121-281, and domain (b) of the effector peptide is the 31-amino acids synthetic peptide (SEQ. No. 129), attached at the C-terminus of domain (a).

Additionally, between domain (a) and domain (b) there are sequentially incorporated steric linker (GGGGSGGGG), metalloprotease cleavage site (PLGLAG), and urokinase cleavage site (RVVR). Thus, the structure of the fusion protein of the invention is as follows:

(TRAIL121-281)-LINKER10-MMP-UROKIN-(SEQ. No. 129)

The amino acid sequence and the DNA encoding sequence comprising codons optimized for expression in E. coli are, respectively SEQ. No. 99 and SEQ. No. 116 as shown in the attached Sequence Listing.

The amino acid sequence SEQ. No. 99 of the structure described above was used as a template to generate its coding DNA sequence SEQ. No. 116. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed without histidine tag.

### Example 43

### Fusion Protein of SEQ. No. 100

The protein of SEQ. No. 100 is a fusion protein having the length of 210 amino acids and the mass of 24.1 kDa, wherein domain (a) is the sequence of TRAIL 121-281, and domain (b) of the effector peptide is the 17-amino acids synthetic peptide (SEQ. No. 130), attached at the C-terminus of domain (a).

Additionally, between domain (a) and domain (b) there are sequentially incorporated steric linker (GGGGSGGGG), metalloprotease cleavage site (PLGLAG), and urokinase cleavage site (RVVR), transporting domain (YGRKKRRQRRR) and steric linker (GG). Thus, the structure of the fusion protein of the invention is as follows:

(TRAIL121-281)-LINKER10-MMP-UROKIN-TRANS6-)
LINKER1-(SEQ. No. 130)

The amino acid sequence and the DNA encoding sequence comprising codons optimized for expression in E. coli are, respectively SEQ. No. 100 and SEQ. No. 117 as shown in the attached Sequence Listing.

The amino acid sequence SEQ. No. 100 of the structure described above was used as a template to generate its coding DNA sequence SEQ. No. 117. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed without histidine tag.

### Example 44

### Fusion Protein of SEQ. No. 101

The protein of SEQ. No. 101 is a fusion protein having the length of 211 amino acids and the mass of 23.7 kDa, wherein domain (a) is the sequence of TRAIL 121-281, and domain (b) of the effector peptide is the 29-amino acids synthetic lytic peptide (SEQ. No. 131), attached at the C-terminus of domain (a).

Additionally, between domain (a) and domain (b) there are sequentially incorporated steric linker (GGGGSGGGG), metalloprotease cleavage site (PLGLAG), and urokinase cleavage site (RVVR) and steric linker (GG). Thus, the structure of the fusion protein of the invention is as follows:

(TRAIL121-281)-LINKER10-UROKIN-LINKER1-
(SEQ. No. 131)

The amino acid sequence and the DNA encoding sequence comprising codons optimized for expression in E. coli are, respectively SEQ. No. 101 and SEQ. No. 118 as shown in the attached Sequence Listing.

The amino acid sequence SEQ. No. 101 of the structure described above was used as a template to generate its coding DNA sequence SEQ. No. 118. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed without histidine tag.

### Example 45

### Fusion Protein of SEQ. No. 102

The protein of SEQ. No. 102 is a fusion protein having the length of 234 amino acids and the mass of 26.2 kDa, wherein domain (a) is the sequence of TRAIL 121-281, and two domains (b) of the effector peptide are the 25-amino acids melittin peptide (SEQ. No. 132) and 14-amino acids synthetic lytic peptide (SEQ. No. 41), attached sequentially at the C-terminus of domain (a).

Additionally, between domain (a) and domain (b) there are sequentially incorporated steric linker (GGGGSGGGG), metalloprotease cleavage site (PLGLAG), and urokinase cleavage site (RVVR) and steric linker (GG). Additionally a steric linker (GGGGS) is incorporated between two effector domains.

Thus, the structure of the fusion protein of the invention is as follows:

(TRAIL121-281)-LINKER10-MMP-UROKIN-LINKER1-
(SEQ. No. 132)-LINKER4-(SEQ. No. 41)

The amino acid sequence and the DNA encoding sequence comprising codons optimized for expression in E. coli are, respectively SEQ. No. 102 and SEQ. No. 119 as shown in the attached Sequence Listing.

The amino acid sequence SEQ. No. 102 of the structure described above was used as a template to generate its coding DNA sequence SEQ. No. 119. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed without histidine tag.

### Example 46

### Fusion Protein of SEQ. No. 103

The protein of SEQ. No. 103 is a fusion protein having the length of 205 amino acids and the mass of 23 kDa, wherein domain (a) is the sequence of TRAIL 121-281, and domain (b) of the effector peptide is the 25-amino acids melittin peptide (SEQ. No. 132), attached at the C-terminus of domain (a).

Additionally, between domain (a) and domain (b) there are sequentially incorporated steric linker (GGGGSGGGG), metalloprotease cleavage site (PLGLAG) and urokinase cleavage site (RVVR). Thus, the structure of the fusion protein of the invention is as follows:

(TRAIL121-281)-LINKER10-MMP-UROKIN-(SEQ. No. 132)

The amino acid sequence and the DNA encoding sequence comprising codons optimized for expression in E. coli are, respectively SEQ. No. 103 and SEQ. No. 120 as shown in the attached Sequence Listing.

The amino acid sequence SEQ. No. 103 of the structure described above was used as a template to generate its coding DNA sequence SEQ. No. 120. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed without histidine tag.

### Example 47

### Fusion Protein of SEQ. No. 104

The protein of SEQ. No. 104 is a fusion protein having the length of 215 amino acids and the mass of 24.5 kDa, wherein domain (a) is the sequence of TRAIL 121-281, and domain (b) of the effector peptide is the 25-amino acids melittin peptide (SEQ. No. 132), attached at the C-terminus of domain (a).

Additionally, between domain (a) and domain (b) there are sequentially incorporated steric linker (GGGGSGGGG), metalloprotease cleavage site (PLGLAG), urokinase cleavage site (RVVR), polyarginine transporting domain (RRRRRRRR) and steric linker (GG). Thus, the structure of the fusion protein of the invention is as follows:

(TRAIL121-281)-LINKER10-MMP-UROKIN-TRANS3-LINKER1-
(SEQ. No. 132)

The amino acid sequence and the DNA encoding sequence comprising codons optimized for expression in E. coli are, respectively SEQ. No. 104 and SEQ. No. 121 as shown in the attached Sequence Listing.

The amino acid sequence SEQ. No. 104 of the structure described above was used as a template to generate its coding DNA sequence SEQ. No. 121. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed without histidine tag.

### Example 48

### Fusion Protein of SEQ. No. 105

The protein of SEQ. No. 105 is a fusion protein having the length of 215 amino acids and the mass of 24.4 kDa, wherein domain (a) is the sequence of TRAIL 121-281, and domain (b) of the effector peptide is the 25-amino acids melittin peptide (SEQ. No. 132), attached at the C-terminus of domain (a).

Additionally, between domain (a) and domain (b) there are sequentially incorporated steric linker (GGGGSGGGG), metalloprotease cleavage site (PLGLAG), urokinase cleavage site (RVVR) and steric linker (GG). Additionally, to the C-terminus of domain (b) is attached a polyarginine transporting domain (RRRRRRRR), forming C-terminal fragment of entire construct.

Thus, the structure of the fusion protein of the invention is as follows:

(TRAIL121-281)-LINKER10-MMP-UROKIN-LINKER1-
(SEQ. No. 132)-TRANS3

The amino acid sequence and the DNA encoding sequence comprising codons optimized for expression in E. coli are, respectively SEQ. No. 105 and SEQ. No. 122 as shown in the attached Sequence Listing.

The amino acid sequence SEQ. No. 105 of the structure described above was used as a template to generate its coding DNA sequence SEQ. No. 122. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above.

The protein was expressed without histidine tag.

### Example 49

### Fusion Protein of SEQ. No. 106

The protein of SEQ. No. 106 is a fusion protein having the length of 203 amino acids and the mass of 23.3 kDa, wherein domain (a) is the sequence of TRAIL 121-281, and domain (b) of the effector peptide is the 15-amino acids synthetic lytic peptide (SEQ. No. 35), attached at the C-terminus of domain (a).

Additionally, between domain (a) and domain (b) there are sequentially incorporated steric linker (GGGGSGGGG), metalloprotease cleavage site (PLGLAG), urokinase cleavage site (RWR) and polyarginine transporting domain (RRRRRRRR).

Thus, the structure of the fusion protein of the invention is as follows:

(TRAIL121-281)-LINKER10-MMP-UROKIN-TRANS3-
(SEQ. No. 35)

The amino acid sequence and the DNA encoding sequence comprising codons optimized for expression in E. coli are, respectively SEQ. No. 106 and SEQ. No. 123 as shown in the attached Sequence Listing.

The amino acid sequence SEQ. No. 106 of the structure described above was used as a template to generate its coding DNA sequence SEQ. No. 123. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed without histidine tag.

### Example 50

### Fusion Protein of SEQ. No. 107

The protein of SEQ. No. 107 is a fusion protein having the length of 208 amino acids and the mass of 23.7 kDa, wherein domain (a) is the sequence of TRAIL 121-281, and domain (b) of the effector peptide is the 15-amino acids synthetic lytic peptide (SEQ. No. 35), attached at the C-terminus of domain (a).

Additionally, between domain (a) and domain (b) there are sequentially incorporated steric linker (GGGGSGGGG), metalloprotease cleavage site (PLGLAG), urokinase cleavage site (RVVR), transporting domain (YGRKKRRQRRR) and steric linker (GG).

Thus, the structure of the fusion protein of the invention is as follows:

(TRAIL121-281)-LINKER10-MMP-UROKIN-TRANS6-LINKER1-
(SEQ. No. 35)

The amino acid sequence and the DNA encoding sequence comprising codons optimized for expression in E. coli are, respectively SEQ. No. 107 and SEQ. No. 124 as shown in the attached Sequence Listing.

The amino acid sequence SEQ. No. 107 of the structure described above was used as a template to generate its coding DNA sequence SEQ. No. 124. A plasmid containing the coding sequence of DNA was generated and overexpression of the fusion protein was carried out in accordance with the general procedures described above. Overexpression was performed according to the general procedure A, using E. coli Tuner (DE3) strain from Novagen. The protein was separated by electrophoresis in accordance with the general procedure described above. The protein was expressed without histidine tag.

### Example 51

### Examination of Anti-Tumor Activity of the Fusion Proteins

Examination of anti-tumor activity of the fusion proteins was carried out in vitro in a cytotoxicity assay on tumor cell lines and in vivo in mice. For comparison purposes, rhTRAIL114-281 protein and placebo were used.

### 1. Measurement of Circular Dichroism: Determination of Secondary Structures Composition of the Obtained Proteins

Quality of the preparations of fusion proteins in terms of their structures was determined by circular dichroism for the fusion proteins of Ex. 23, Ex. 11, and Ex. 13.

Circular dichroism is used for determination of secondary structures and conformation of proteins. CD method uses optical activity of the protein structures, manifested in rotating the plane of polarization of light and the appearance of elliptical polarization. CD spectrum of proteins in far ultraviolet (UV) provides precise data on the conformation of the main polypeptide chain.

### Dialysis

Samples of the protein to be analysed, after formulation into a buffer consisting of 50 mM Tris-HCl pH 8.0, 100 mM NaCl, 10% glycerol, 0.1 mM ZnCl₂, 80 mM saccharose, 5 mM DTT, were dialysed in dialysis bags (Sigma-Aldrich) with cut-off 12 kDa. Dialysis was performed against 100 fold excess (v/v) of buffer with respect to protein preparations, with stirring for several hours at 4′C. After dialysis was completed, each preparation was centrifuged (25 000 rpm., 10 min., 4° C.) and supernatants were collected. Protein concentration in the samples thus obtained was determined by Bradford method.

Measurement of circular dichroism for proteins in the concentration range of 0.1-2.7 mg/ml was performed on Jasco J-710 spectropolarimeter, in a quartz cuvette with optical way 0.2 mm or 1 mm. The measurement was performed under the flow of nitrogen at 7 l/min, which allowed to perform the measurement in the wavelength range from 195 to 250 nm. Parameters of the measurement: spectral resolution of −1 nm; half width of the light beam 1 nm; sensitivity 20 mdeg, the averaging time for one wavelength-8 s, scan speed 10 nm/min.

Obtained spectra were analyzed numerically in the range of 193-250 nm using CDPro software. Points for which the voltage at the photomultiplier exceeded 700 V were omitted, due to too low signal to noise ratio in this wavelength range.

The data obtained served for calculations of particular secondary structures content in the analyzed proteins with use of CDPro software (Table 1).

TABLE 1
Content of secondary structures in the analyzed proteins..

| | NRMSD | | | | |
|---|---|---|---|---|---|
| Protein | (Exp-Cal) | α-helix | β-sheet | Schift | Disorder |
| Ex. 23 | 0.149 | 3.7% | 42.0% | 21.1% | 33.2% |
| Ex. 11 | 0.079 | 25.1% | 22.7% | 21.2% | 30.9% |
| Ex. 13 | 0.047 | 15.0% | 32.2% | 20.6% | 32.2% |
| hTRAIL* | | 1.94% | 50.97% | 7.74% | 39.35% |
| hTRAIL | 0.389 | 4.9% | 33.7% | 23.1% | 38.3% |

*value obtained on the basis of crystalline structure 1D4V
**values obtained on the basis of crystalline structures 1IKQ, 1R4Q, 1ABR, 3PX8

The control molecule (rhTRAIL114-281) shows CD spectrum characteristic for the proteins with predominantly type β-sheet structures (sharply outlined ellipticity minimum at the wavelength of 220 nm). This confirms the calculation of secondary structure components, suggesting a marginal number of α-helix elements.

The result obtained is also consistent with the data from the crystal structure of hTRAIL protein, and is characteristic for fusion protein of the invention of Ex. 23, wherein beta elements constitute 42% of their structure. For proteins of Ex. 11 and Ex. 13 higher alpha-helix content was observed (additional minimum of the spectrum at wavelength 208 nm). This is due to the presence in constructs of KLAKLAK motifs which have strong amphipathic character and form alpha-helical-like structures. Unfortunately, due to low stability of proteins from Ex. 23, Ex, 11 and Ex. 13 in the buffer for CD measurements and low concentrations of analyzed preparations their spectra are characterized by a high noise level and with low resolution. Therefore, they may not fully reflect the actual situation, and only suggest the result.

### 2. Tests on Cell Lines In Vitro

### Cell Lines

Cell lines were obtained from ATCC and CLS, and then propagated and deposited in the Adamed's Laboratory of Biology Cell Line Bank. During the experiment, cells were routinely checked for the presence of Mycoplasma by PCR technique using the kit Venor® GeM Mycoplasma PCR Detection Kit (Minerva Biolabs, Berlin, Germany). The cultures were maintained at standard conditions: 37° C., 5% CO₂ (in the case of DMEM—10% CO₂), and 85% relative humidity. Particular cell lines were cultured in appropriate media as recommended by ATCC.

TABLE 2
Adherent cells

| | | | number of |
|---|---|---|---|
| | | | cells per well |
| Cell line | Cancer type | Medium | (thousands) |
| Colo 205 | human colorectal | RPMI + 10% FBS + penicillin + | 5 |
| ATCC | cancer | streptomycin | |
| #CCL-222 | | | |
| HT-29 | human colorectal | McCoy's + 10% FBS + penicillin + | 5 |
| ATCC | cancer | streptomycin | |
| # CCL-2 | | | |
| DU-145 | human prostate | RPMI + 10% FBS + penicillin + | 3 |
| ATCC | cancer | streptomycin | |
| # HTB-81 | | | |
| PC-3 | human prostate | RPMI + 10% FBS + penicillin + | 4 |
| ATCC | cancer | streptomycin | |
| # CRL-1435 | | | |
| MCF-7 | human breast | MEM + 10% FBS + penicillin + | 4.5 |
| ATCC | cancer | streptomycin | |
| #HTB-22 | | | |
| MDA-MB-231 | human breast | DMEM + 10% FBS + penicillin + | 4.5 |
| ATCC | cancer | streptomycin | |
| # HTB-26 | | | |
| MDA-MB-435s | human breast | DMEM + 10% FBS + penicillin + | 4 |
| ATCC# HTB-129 | cancer | streptomycin | |
| UM-UC-3 | human bladder | MEM + 10% FBS + penicillin + | 3.5 |
| ATCC | cancer | streptomycin | |
| # CLR-1749 | | | |
| SW780 | human bladder | DMEM + 10% FBS + penicillin + | 3 |
| ATCC | cancer | streptomycin | |
| #CRL-2169 | | | |
| SW620 | human colorectal | DMEM + 10% FBS + penicillin + | 5 |
| ATCC | cancer | streptomycin | |
| #CCL-227 | | | |
| BxPC-3 | human pancreatic | RPMI + 10% FBS + penicillin + | 4.5 |
| ATCC | cancer | streptomycin | |
| #CRL-1687 | | | |
| SK-V-O3 | human ovarian | McCoy's + 10% FBS + penicillin + | 4 |
| ATCC | cancer | streptomycin | |
| # HTB-77 | | | |
| NIH: OVCAR-3 | human ovarian | RPMI + 20% FBS + 0.01 mg/ml | 7 |
| ATCC | cancer | insulina + penicillin + streptomycin | |
| #HTB-161 | | | |
| HepG2 | human liver | MEM + 10% FBS + penicillin + | 7 |
| ATCC | hepatoma | streptomycin | |
| # HB-8065 | | | |
| 293 | Human embrional | MEM + 10% FBS + penicillin + | 4 |
| ATCC | kidney cells | streptomycin | |
| #CLR-1573 | | | |
| ACHN | human kidney | MEM + 10% FBS + penicillin + | 4 |
| ATCC | cancer | streptomycin | |
| #CCL-222 | | | |
| CAKI 1 | human kidney | McCoy's + 10% FBS + penicillin + | 3.5 |
| ATCC | cancer | streptomycin | |
| #HTB-46 | | | |
| CAKI 2 | human kidney | McCoy's + 10% FBS + penicillin + | 3.5 |
| ATCC | cancer | streptomycin | |
| # HTB 47 | | | |
| NCI-H69AR | human small cell | RPMI + 10% FBS + penicillin + | 10 |
| ATCC | lung cancer | streptomycin | |
| #CRL-11351 | | | |
| HT144 | human melanoma | McCoy's + 10% FBS + penicillin + | 7 |
| ATCC | cells | streptomycin | |
| # HTB-63 | | | |
| NCI-H460 | human lung | RPMI + 10% FBS + penicillin + | 2.5 |
| ATCC | cancer | streptomycin | |
| #HTB-177 | | | |
| A549 | human lung | RPMI + 10% FBS + penicillin + | 2.5 |
| ATCC | cancer | streptomycin | |
| # CCL-185 | | | |
| MES-SA | human uterine | McCoy's + 10% FBS + penicillin + | 3.5 |
| ATCC | sarcoma | streptomycin | |
| # CRL-1976 | | | |
| MES-SA/Dx5 | multidrug- | McCoy's + 10% FBS + penicillin + | 4 |
| ATCC | resistant human | streptomycin | |
| #CRL-1977 | uterine sarcoma | | |
| MES-SA/Mx2 | human uterine | Waymouth's MB 752/1 + McCoy's | 4 |
| ATCC | sarcoma | (1:1) + 10% FBS + penicillin + | |
| #CRL-2274 | | streptomycin | |
| SK-MES-1 ATCC | human lung | MEM + 10% FBS + penicillin + | 5 |
| # HTB-58 | cancer | streptomycin | |
| HCT-116 ATCC | human colorectal | McCoy's + 10% FBS + penicillin + | 3 |
| # CCL-247 | cancer | streptomycin | |
| MCF10A ATCC | mammary | DMEM:F12 + 5% horse plasma + 0.5 μg/ml | 5 |
| # CRL-10317 | epithelial cells | hydrocortisone + 10 μg/ml | |
| | | insuline + 20 ng/ml growth factor | |
| | | EGF | |
| Panc-1 CLS | human pancreatic | DMEM + 10% FBS + penicillin + | 5 |
| 330228 | cancer | streptomycin | |
| Panc03.27 | human pancreatic | RPMI + 10% FBS + penicillin + | 5 |
| ATCC | cancer | streptomycin | |
| # CRL-2549 | | | |
| PLC/PRF/5 CLS | human liver | DMEM + 10% FBS + penicillin + | 5 |
| 330315 | hepatoma | streptomycin | |
| LNCaP | human prostate | RPMI + 10% FBS + penicillin + | 4.5 |
| ATCC | cancer | streptomycin | |
| # CRL-1740 | | | |
| SK-Hep-1 | human liver | RPMI + 10% FBS + penicillin + | 10 |
| CLS300334 | hepatoma | streptomycin | |
| A498 | human kidney | MEM + 10% FBS + penicillin + | 3 |
| CLS 300113 | cancer | streptomycin | |
| HT1080 ATCC | Human | MEM + 10% FBS + penicillin + | 3 |
| #CCL-121 | fibrosarcoma | streptomycin | |
| HUV-EC-C | human umbilical | M199 + 20% FBS + penicillin + 0.05 mg/ml | 8.5 |
| ATCC | vein endothelial | ECGS + 0.1 mg/ml heparine + | |
| # CRL-1730 | cells | penicillin + streptomycin | |

TABLE 3
Nonadherent cells:

| | | | number of |
|---|---|---|---|
| | | | cells per well |
| Cell line | Cancer type | Medium | (thousands) |
| NCI-H69 | human small cell | RPMI + 10% FBS + penicillin + | 22 |
| ATCC # HTB-119 | lung cancer | streptomycin | |
| Jurkat A3 | human leukaemia | RPMI + 10% FBS + penicillin + | 10 |
| ATCC #CRL-2570 | | streptomycin | |
| HL60 | human leukaemia | RPMI + 20% FBS + penicillin + | 10 |
| ATCC # CCL-240 | | streptomycin | |
| CCRF-CEM | human leukaemia | RPMI + 20% FBS + penicillin + | 10 |
| ATCC # CCL-119 | | streptomycin | |

### MTT Cytotoxicity Test

MTT assay is a colorimetric assay used to measure proliferation, viability and cytotoxicity of cells. It consists in decomposition of a yellow tetrazolium salt MTT (4,5-dimethyl-2-thiazolyl)-2,5-diphenyltetrazolium bromide) to the water-insoluble purple dye formazan by mitochondrial enzyme succinate-tetrazolium reductase 1. MTT reduction occurs only in living cells. Data analysis consists in determining IC₅₀ concentration of the protein (in ng/ml), at which the 50% reduction in the number of cells occurs in the population treated compared to control cells. Results were analyzed using GraphPad Prism 5.0 software. The test was performed according to the literature descriptions (Celis, J E, (1998). Cell Biology, a Laboratory Handbook, second edition, Academic Press, San Diego; Yang, Y., Koh, L W, Tsai, J H., (2004); Involvement of viral and chemical factors with oral cancer in Taiwan, Jpn J Clin Oncol, 34 (4), 176-183).

Cell culture medium was diluted to a defined density (10⁴-10⁵ cells per 100 μl). Then 100 μl of appropriately diluted cell suspension was applied to a 96-well plate in triplicates. Thus prepared cells were incubated for 24 h at 37′C in 5% or 10% CO₂, depending on the medium used, and then to the cells (in 100 μl of medium) further 100 μl of the medium containing various concentrations of tested proteins were added. After incubation of the cells with tested proteins over the period of next 72 hours, which is equivalent to 3-4 times of cell division, the medium with the test protein was added with 20 ml of MTT working solution [5 mg/ml], and incubation was continued for 3 h at 37° C. in 5% CO₂. Then the medium with MTT solution was removed, and formazan crystals were dissolved by adding 100 μl of DMSO. After stirring, the absorbance was measured at 570 nm (reference filter 690 nm).

### EZ4U Cytotoxicity Test

EZ4U (Biomedica) test was used for testing cytotoxic activity of the proteins in nonadherent cell lines. The test is a modification of the MTT method, wherein formazan formed in the reduction of tetrazolium salt is water-soluble. Cell viability study was carried out after continuous 72-hour incubation of the cells with protein (seven concentrations of protein, each in triplicates). On this basis IC₅₀ values were determined (as an average of two independent experiments) using the GraphPad Prism 5 software. Control cells were incubated with the solvent only.

The results of in vitro cytotoxicity tests are summarized as IC₅₀ values (ng/ml), which corresponds to the protein concentration at which the cytotoxic effect of fusion proteins is observed at the level of 50% with respect to control cells treated only with solvent. Each experiment represents the average value of at least two independent experiments performed in triplicates. As a criterion of lack of activity of protein preparations the IC₅₀ limit of 2000 ng/ml was adopted. Fusion proteins with an IC₅₀ value above 2000 were considered inactive.

Cells selected for this test included tumor cell lines that are naturally resistant to TRAIL protein (the criterion of natural resistance to TRAIL: IC₅₀ for TRAIL protein >2000), as well as tumor cell lines sensitive to TRAIL protein and resistant to doxorubicin line MES-SA/DX5 as a cancer line resistant to conventional anticancer medicaments.

Undifferentiated HUVEC cell line was used as a healthy control cell line for assessment of the effect/toxicity of the fusion proteins in non-cancer cells.

The results obtained confirm the possibility of overcoming the resistance of the cell lines to TRAIL by administration of certain fusion proteins of the invention to cells naturally resistant to TRAIL. When fusion proteins of the invention were administered to the cells sensitive to TRAIL, in some cases a clear and strong potentiation of the potency of action was observed, which was manifested in reduced IC₅₀ values of the fusion protein compared with IC₅₀ for the TRAIL alone. Furthermore, cytotoxic activity of the fusion protein of the invention in the cells resistant to classical anti-cancer medicament doxorubicin was obtained, and in some cases it was stronger than activity of TRAIL alone.

The IC₅₀ values above 2000 obtained for the non-cancer cell lines show the absence of toxic effects associated with the use of proteins of the invention for healthy cells, which indicates potential low systemic toxicity of the protein.

### Determination of Cytotoxic Activity of Selected Protein Preparations Against Extended Panel of Tumor Cell Lines

Table 4 presents the results of the tests of cytotoxic activity in vitro for selected fusion proteins of the invention against a broad panel of tumor cells from different organs, corresponding to the broad range of most common cancers.

The experimental results are presented as a mean value±standard deviation (SD). All calculations and graphs were prepared using the GraphPad Prism 5.0 software.

Obtained IC₅₀ values confirm high cytotoxic activity of fusion proteins and thus their potential utility in the treatment of cancer.

TABLE 4
Cytotoxic activity of the fusion proteins of the invention

| | Continuous incubation of preparations with cells over 72 h (test MTT, ng/ml) | | | | | | | | | | | |
|---|---|---|---|---|---|---|---|---|---|---|---|---|
| | | | | | | | | | MES-SA/ | | | |
| | | | HCT116 | | MCF10A | | MES-SA | | Dx5 | | SK-MES-1 | |
| Protein | IC₅₀ | ±SD | IC₅₀ | ±SD | IC₅₀ | ±SD | IC₅₀ | ±SD | IC₅₀ | ±SD | IC₅₀ | ±SD |
| | A549 | | | | | | | | | | | |
| rhTRAIL95-281 | >10000 | | 7557 | 3454 | >10000 | | >10000 | | 29.15 | 12.66 | 39.35 | 8.13 |
| Ex. 11^{a} | 115.5 | 60.1 | 6.81 | 4.13 | 103.02 | 18.07 | 7.3 | 1.67 | 1.46 | 0.46 | 1.93 | 0.37 |
| Ex. 13^{a} | 909.35 | 169.21 | 112 | | 750.5 | 156.27 | 110.85 | 9.69 | | | 29.04 | 0.65 |
| Ex. 2^{a} | 170.50 | 7.78 | 45.45 | 14.78 | | | 26.20 | 6.16 | 2.902 | 0.36 | 8.39 | 3.21 |
| Ex. 6^{a} | 915.2 | | 205.8 | | 995.7 | | 126.1 | | | | | |
| Ex. 23^{a} | 1054.7 | 406.3 | 1054.7 | 406.3 | 245.45 | 25.67 | | | 48.06 | 1.75 | 22.1 | 0.18 |
| Ex. 7^{a} | | | 9.465 | | | | | | | | | |
| Ex. 8^{a} | | | 3.894 | | | | 101.3 | | 0.5475 | | 2.058 | |
| Ex. 3^{a} | | | 1878.5 | 171 | | | | | | | | |

| | NCI-H460 | |
|---|---|---|
| rhTRAIL95-281 | 5889 | 111 |
| Ex. 6^{a} | 96.85 | |
| Ex. 2^{a} | 23.50 | 3.54 |

TABLE 4a
Cytotoxic activity of the fusion proteins of the invention

| | Continuous incubation of preparations with cells over 72 h (test MTT, ng/ml) | | | | | | | | | | | |
|---|---|---|---|---|---|---|---|---|---|---|---|---|
| Protein | IC₅₀ | ±SD | IC₅₀ | ±SD | IC₅₀ | ±SD | IC₅₀ | ±SD | IC₅₀ | ±SD | IC₅₀ | ±SD |
| | COLO 205 | | DU 145 | | MDA-MB-231 | | PC 3 | | SW 620 | | SW 780 | |
| rhTRAIL95-281 | | | | | | | | | | | | |
| Ex. 11^{a} | 0.42 | 0.57 | 4.74 | 0.104 | 12.54 | 0.74 | 948 | 42.43 | 735.25 | 45.89 | 0.79 | 0.41 |
| | UM-UC-3 | | HT 29 | | 293 | | ACHN | | CAKI 2 | | BxPC3 | |
| TRAIL 95-281 | 2242 | 1367 | >10000 | | >10000 | | >10000 | | >10000 | | 64.71 | 31.81 |
| Ex. 11^{a} | 0.64 | 0.04 | 4185.5 | 981 | 1152 | 77.78 | 4.86 | 1.06 | 25.42 | 3.22 | 0.43 | 0.114 |
| | HepG2 | | HT 144 | | NCI-H460 | | LNCaP | | OV-CAR-3 | | JURKAT A3 | |
| rhTRAIL 95-281 | >10000 | | 1730 | 218.5 | 5889 | 111 | 2052 | 466 | 963.00 | 144.25 | >10000 | |
| Ex. 11^{a} | 5.63 | 0.45 | 0.26 | 0.065 | 1.8 | 0.34 | 408.15 | 11.8 | 0.114 | 0.07 | 0.29 | 0.24 |

| | PLC/PRF/5 | PANC-1 | | NCI-H460 | |
|---|---|---|---|---|---|
| rhTRAIL95-281 | >9000 | >10000 | | 5889 | 111 |
| Ex. 11^{a} | 436.8 | 142.25 | 56.78 | | |
| Ex. 8^{a} | | | | 5.897 | |

TABLE 4b
Cytotoxic activity of the fusion proteins of the invention

| | Continuous incubation of preparations with cells over 72 h (test MTT, ng/ml) | | | | | | | | | | | |
|---|---|---|---|---|---|---|---|---|---|---|---|---|
| | | | | | | | | | MES- | | | |
| | A549 | | MCF10A | | HCT116 | | MES-SA | | SA/Dx5 | | SK-MES-1 | |
| Protein | IC₅₀ | ±SD | IC₅₀ | ±SD | IC₅₀ | ±SD | IC₅₀ | ±SD | IC₅₀ | ±SD | IC₅₀ | ±SD |
| TRAIL 95-281 | >2000 | | >2000 | | >2000 | | >2000 | | 27.59 | 13.34 | 100.7 | 26.4 |
| Ex. 6^{b} | 915 | | 996 | | 206 | | 126 | | 56.2 | | 53.3 | |
| Ex. 23^{b} | 550 | | 1342 | | 245 | 26 | 99 | | 48.1 | 1.8 | 22.11 | 0.18 |
| Ex. 16^{b} | 10.96 | 2.14 | 4.71 | 1.26 | 1.5 | 0.19 | 0.08 | 0.07 | 0.0001 | | 0.06 | 0.03 |
| Ex. 2^{b} | 171 | 8 | | | 45.5 | 14.8 | 26.2 | 6.2 | 2.9 | 0.36 | 8.39 | 3.21 |
| Ex. 7^{b} | | | >2000 | | 9.47 | | | | 3.48 | | 7.2 | |
| Ex. 8^{b} | | | >2000 | | 3.89 | | 101 | | 0.55 | | 2.06 | |
| Ex. 11^{b} | 89.2 | 11.1 | | | 13.73 | 1.34 | | | | | | |
| Ex. 13^{b} | 405 | | | | | | | | | | | |

TABLE 4c
Cytotoxic activity of the fusion proteins of the invention

| | Continuous incubation of preparations with cells over 72 h (test MTT, ng/ml) | | | | | | | | | | | |
|---|---|---|---|---|---|---|---|---|---|---|---|---|
| Protein | IC₅₀ | ±SD | IC₅₀ | ±SD | IC₅₀ | ±SD | IC₅₀ | ±SD | IC₅₀ | ±SD | IC₅₀ | ±SD |

| | SW620 | | Panc-1 | | PLC/PRF/5 | | HT-29 | | Caki-1 | | SK-HEP-1 | |
|---|---|---|---|---|---|---|---|---|---|---|---|---|
| TRAIL 95-281 | >2000 | | >2000 | | >2000 | | >2000 | | 13.42 | 2.16 | >2000 | |
| Ex. 11^{b} | 325 | 24 | 10.87 | 1.8 | 46.4 | 20 | 893 | | 11.57 | | 75.1 | 11.3 |
| Ex. 16^{b} | 1688 | 917 | 0.68 | 0.93 | 2.89 | 2.02 | 1063 | 480 | 3.29 | 1.44 | 4.27 | 2.36 |
| Ex. 13^{b} | 4.42 | | 26 | | 5.8 | | | | | | | |
| | Caki-2 | | SK-OV-3 | | BxPC-3 | | HT-144 | | OV-CAR-3 | | HT-1080 | |
| TRAIL 95-281 | >2000 | | >2000 | | 60.6 | 22.8 | 1134 | 375 | 963 | 144 | >2000 | |
| Ex. 16^{b} | 3.54 | 0.52 | 161.2 | 1.8 | 0.55 | 0.12 | 0.13 | 0.05 | 0.12 | | 1025 | 395 |
| | MES-SA/Mx2 | | Colo205 | | MCF-7 | | MDA-MB-231 | | MDA-MB-435S | | ACHN | |
| TRAIL 95-281 | 38.95 | 6.14 | 59.02 | 21.16 | >2000 | | >2000 | | >2000 | | >2000 | |
| Ex. 16^{b} | 0.0001 | | 0.48 | 0.65 | 1.74 | 0.51 | 1.71 | 1.19 | 0.86 | 1.08 | 0.38 | 0.32 |

TABLE 4d
Cytotoxic activity of the fusion proteins of the invention

| | Continuous incubation of preparations with cells over 72 h (test MTT, ng/ml) | | | | | | | | | | | |
|---|---|---|---|---|---|---|---|---|---|---|---|---|
| | NCI-H460 | | HepG2 | | Panc03.27 | | A498 | | HUV-EC-C | | | |
| Protein | IC₅₀ | ±SD | IC₅₀ | ±SD | IC₅₀ | ±SD | IC₅₀ | ±SD | IC₅₀ | ±SD | IC₅₀ | ±SD |
| TRAIL 95-281 | 438 | 77 | >2000 | | 315 | | 1611 | 103 | >2000 | | | |
| Ex. 16^{b} | 0.47 | 0.22 | 11.27 | 1.3 | 11.3 | 0.4 | 0.06 | 0.07 | >2000 | | | |
| Ex. 13^{b} | 6.78 | | 13 | | | | | | | | | |
| Ex. 11^{b} | | | | | 42.1 | 17.4 | 2.6 | 0.15 | | | | |
| Ex. 7^{b} | 15.8 | | | | | | | | | | | |
| Ex. 8^{b} | 5.9 | | | | | | | | | | | |

### 3. Antitumor Effectiveness of Fusion Proteins In Vivo on Xenografts

Antitumor activity of protein preparations was tested in a mouse model of human colon cancer Colo 205 and HCT-116, human lung cancer A549 and NCI-H460-Luc, human prostate cancer PC-3, human pancreas cancer Panc-1 and MIA PaCa-2, human liver cancer PCL/PRF/5 and HepG2, and human multidrug resistant MES uterine sarcoma—SA.Dx5.

### Cells

The cells of human lung cancer A549 and NCI-H460-Luc2 and human prostate cancer PC3 were maintained in RPMI1640 medium (HyClone, Logan, Utah, USA) supplemented with 10% fetal calf serum and 2 mM glutamine.

The cells of human colon cancer Colo 205 were maintained in RPMI1640 medium (HyClone, Logan, Utah, USA) (optionally mixed in the ratio of 1:1 with Opto-MEM (Invitrogen, Cat. No. 22600-134) supplemented with 10% fetal calf serum and 2 mM glutamine.

The cells of human pancreas cancer PANC-1, human liver cancer PLC/PRF/5, pancreas cancer MIA PaCa-2 and human colon cancer SW-620 were maintained in DMEM medium (HyClone, Logan, Utah, USA) supplemented with 10% fetal calf serum and 2 mM glutamine.

The cells of human colon cancer HCT-116 were maintained in McCoy's medium (HyClone, Logan, Utah, USA) supplemented with 10% fetal calf serum and 2 mM glutamine.

The cells of multidrug resistant human uterine sarcoma MES-SA.Dx5 were maintained in McCoy's medium (HyClone, Logan, Utah, USA) supplemented with 10% fetal calf serum and 2 mM glutamine, and 1 μM doxorubicin hydrochloride (Sigma, Cat. No. D1515-10MG). Three days before the cells implantation, the cells were cultured in medium without doxorubicin.

The cells of human liver cancer HepG2 were maintained in MEM medium (HyClone, Logan, Utah, USA) supplemented with 10% fetal calf serum and 2 mM glutamine. On the day of mice grafting, the cells were detached from the so support by washing the cells with trypsin (Invitrogen), then the cells were centrifuged at 1300 rpm, 4′C, 8 min., suspended in HBSS buffer (Hanks medium).

On the day of mice grafting, the cells were detached from the support by washing the cells with trypsin (Invitrogen), then the cells were centrifuged at 1300 rpm, 4° C., 8 min., suspended in HBSS buffer (Hanks medium).

### Mice

Examination of antitumor activity of proteins of the invention was conducted on 4-6 week-old (lung cancer model) Cby.Cg-foxn1(nu)/J) mice or 9-10 week old (prostate cancer model) obtained from Centrum Medycyny Doświadczalnej in Bialystok, 4-5 week-old female Crl:SHO-Prkdc^{scid}Hr^{h} mice obtained from Charles River Germany. Mice were kept under specific pathogen-free conditions with free access to food and demineralised water (ad libitum). All experiments on animals were carried in accordance with the guidelines: “Interdisciplinary Principles and Guidelines for the Use of Animals in Research, Marketing and Education” issued by the New York Academy of Sciences' Ad Hoc Committee on Animal Research and were approved by the IV Local Ethics Committee on Animal Experimentation in Warsaw (No. 71/2009).

### the Course and Evaluation of the Experiments

Tumor size was measured using electronic calliper, tumor volume was calculated using the formula: (a²×b)/2, where a=shorter diagonal of the tumor (mm) and b=longer diagonal of the tumor (mm). Inhibition of tumor growth was calculated using the formula:

TGI[%](Tumor growth inhibition)=(WT/WC)×100−100%

wherein WT is the average tumor volume in the treatment group, and WC is the average tumor volume in the control group.

The experimental results are presented as a mean value±standard deviation (SD). All calculations and graphs were prepared using the program GraphPad Prism 5.0.

### Lung Cancer Model C

### Experiment A.

On day 0 Cby.Cg-foxn1(nu)/J mice were grafted subcutaneously (s.c.) in the right side with 5×10⁶ of A549 cells suspended in 0.1 ml of the mixture HBSS:Matrigel 4:1 using syringe with a needle 0.5×25 mm (Bogmark). On the 19 day of the experiment mice were randomized to obtain the average size of tumors in the group of ˜75 mm3 and assigned to treatment groups. The treatment groups were administered with the preparations of fusion protein of the invention of Ex. 11^{a} (15 mg/kg), and rhTRAIL114-281 (20 mg/kg) as a comparison and water for injections as a control. The preparations were administered intravenously (i.v) 6 times once daily every second day. On the 35 day of the experiment mice were sacrificed by disruption of the spinal cord.

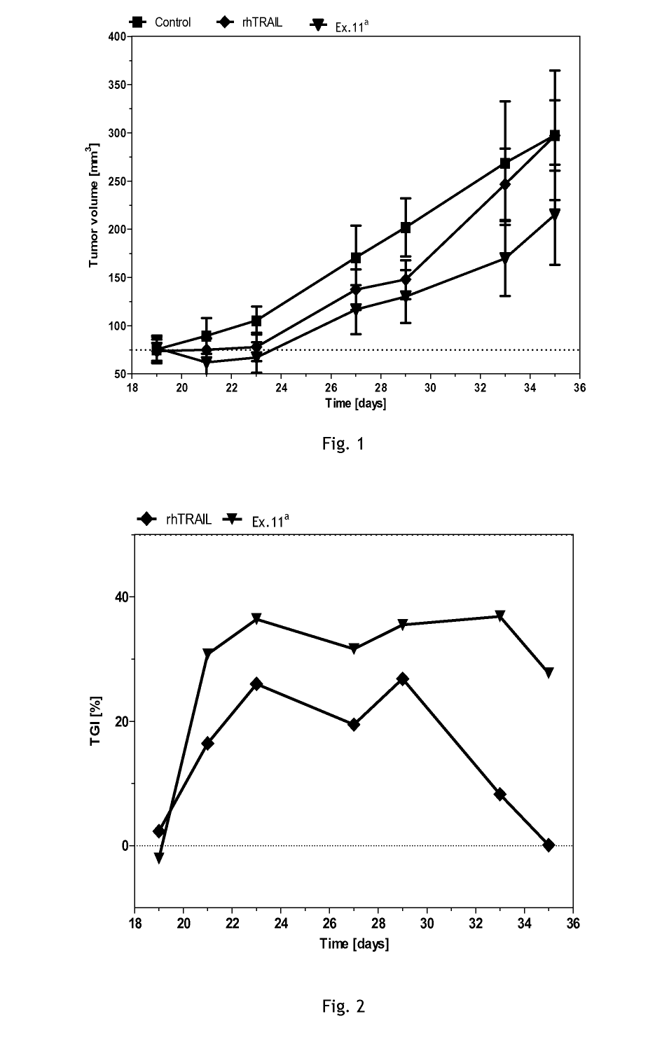

The experimental results are shown on FIG. 1 and FIG. 2 as a diagram of changes of the tumor volume and tumor growth inhibition (% TGI) as the percentage of control in mice treated with fusion protein of the invention of Ex. 11^{a} and comparatively with rhTRAIL114-281.

The experimental results presented in FIGS. 1 and 2 show that administration of the fusion protein of the invention of Ex. 11^{a} caused A549 tumor growth inhibition, with TGI 28% relative to the control on 35 day of experiment. For rhTRAIL114-281 used as the comparative reference, a slight inhibitory effect on tumor cell growth was obtained relative to the control, with TGI at the level of 0%. Thus, fusion proteins of the invention exerted much stronger effect compared to TRAIL alone.

Experiment B. On day 0 Crl:SHO-Prkdc^{scid}Hr^{h} mice were grafted subcutaneously (s.c.) in the right side with 7×10⁶ of A549 cells suspended in 0.1 ml of the mixture HBSS:Matrigel 3:1 using syringe with a needle 0.5×25 mm (Bogmark). On the 17 day of the experiment mice were randomized to obtain the average size of tumors in the group of ˜100-120 mm³ and assigned to treatment groups. The treatment groups were administered with the preparations of fusion protein of the invention of Ex. 11^{a} (40 mg/kg), and rhTRAIL114-281 (20 mg/kg) as a comparison against formulation buffer (19 mM NaH₂PO₄, 81 mM Na₂HPO₄, 50 mM NaCl, 5 mM glutathione, 0.1 mM ZnCl₂, 10% glycerol, pH 7.4) as a control. The preparations were administered intravenously (i.v) 6 times once daily every second day. On the 34 day of the experiment mice were sacrificed by disruption of the spinal cord.

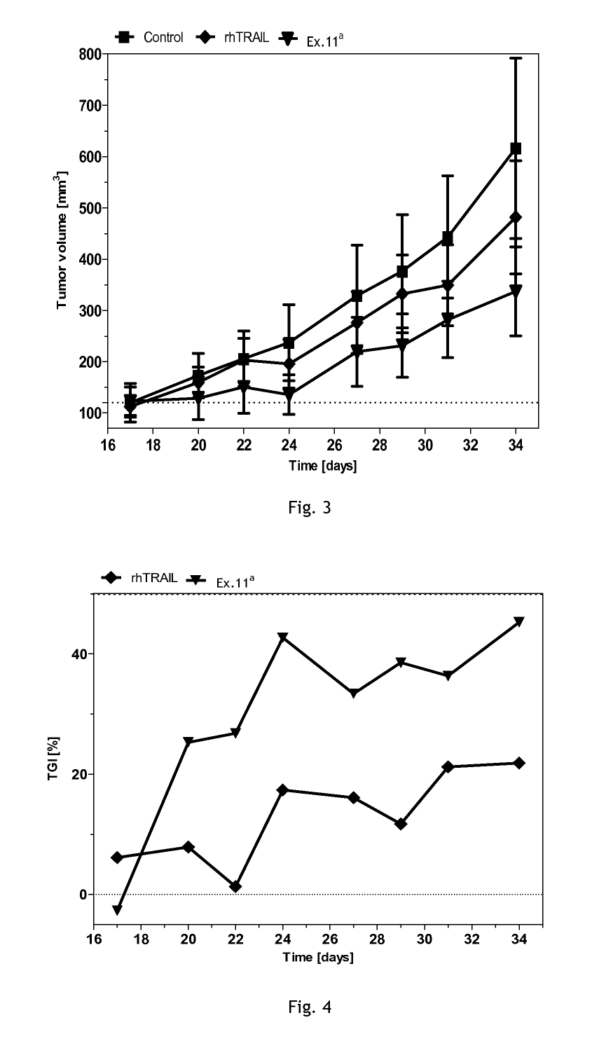

The experimental results are shown on FIG. 3 and FIG. 4 as a diagram of changes of the tumor volume and tumor growth inhibition (% TGI) as the percentage of control in mice treated with fusion protein of the invention of Ex. 11^{d} and comparatively rhTRAIL114-281.

The experimental results presented in FIGS. 3 and 4 show that administration of the fusion protein of the invention of Ex. 11^{a} caused A549 tumor growth inhibition, with TGI 45% relative to the control on 34 day of experiment. For rhTRAIL114-281 used as the comparative reference, a slight inhibitory effect on tumor cell growth was obtained relative to the control, with TGI at the level of 21.8%. Thus, fusion protein of the invention exerted much stronger effect compared to TRAIL alone.

### Experiment C.

On day 0 Crl:SHO-Prkdc^{scid}Hr^{hr} mice were grafted subcutaneously (s.c.) in the right side with 5×10⁶ of NCI-H460-Luc2 cells suspended in 0.1 ml of HBSS buffer using syringe with a needle 0.5×25 mm (Bogmark). On the 11 day of the experiment mice were randomized to obtain the average size of tumors in the group of ˜100-120 mm³ and assigned to treatment groups. The treatment groups were administered with the preparations of fusion protein of the invention of Ex. 11^{a} (40 mg/kg and 30 mg/kg), and rhTRAIL114-281 (20 mg/kg) as a comparison against formulation buffer (19 mM NaH₂PO₄, 81 mM Na₂HPO₄, 50 mM NaCl, 5 mM glutathione, 0.1 mM ZnCl₂, 10% glycerol, pH 7.4) as a control. The preparations were administered intravenously (i.v) 6 times once daily every second day (in case of fusion protein of the invention of Ex. 11^{a} first administration at a dose 40 mg/kg and subsequent at 30 mg/kg. On the 29 day of the experiment mice were sacrificed by disruption of the spinal cord.

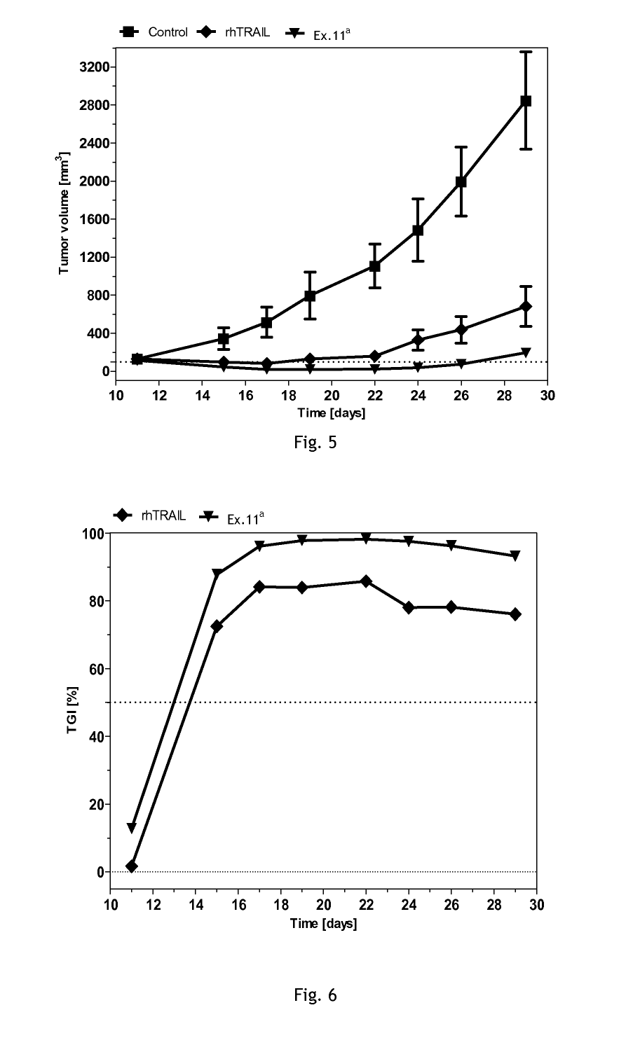

The experimental results are shown on FIG. 5 and FIG. 6 as a diagram of changes of the tumor volume and tumor growth inhibition (% TGI) as the percentage of control in mice treated with fusion protein of the invention of Ex. 11^{a} and comparatively with rhTRAIL114-281.

The experimental results presented in FIGS. 5 and 6 show that administration of the fusion protein of the invention of Ex. 11^{a} caused tumor NCI-H460-Luc2 growth inhibition, with TGI 93% relative to the control on 29 day of experiment. For rhTRAIL114-281 used as the comparative reference, an inhibitory effect on tumor cell growth was obtained relative to the control, with TGI at the level of 76%. Thus, fusion protein of the invention exerted much stronger effect compared to TRAIL alone.

The tested fusion protein did not cause significant side effects manifested by a decrease in body weight of mice (i.e. less than 10% of the baseline body weight). This shows low systemic toxicity of the protein of the invention.

### Prostate Cancer Model

On day 0 Cby.Cg-foxn1(nu)/J mice were grafted subcutaneously (s.c.) in the right side with 5×10⁶ of PC3 cells suspended in 0.18 ml of HBSS buffer and 0.02 ml of Matrigel using syringe with a needle 0.5×25 mm (Bogmark). On the 29 day of the experiment mice were randomized to obtain the average size of tumors in the group of ˜90 mm³ and assigned to treatment groups. The treatment groups were administered with the preparations of fusion protein of the invention of Ex. 11^{a} (15 mg/kg) and 0.9% NaCl as a control. The preparations were administered intravenously (i.v) once daily for 6 days. On the 60 day of the experiment mice were sacrificed by disruption of the spinal cord.

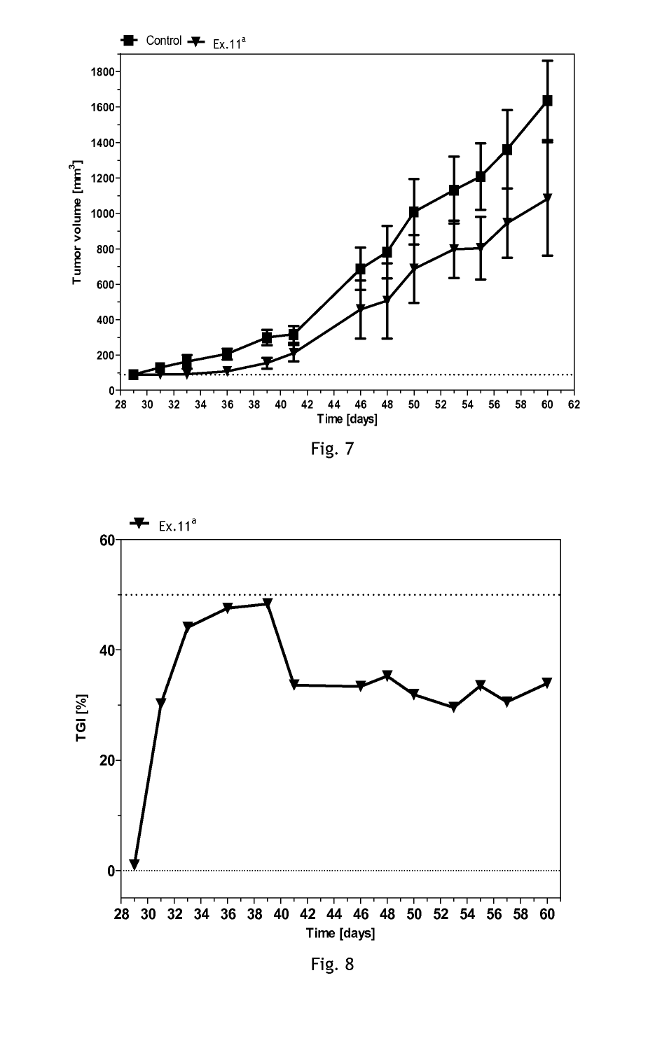

The experimental results are shown on FIG. 7 and FIG. 8 as a diagram of changes of the tumor volume and tumor growth inhibition (% TGI) as the percentage of control in mice treated with fusion protein of the invention of Ex. 11^{a}.

The experimental results presented in FIGS. 7 and 8 show that administration of the fusion protein of the invention of Ex. 11^{a} caused PC3 tumor growth inhibition, with TGI 33% relative to the control on the 60 day of experiment.

The tested fusion protein did not cause significant side effects manifested by a decrease in body weight of mice (i.e. less than 10% of the baseline body weight). This shows low systemic toxicity of the protein of the invention.

### Pancreas Cancer Model

### Experiment A on PANC-1 Cells

On day 0 Crl:SHO-Prkdc^{scid}Hr^{hr} mice were grafted subcutaneously (s.c.) in the right side with 5×10⁶ of PANC-1 cells suspended in 0.1 ml of the mixture HBSS:Matrigel 3:1 using syringe with a needle 0.5×25 mm (Bogmark). On the 31 day of the experiment mice were randomized to obtain the average size of tumors in the group of ˜110 mm³ and assigned to treatment groups. The treatment groups were administered with the preparations of fusion protein of the invention of Ex. 11^{a} (40 mg/kg), and rhTRAIL114-281 (20 mg/kg) as a comparison against formulation buffer (19 mM NaH₂PO₄, 81 mM Na₂HPO₄, 50 mM NaCl, 5 mM glutathione, 0.1 mM ZnCl₂, 10% glycerol, pH 7.4) as a control. The preparations were administered intravenously (i.v) 6 times once daily every second day. On the 42 day of the experiment mice were sacrificed by disruption of the spinal cord.

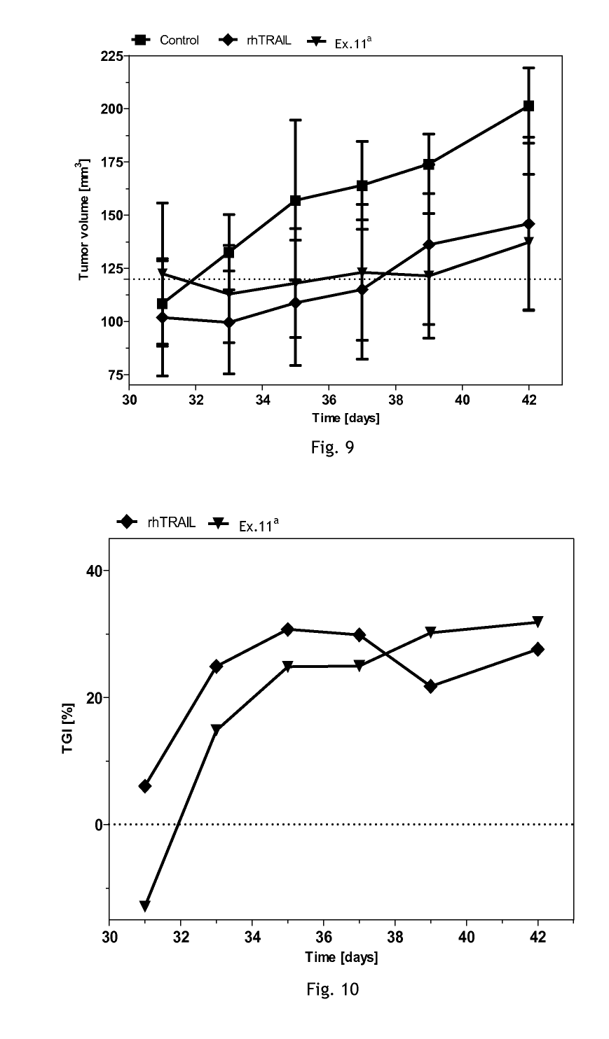

The experimental results are shown on FIG. 9 and FIG. 10 as a diagram of changes of the tumor volume and tumor growth inhibition (% TGI) as the percentage of control in mice treated with fusion protein of the invention of Ex. 11^{a} and comparatively with rhTRAIL114-281.

The experimental results presented in FIGS. 9 and 10 show that administration of the fusion protein of the invention of Ex. 11^{a} caused PANC-1 tumor growth inhibition, with TGI 32.6% relative to the control on 42 day of experiment. For rhTRAIL114-281 used as the comparative reference, a slight inhibitory effect on tumor cell growth was obtained relative to the control, with TGI at the level of 26%. Thus, fusion protein of the invention exerted much stronger effect compared to TRAIL alone.

The tested fusion protein did not cause significant side effects manifested by a decrease in body weight of mice (i.e. less than 10% of the baseline body weight). This shows low systemic toxicity of the protein of the invention.

### Experiment B on MIA PaCa-2 Cells

On day 0 Crl:SHO-Prkdc^{scid}Hr^{hr} mice were grafted subcutaneously (s.c.) in the right flank region with 5×10⁶ of MIA PaCa-2 cells suspended in 0.1 ml of 3:1 mixture HBSS buffer:Matrigel using syringe with a 0.5×25 mm needle (Bogmark). When tumors reached the size of 60-398 mm³ (day 20), mice were randomized to obtain the average size of tumors in the group of ˜170 mm³ and assigned to treatment groups. The treatment groups were administered with the preparations of fusion protein of the invention of Ex. 16^{b} (50 mg/kg) and rhTRAIL114-281 (50 mg/kg) as a comparison and reference compound gemcytabine (Gemzar, Eli Lilly) (50 mg/kg). The preparations were administered intravenously (i.v.) six times every second day, gemcytabine was applied intraperitoneally (i.p.) in the same schedule. The control group received formulation buffer.

When a therapeutic group reached the average tumor size of ˜1000 mm³, mice were sacrificed by the cervical dislocation.

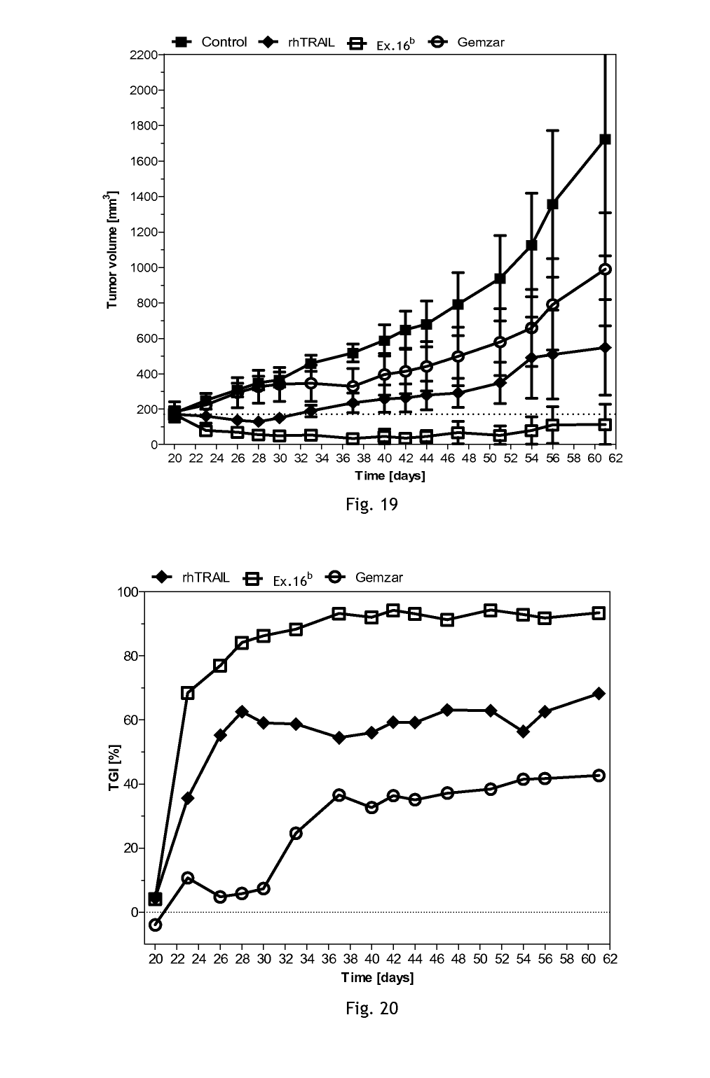

The experimental results obtained in mice Crl:SHO-Prkdc^{scid}Hr^{hr} burdened with MIA PaCa-2 pancreatic carcinoma treated with fusion protein of the invention of Ex. 16^{b} and comparatively with rhTRAIL114-281 and gemcytabine are shown in FIG. 19 as a diagram of changes of the tumor volume and in FIG. 20 which shows tumor growth inhibition (% TGI) as a percentage of control.

The results of the experiment presented in FIGS. 19 and 20 show that administration of the fusion protein of the invention of Ex. 16^{b} caused MIA PaCa-2 tumor growth inhibition, with TGI 93% relative to the control on 61^{th} day of the experiment. For rhTRAIL114-281 and gemcytabine as comparative references, a slight inhibitory effect on tumor cell growth was obtained relative to the control, with TGI at the level of 68% and 42.6%, respectively. Thus, fusion proteins of the invention exerted much stronger effect compared to TRAIL alone and standard chemotherapy.

### Colon Cancer Model

### Experiment A on HCT116 Cells

On day 0 Crl:SHO-Prkdc^{scid}Hr^{hr} mice were grafted subcutaneously (s.c.) in the right side with 5×10⁶ of HCT116 cells suspended in 0.1 ml of HBSS buffer using syringe with a needle 0.5×25 mm (Bogmark). On the 18 day of the experiment mice were randomized to obtain the average size of tumors in the group of −400 mm³ and assigned to treatment groups. The treatment groups were administered with the preparations of fusion protein of the invention of Ex. 11^{a} (35 mg/kg), and rhTRAIL114-281 (20 mg/kg) as a comparison against formulation buffer (5 mM NaH₂PO₄, 95 mM Na₂HPO₄, 200 mM NaCl, 5 mM glutathione, 0.1 mM ZnCl₂, 10% glycerol, 80 mM saccharose, pH 8.0) as a control. The preparations were administered intravenously (i.v) 6 times once daily every second day. On the 32 day of the experiment mice were sacrificed by disruption of the spinal cord.

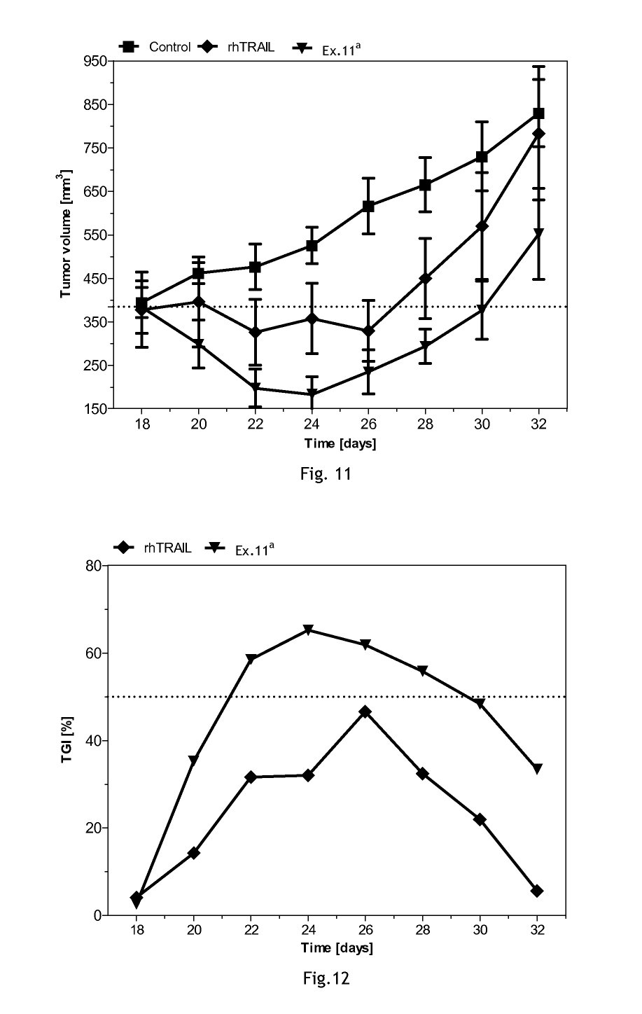

The experimental results are shown on FIG. 11 and FIG. 12 as a diagram of changes of the tumor volume and tumor growth inhibition (% TGI) as a percentage of control in mice treated with fusion protein of the invention of Ex. 11^{a} and comparatively with rhTRAIL114-281.

The experimental results presented in FIGS. 11 and 12 show that administration of the fusion protein of the invention of Ex. 11^{a} caused tumor HCT116 growth inhibition, with TGI 33.5% relative to the control on 32 day of experiment. For rhTRAIL114-281 used as the comparative reference, a slight inhibitory effect on tumor cell growth was obtained relative to the control, with TGI at the level of 5.6%. Thus, fusion protein of the invention exerted much stronger effect compared to TRAIL alone.

The tested fusion protein did not cause significant side effects manifested by a decrease in body weight of mice (i.e. less than 10% of the baseline body weight). This shows low systemic toxicity of the protein of the invention.

### Experiment A1 on HCT116 Cells

On day 0 Crl:SHO-Prkdc^{scid}Hr^{hr} mice were grafted subcutaneously (s.c.) in the right flank region with 5×10⁶ of HCT116 cells suspended in 0.1 ml of the 3:1 mixture HBSS buffer:Matrigel using syringe with a 0.5×25 mm needle (Bogmark). When tumors reached the size of 71-432 mm³ (day 13), mice were randomized to obtain the average size of tumors in the group of ˜180 mm³ and assigned to treatment groups. The treatment groups were administered with the preparations of fusion protein of the invention of Ex. 16^{b} (90 mg/kg) and rhTRAIL114-281 (65 mg/kg) as a comparison. The preparations were administered intravenously (i.v.) six times every second day. The control group received formulation buffer.

When an experimental group reached the average tumor size of −1000 mm³, mice were sacrificed by cervical dislocation.

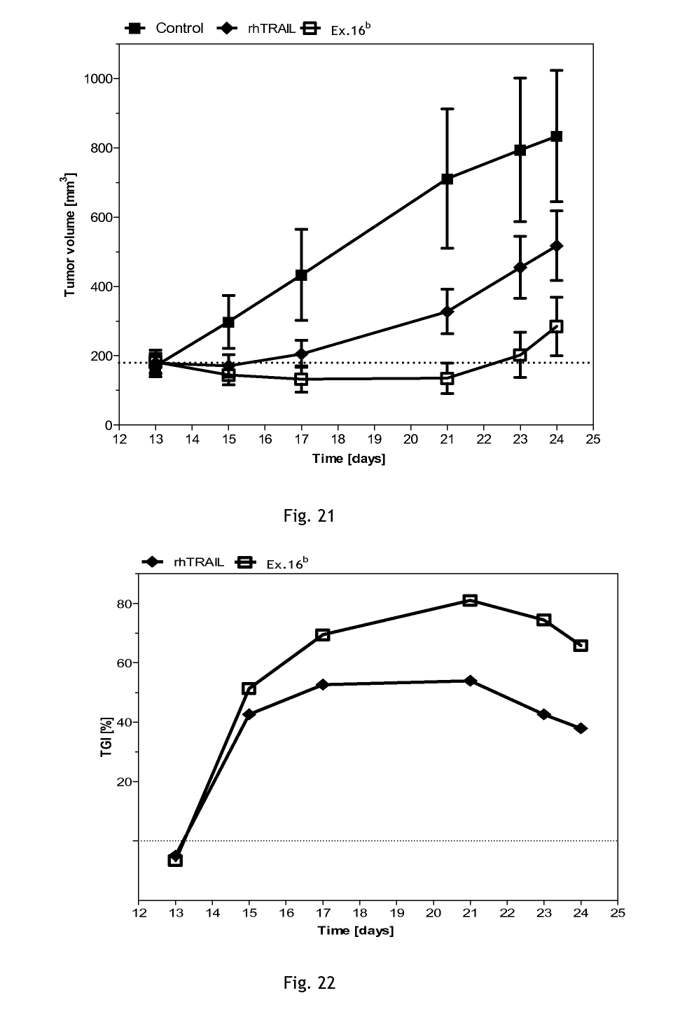

The experimental results obtained in mice Crl:SHO-Prkdc^{scid}Hr^{hr} burdened with HCT116 colon cancer treated with fusion proteins of the invention of Ex. 16^{b} and comparatively with rhTRAIL114-281 are shown in FIG. 21 as a diagram of changes of the tumor volume and in FIG. 22 which shows tumor growth inhibition (% TGI) as a percentage of control.

The results of experiments presented in FIGS. 21 and 22 show that administration of the fusion protein of the invention of Ex. 16^{b} caused HCT116 tumor growth inhibition, with TGI 65.8% relative to the control on 24^{th} day of the experiment. For rhTRAIL114-281 used as the comparative reference, a slight inhibitory effect on tumor cell growth was obtained relative to the control, with TGI at the level of 37.9%. Thus, fusion proteins of the invention exerted much stronger effect compared to TRAIL alone.

### Experiment B on SW620 Cells

On day 0 Crl:SHO-Prkdc^{scid}Hr^{hr} mice were grafted subcutaneously (s.c.) in the right side with 5×10⁶ of SW620 cells suspended in 0.1 ml of the mixture HBSS:Matrigel 3:1 using syringe with a needle 0.5×25 mm (Bogmark). On the 17 day of the experiment mice were randomized to obtain the average size of tumors in the group of ˜320 mm³ and assigned to treatment groups. The treatment groups were administered with the preparation of fusion protein of the invention of Ex. 16^{a} (20 mg/kg) and rhTRAIL114-281 (30 mg/kg) as a comparison against formulation buffer (19 mM NaH₂PO₄, 81 mM Na₂HPO₄, 50 mM NaCl, 5 mM glutathione, 0.1 mM ZnCl₂, 10% glycerol, pH 7.4) as a control. The preparations were administered intravenously (i.v) 6 times once daily every second day. On the 31 day of the experiment mice were sacrificed by disruption of the spinal cord.

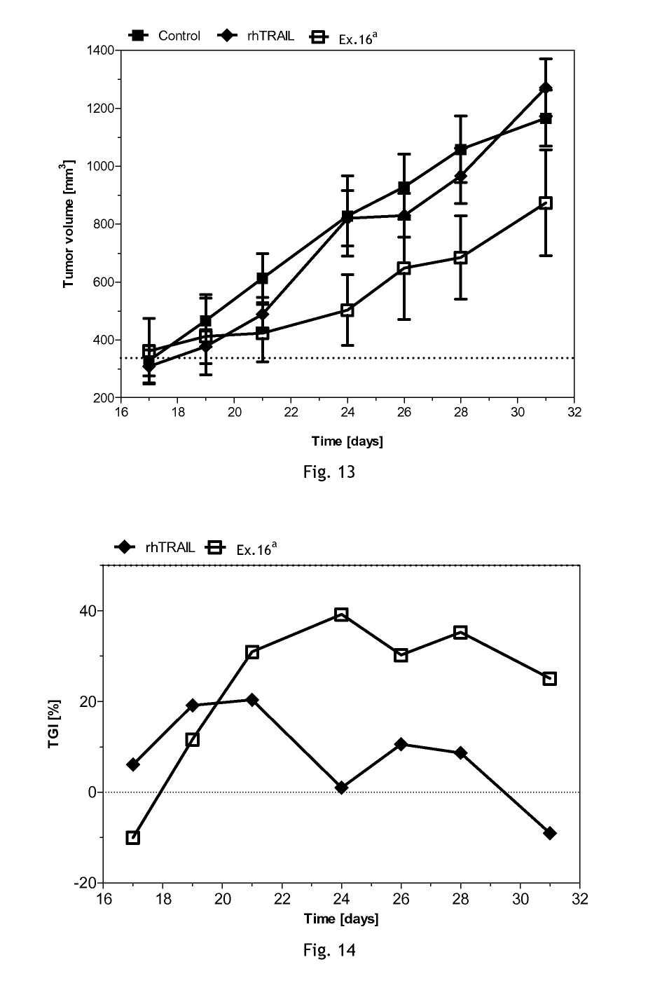

The experimental results in mice Crl:SHO-Prkdc^{scid}Hr^{hr} burdened with SW620 treated with fusion protein of the invention of Ex. 16^{a} and comparatively with rhTRAIL114-281 are shown on FIG. 13 and FIG. 14 as a diagram of changes of the tumor volume and tumor growth inhibition (% TGI) as a percentage of control.

The experimental results presented in FIGS. 13 and 14 show that administration of the fusion protein of the invention of Ex. 16^{a} caused tumor SW620 growth inhibition, with TGI equal to 25% comparing to control on the 31 day of experiment. For rhTRAIL114-281 used as the comparative reference, no inhibitory effect on tumor cell growth was obtained relative to the control, with TGI at the level of −9%. Thus, fusion proteins of the invention exerted much stronger effect compared to TRAIL alone.

The tested fusion protein did not cause significant side effects manifested by a decrease in body weight of mice (i.e. less than 10% of the baseline body weight). This shows low systemic toxicity of the protein of the invention.

### Experiment C on Colo205 Cells

On day 0 Crl:SHO-Prkdc^{scid}Hr^{hr} mice were grafted subcutaneously (s.c.) in the right side with 5×10⁶ of Colo205 cells suspended in 0.1 ml of HBSS buffer using syringe with a needle 0.5×25 mm (Bogmark). On the 13 day of the experiment mice were randomized to obtain the average size of tumors in the group of ˜115 mm³ and assigned to treatment groups.

The treatment groups were administered with the preparations of fusion protein of the invention of Ex. 16^{a} (30 mg/kg), and rhTRAIL114-281 (30 mg/kg) as a comparison against formulation buffer (19 mM NaH₂PO₄, 81 mM Na₂HPO₄, 50 mM NaCl, 5 mM glutathione, 0.1 mM ZnCl₂, 10% glycerol, pH 7.4) as a control. The preparations were administered intravenously (i.v) 6 times once daily every second day. On the 33 day of the experiment mice were sacrificed by disruption of spinal cord.

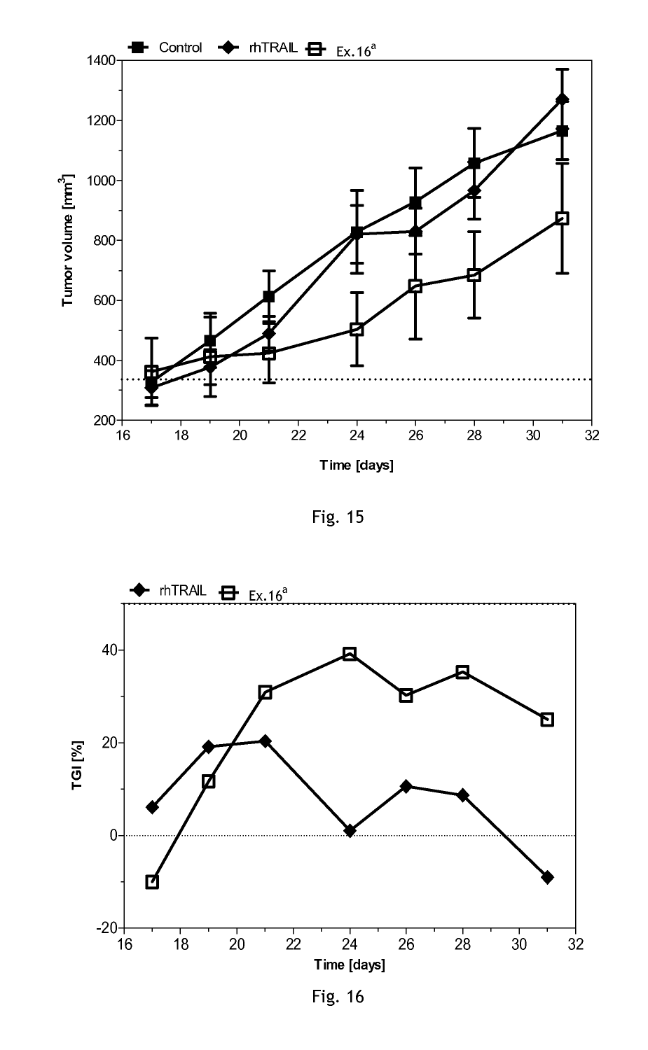

The experimental results are shown on FIG. 15 and FIG. 16 as a diagram of changes of the tumor volume and tumor growth inhibition (% TGI) as the percentage of control.

The experimental results presented in FIGS. 15 and 16 show that administration of the fusion protein of the invention of Ex. 16^{a} caused Colo205 tumor growth inhibition, with TGI equal to 80% relative to the control on 33 day of experiment. For rhTRAIL114-281 used as the comparative reference, a slight inhibitory effect on tumor cell growth was obtained relative to the control, with TGI at the level of 18.8%. Thus, fusion proteins of the invention exerted much stronger effect compared to TRAIL alone.

The tested fusion proteins did not cause significant side effects manifested by a decrease in body weight of mice (i.e. less than 10% of the baseline body weight). This shows low systemic toxicity of the protein of the invention.

### Multidrug Resistant Uterine Sarcoma Model

On day 0 Crl:SHO-Prkdc^{scid}Hr^{hr} mice were grafted subcutaneously (s.c.) in the right side with 7×10⁶ of MES-SA/Dx5 cells suspended in 0.1 ml of the mixture HBSS:Matrigel 10:1 using syringe with a needle 0.5×25 mm (Bogmark). On the 19 day of the experiment mice were randomized to obtain the average size of tumors in the group of −180 mm³ and assigned to treatment groups. The treatment groups were administered with the preparation of fusion protein of the invention of Ex. 11^{a} (30 mg/kg) and rhTRAIL114-281 (10 mg/kg) as a comparison against formulation buffer (19 mM NaH₂PO₄, 81 mM Na₂HPO₄, 50 mM NaCl, 5 mM glutathione, 0.1 mM ZnCl₂, 10% glycerol, pH 7.4) as a control. Preparations were administered intravenously (i.v.) 6 times once daily every second day. On the 35 day of the experiment mice were sacrificed by disruption of spinal cord.

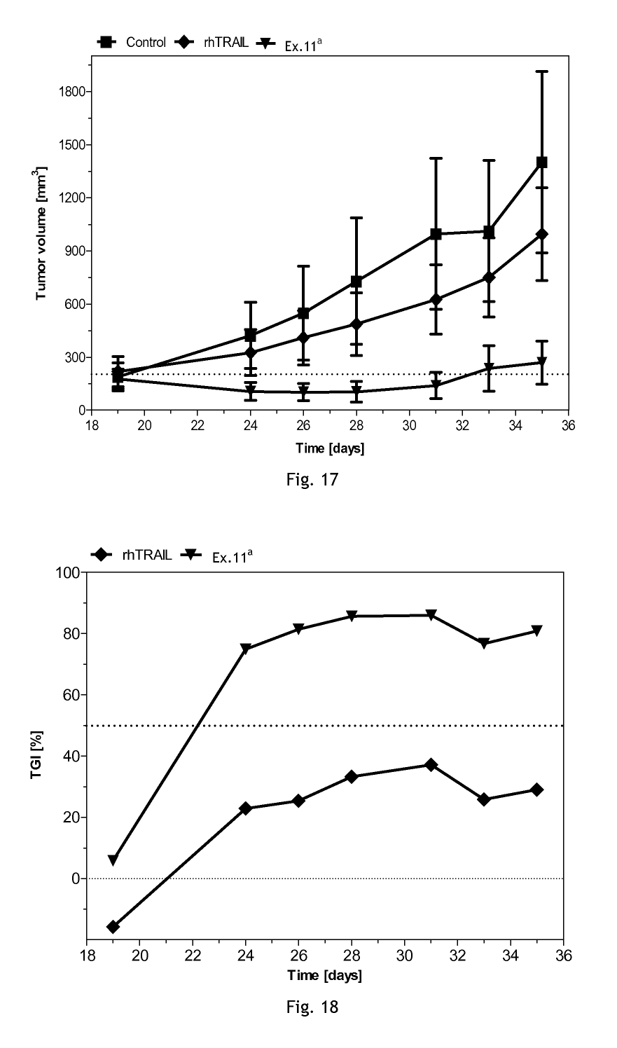

The experimental results are shown on FIG. 17 and FIG. 18 as a diagram of changes of the tumor volume and tumor growth inhibition (% TGI) as a percentage of control.

The experimental results presented in FIGS. 17 and 18 show that administration of the fusion protein of the invention of Ex. 11^{a} caused MES-SA/Dx5 tumor growth inhibition, with TGI equal to 81% relative to the control on 35 day of experiment. For rhTRAIL114-281 used as the comparative reference, a slight inhibitory effect on tumor cell growth was obtained relative to the control, with TGI at the level of 29%. Thus, fusion protein of the invention exerted much stronger effect compared to TRAIL alone.

The tested fusion protein did not cause significant side effects manifested by a decrease in body weight of mice (i.e. less than 10% of the baseline body weight). This shows low systemic toxicity of the protein of the invention.

### Liver Cancer Model

### Experiment A on HepG2 Cells

On day 0 Crl:SHO-Prkdc^{scid}Hr^{hr} mice were grafted subcutaneously (s.c.) in the right flank region with 7×10⁶ of HepG2 cells suspended in 0.1 ml of the 3:1 mixture HBSS buffer:Matrigel using syringe with a 0.5×25 mm needle (Bogmark). When tumors reached the size of 64-529 mm³ (day 25), mice were randomized to obtain the average size of tumors in the group of −230 mm³ and assigned to treatment groups. The treatment groups were administered with the preparations of fusion protein of the invention of Ex. 16^{b} (80 mg/kg) and rhTRAIL114-281 (50 mg/kg) as a comparison and reference compound 5-FU (5-Fluorouracil, Sigma-Aldrich) (20 mg/kg). The preparations were administered intravenously (i.v.) six times every second day, 5-FU was applied intraperitoneally (i.p.). The control group received formulation buffer.

When the therapeutic group reached the average tumor size of ˜1000 mm³, mice were sacrificed by cervical dislocation.

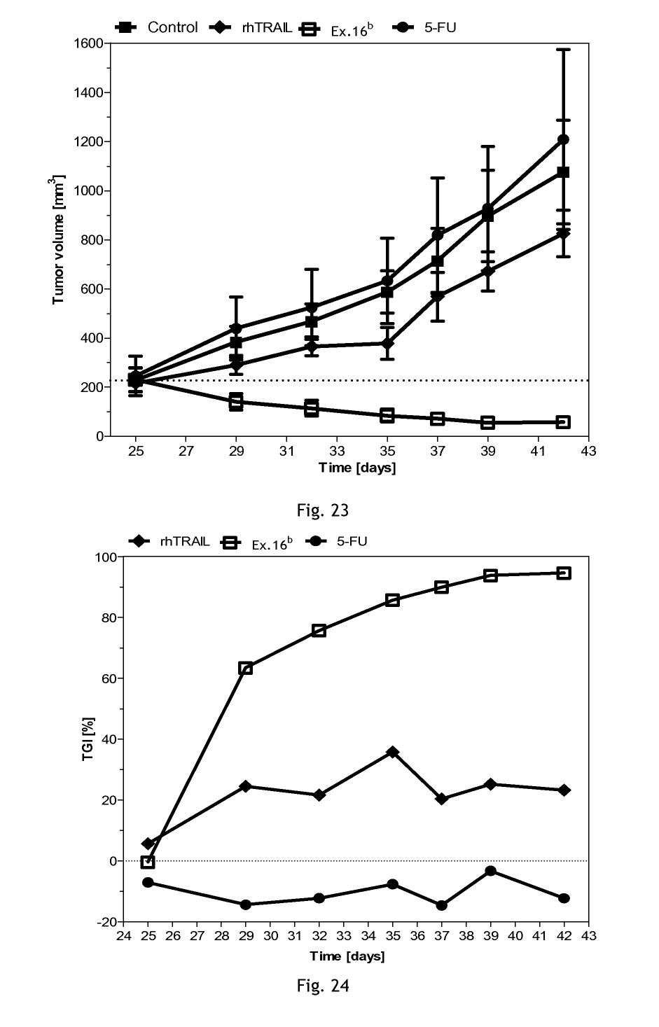

The experimental results obtained are shown in FIG. 23 as a diagram of changes of the tumor volume and in FIG. 24 which shows tumor growth inhibition (% TGI) as a percentage of control.

The results of the experiment presented in FIGS. 23 and 24 show that administration of the fusion protein of the invention of Ex. 16^{b} caused HepG2 tumor growth inhibition, with TGI 94.6% relative to the control on 42^{th} day of the experiment. For rhTRAIL114-281 as the comparative reference, a slight inhibitory effect on tumor cell growth was obtained relative to the control, with TGI at the level of 23.2%. Reference compound, 5-FU, didn't show any efficacy against HepG2 tumors. Thus, fusion protein of the invention exerted much stronger effect compared to TRAIL alone and standard chemotherapy.

### Experiment B on PLC/PRF/5 Cells

On day 0 Crl:SHO-Prkdc^{scid}Hr^{hr} mice were grafted subcutaneously (s.c.) in the right flank region with 7×10⁶ of PLC/PRF/5 cells suspended in 0.1 ml of the 3:1 mixture HBSS buffer:Matrigel using syringe with a 0.5×25 mm needle (Bogmark). When tumors reached the size of 72-536 mm³ (day 29), mice were randomized to obtain the average size of tumors in the group of ˜205 mm³ and assigned to treatment groups. The treatment groups were administered with the preparations of fusion protein of the invention of Ex. 16^{b} (50 mg/kg) and rhTRAIL114-281 (50 mg/kg) as a comparison and reference compound 5-FU (5-Fluorouracil, Sigma-Aldrich) (30 mg/kg). The preparations were administered intravenously (i.v.) six times every second day, except 5-FU, which was applied intraperitoneally (i.p.) in the schedule (q1dx5)×2. The control group received formulation buffer.

When an experimental group reached the average tumor size of ˜1000 mm³, mice were sacrificed by cervical dislocation.

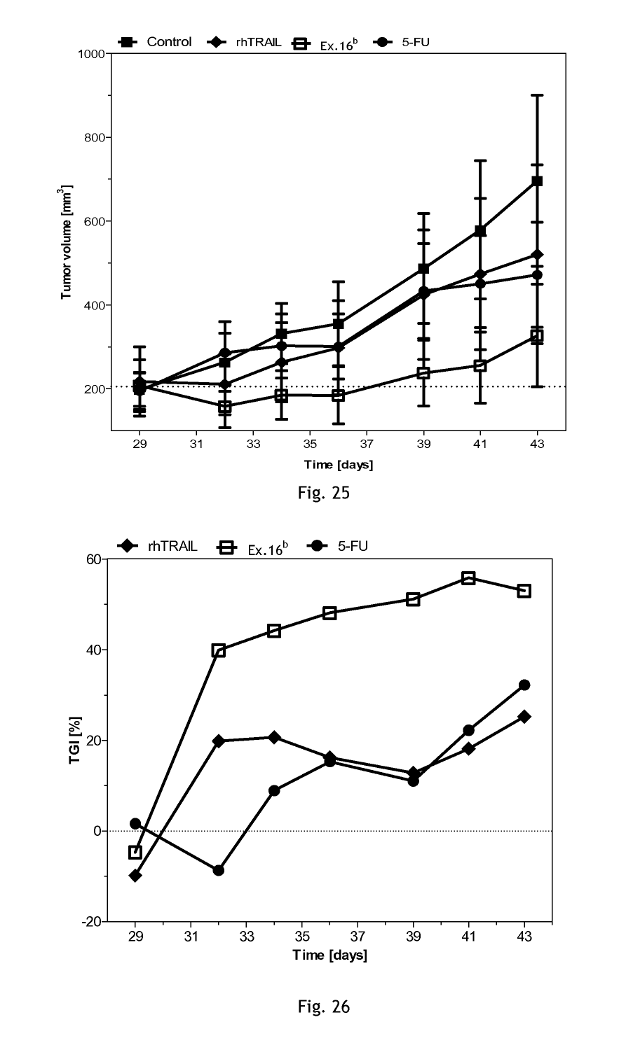

The experimental results obtained are shown in FIG. 25 as a diagram of changes of the tumor volume and in FIG. 26 which shows tumor growth inhibition (% TGI) as a percentage of control.

The results of the experiment presented in FIGS. 25 and 26 show that administration of the fusion protein of the invention of Ex. 16^{b} caused PLC/PRF/5 tumor growth inhibition, with TGI 53% relative to the control on 43^{th} day of the experiment. For rhTRAIL114-281 and 5-FU as comparative references, a slight inhibitory effect on tumor cell growth was obtained relative to the control, with TGI at the level of 25.2% and 32.2%, respectively. Thus, fusion protein of the invention exerted much stronger effect compared to TRAIL alone and standard chemotherapy.

1-41. (canceled)

42. A fusion protein comprising:
domain (a) which is a functional fragment of the sequence of soluble hTRAIL protein, which fragment begins with an amino acid at a position not lower than hTRAIL95 and ends with the amino acid at the position hTRAIL281, or a homolog of said functional fragment having at least 70% sequence identity, preferably 85% identity, and
at least one domain (b) which is the sequence of a cytolytic effector peptide forming pores in the cell membrane,
wherein the sequence of the domain (b) is attached at the C-terminus and/or N-terminus of domain (a).

43. The fusion protein according to claim 42, wherein domain (a) comprises the fragment of soluble hTRAIL (SEQ. No. 90) protein sequence, which begins with an amino acid in the range from hTRAIL95 to hTRAIL121, inclusive, and ends with the amino acid hTRAIL281.

44. The fusion protein according to claim 42, wherein domain (a) is selected from the group consisting of hTRAIL95-281, hTRAIL114-281, hTRAIL115-281, hTRAIL116-281, hTRAIL119-281, and hTRAIL121-281.

45. The fusion protein according to claim 42, wherein domain (b) is selected from the group consisting of:
active form of human granulysin of SEQ. No. 34,
15-amino acids synthetic lytic peptide of SEQ. No. 35,
pilosulin-1 of SEQ. No. 36,
pilosulin-5 of SEQ. No. 37,
peptide from tachyplesin of SEQ. No. 38,
fusion peptide bombesin-magainin 2 of SEQ. No. 39,
magainin-2 of SEQ. No. 40,
14-amino acids synthetic lytic peptide of SEQ. No. 41,
26-amino acids hybride peptide cecropin-melittin of SEQ. No. 42,
27-amino acids peptide FFhCAP18 of SEQ. No. 43,
BAMP-28 peptide of SEQ. No. 44,
analogue of isoform C of lytic peptide from Entamoeba histolytica of SEQ. No. 45,
analogue of isoform A of lytic peptide from Entamoeba histolytica SEQ. No. 46,
analogue of isoform B of lytic peptide from Entamoeba histolytica of SEQ. No. 47,
fragment of HA2 domain of influenza virus haemagglutinin of SEQ. No. 48,
N-terminal domain of alpha-toxin from Clostridium perfringens with phospholipase C activity of SEQ. No. 49,
listeriolysin O of SEQ. No. 50,
phospholipase PC-PLC of SEQ. No. 51
equinatoxin EqTx-II of SEQ. No. 52
viscotoxin A3 (VtA3) of SEQ. No. 53
active fragment of human perforin of SEQ. No. 54,
parasporin-2 z Bacillus thuringensis of SEQ. No. 55, i
fusion peptide comprising an EGF inhibitor and synthetic lytic peptide of SEQ. No. 56,
fusion protein comprising synthetic lytic peptide with KLLK motif and a peptide being antagonist of PDGF receptor of SEQ. No. 125,
pleurocidin analogue of SEQ. No. 126,
pleurocidin analogue of SEQ. No. 127,
synthetic lytic peptide of SEQ. No. 128,
fusion peptide comprising bombesin and B27 peptide of SEQ. No. 129,
17-amino acids synthetic B27 peptide of SEQ. No. 130,
fusion peptide comprising bombesin and B28 peptide of SEQ. No. 131, and
melittin peptide of SEQ. No. 132.

46. The fusion protein according to claim 42, in which domain (b) is a peptide with a strong positive charge selected from the group consisting of:
active form of human granulysin of SEQ. No. 34,
15-amino acids synthetic lytic peptide of SEQ. No. 35,
peptide from tachyplesin of SEQ. No. 38,
fusion peptide bombesin-magainin 2 of SEQ. No. 39,
magainin-2 of SEQ. No. 40,
26-amino acids hybride peptide cecropin-melittin of SEQ. No. 42,
viscotoxin A3 (VtA3) of SEQ. No. 53,
fusion peptide comprising an EGF inhibitor and synthetic lytic peptide of SEQ. No. 56,
a fusion peptide comprising bombesin and B27 peptide of SEQ. No. 129,
17-amino acids synthetic B27 peptide of SEQ. No. 130,
a fusion peptide comprising bombesin and B28 peptide of SEQ. No. 131, and
melittin peptide of SEQ. No. 132.

47. The fusion protein according to claim 42, in which domain (b) is a peptide with amphipathic alpha-helix selected from the group consisting of:
pilosulin-1 of SEQ. No. 36,
pilosulin-5 of SEQ. No. 37,
14-amino acids synthetic lytic peptide of SEQ. No. 41,
27-amino acids peptide FFhCAP18 of SEQ. No. 43,
BAMP-28 peptide of SEQ. No. 44,
analogue of isoform C of lytic peptide from Entamoeba histolytica of SEQ. No. 45,
analogue of isoform A of lytic peptide from Entamoeba histolytica SEQ. No. 46,
analogue of isoform B of lytic peptide from Entamoeba histolytica of SEQ. No. 47,
fragment of HA2 domain of influenza virus haemagglutinin of SEQ. No. 48,
active fragment of human perforin of SEQ. No. 54,
parasporin-2 z Bacillus thuringensis of SEQ. No. 55,
fusion protein comprising synthetic lytic peptide with KLLK motif and a peptide being antagonist of PDGF receptor of SEQ. No. 125,
pleurocidin analogue of SEQ. No. 126,
pleurocidin analogue of SEQ. No. 127, and
synthetic lytic peptide of SEQ. No. 128.

48. The fusion protein according to claim 42, in which domain (b) is a peptide with enzymatic activity selected from the group of phospholipase, hemolysin and/or cytolysin, preferably selected from the group consisting of:
N-terminal domain of alpha-toxin from Clostridium perfringens with phospholipase C activity of SEQ. No. 49,
listeriolysin O of SEQ. No. 50,
phospholipase PC-PLC of SEQ. No. 51, and
equinatoxin EqTx-II of SEQ. No. 52.

49. The fusion protein according to claim 42, which between domain (a) and domain (b) or between domains (b) contains domain (c) containing protease cleavage site, selected from a sequence recognized by metalloprotease MMP, a sequence recognized by urokinase uPA, and sequence recognized by furin and a sequence recognized by native furin.

50. The fusion protein according to claim 49, in which a sequence recognized by metalloprotease MMP is Pro Leu Gly Leu Ala Gly, a sequence recognized by urokinase uPA is Arg Val Val Arg, a sequence recognized by furin is Arg Lys Lys Arg, and a sequence recognized by native furin is Arg His Arg Gln Pro Arg Gly Trp Glu Gln Leu or His Arg Gln Pro Arg Gly Trp Glu Gln.

51. The fusion protein according to claim 49, in which domain (c) is a combination of sequence recognized by metalloprotease MMP and a sequence recognized by urokinase uPA located next to each other.

52. The fusion protein according to claim 42, in which effector peptide of domain (b) is additionally connected with transporting domain (d), selected from the group consisting of:
(d1) polyhistidine sequence transporting through the cell membrane comprising 6, 7, 8, 9, 10 or 11 His residues, and
(d2) polyarginine sequence transporting through a cell membrane, consisting of 6, 7, 8, 9, 10 or 11 Arg residues,
(d3) PD4 transporting sequence (protein transduction domain 4) Tyr Ala Arg Ala Ala Ala Arg Gln Ala Arg Ala,
(d4) a transporting sequence consisting of transferrin receptor binding sequence Thr His Arg Pro Pro Met Trp Ser Pro Val Trp Pro, and
(d5) PD5 transporting sequence (protein transduction domain 5, TAT protein) Tyr Gly Arg Lys Lys Arg Arg Gln Arg Arg Arg,
and combinations thereof.

53. The fusion protein according to claim 52, wherein transporting domain (d) is located between domain (b) and domain (c), or between domain (a) and domain (c), or between two domains (c).

54. The fusion protein according to claim 52, wherein sequence (d) is located at the C-terminus of the fusion protein.

55. The fusion protein according to claim 52, which between two (c) domains contains domain (d) which is a linker for attachment of PEG molecule, selected from Ala Ser Gly Cys Gly Pro Glu Gly and Ala Ser Gly Cys Gly Pro Glu.

56. The fusion protein according to claim 49, which additionally comprises a flexible steric linker between domains (a), (b) and/or (c).

57. The fusion protein according to claim 56, wherein the steric linker is selected from Gly Gly, Gly Gly Gly, Gly Ser Gly, Gly Gly Gly Gly Ser, Gly Gly Gly Gly Gly Ser, Gly Gly Ser Gly Gly, Gly Gly Gly Ser Gly Gly Gly, Gly Gly Gly Gly Ser Gly, Gly Gly Gly Ser Gly Gly Gly Gly Gly Ser, Gly Gly Gly Gly Ser Gly Gly Gly Gly, Gly Ser Gly Gly Gly Ser Gly Gly Gly, Cys Ala Ala Cys Ala Ala Ala Cys, Cys Ala Ala Ala Cys Ala Ala Cys, Ser Gly Gly, single glycine residue Gly, and single cysteine residue Cys, and combinations thereof.

58. The fusion protein according to claim 42, having the amino acid sequence selected from the group consisting of SEQ. No. 1; SEQ. No. 2; SEQ. No. 3; SEQ. No. 4; SEQ. No. 5; SEQ. No. 6; SEQ. NO. 7; SEQ. No. 8; SEQ. No. 9; SEQ. No. 10; SEQ. No. 11; SEQ. NO. 12; SEQ. No. 13; SEQ. No. 14; SEQ. No. 15; SEQ. NO. 16; SEQ. No. 17; SEQ. No. 18; SEQ. No. 19; SEQ. No. 20; SEQ. No. 21; SEQ. No. 22; SEQ. No. 23; SEQ. No. 24; SEQ. No. 25; SEQ. No. 26, SEQ. No. 27; SEQ. NO. 28; SEQ. No. 29; SEQ. NO. 30; SEQ. No. 31; SEQ. No. 32, SEQ. No. 33; SEQ. No. 91; SEQ. No. 92; SEQ. No. 93; SEQ. NO. 94; SEQ. NO. 95; SEQ. No. 96; SEQ. NO. 97, SEQ. NO. 98; SEQ. NO. 99; SEQ. NO. 100; SEQ. No. 101; SEQ. No. 102; SEQ. No. 103, SEQ. No. 104; SEQ. No. 105; SEQ. No. 106, and SEQ. No. 107.

19-25. (canceled)

59. A pharmaceutical composition comprising as an active ingredient the fusion protein as defined in claim 42, in combination with a pharmaceutically acceptable carrier.

60. A method of treating cancer diseases in mammal, including human, which comprises administration to a subject in a need thereof an anti-neoplastic-effective amount of the fusion protein as defined in claim 42, or a pharmaceutical composition.

61. Peptide selected from the group consisting of a fusion peptide comprising an EGF inhibitor and synthetic lytic peptide of SEQ. No. 56 and a fusion variant of PDGF antagonist and synthetic lytic peptide of SEQ. No. 125.


SEQUENCE LISTING (132 sequences)

SEQ ID NO: 1 - protein, 258 aa, Artificial Sequence [sequence omitted]

SEQ ID NO: 2 - protein, 261 aa, Artificial Sequence [sequence omitted]

SEQ ID NO: 3 - protein, 186 aa, Artificial Sequence
  synthesized, fusion protein comprising: a fragment of TRAIL protein, synthetic 15-amino acids lytic peptid and cleavage site sequences recognized by urokinase and metalloprotease.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGPLGLAGRVVRKLLLRLLKKLLRLLK

SEQ ID NO: 4 - protein, 227 aa, Artificial Sequence
  synthesized, fusion protein comprising: a fragment of TRAIL protein, pilosulin-1 peptide and cleavage site sequences recognized by urokinase and metalloprotease.
GLGSVFGRLARILGRVIPKVAKKLGPKVAKVLPKVMKEAIPMAVEMAKSQEEQQPQRVVRPLGLAGRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 5 - protein, 264 aa, Artificial Sequence [sequence omitted]

SEQ ID NO: 6 - protein, 299 aa, Artificial Sequence
  synthesized, fusion protein comprising: a fragment of TRAIL protein, pilosulin-5 peptide, sequences of steric linker and cleavage site sequences recognized by urokinase and metalloprotease.
TSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGSGCAACAAACPLGLAGRVVRGKLSCLSLALAIILILAIVHSPNMEVKALADPEADAFGEANAFGEADAFAEANADVKGMKKAIKEILDCVIEKGYDKLAAKLKKVIQQLWE

SEQ ID NO: 7 - protein, 224 aa, Artificial Sequence
  synthesized, fusion protein comprising: a fragment of TRAIL protein, tachyplesin peptide, sequences of steric linker and cleavage site sequences recognized by urokinase and metalloprotease.
TSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGCAACAAACGGPLGLAGRVVRKWCFRVCYRGICYRRCR

SEQ ID NO: 8 - protein, 202 aa, Artificial Sequence
  synthesized, fusion protein comprising: a fragment of TRAIL protein, tachyplesin peptide, cleavage site sequences recognized by urokinase and metalloprotease and arginine transporting sequence.
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGPLGLAGRVVRRRRRRRRKWCFRVCYRGICYRRCR

SEQ ID NO: 9 - protein, 243 aa, Artificial Sequence [sequence omitted]

SEQ ID NO: 10 - protein, 196 aa, Artificial Sequence
  synthesized, fusion protein comprising: a fragment of TRAIL protein, magainin-2 peptide and cleavage site sequences recognized by urokinase and metalloprotease.
GIGKFLHSAKKFGKAFVGEIMNSRVVRPLGLAGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 11 - protein, 202 aa, Artificial Sequence
  synthesized, fusion protein comprising: a fragment of TRAIL protein, synthetic lytic peptide, a sequence of steric linker, cleavage site sequences recognized by urokinase and metalloprotease and arginine transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGRVVRPLGLAGRRRRRRRRKLAKLAKKLAKLAK

SEQ ID NO: 12 - protein, 205 aa, Artificial Sequence
  synthesized,fusion protein comprising: a fragment of TRAIL protein, synthetic lytic peptide, sequences of steric linker, cleavage sites recognized by proteases, pegylation linker and a transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGASGCGPEGGGGPLGLAGRVVRRRRRRRRKLAKLAKKLAKLAK

SEQ ID NO: 13 - protein, 228 aa, Artificial Sequence
  synthesized, fusion protein comprising: a fragment of TRAIL protein, synthetic lytic peptide, a sequence of steric linker, cleavage site sequences recognized by urokinase and metalloprotease and arginine transporting sequence.
TSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGRVVRPLGLAGRRRRRRRRKLAKLAKKLAKLAK

SEQ ID NO: 14 - protein, 192 aa, Artificial Sequence
  synthesized, fusion protein comprising: a fragment of TRAIL protein, synthetic lytic peptide, a sequence of steric linker, cleavage site sequences recognized by urokinase and metalloprotease and histidine transporting sequence.
HHHHHHKLAKLAKKLAKLAKCRVVRPLGLAGRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 15 - protein, 200 aa, Artificial Sequence
  synthesized, fusion protein comprising: a fragment of TRAIL protein, synthetic lytic peptide, a sequence of steric linker, cleavage site sequences recognized by urokinase and metalloprotease and transporting sequences.
HHHHHHKLAKLAKKLAKLAKCRRRRRRRRRVVRPLGLAGRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 16 - protein, 202 aa, Artificial Sequence
  synthesized, fusion protein comprising: a fragment of TRAIL protein, synthetic lytic peptide, a sequence of steric linker, cleavage site sequences recognized by urokinase and metalloprotease and arginine transporting sequence.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGPLGLAGRVVRRRRRRRRRKLAKLAKKLAKLAK

SEQ ID NO: 17 - protein, 208 aa, Artificial Sequence
  synthesized, fusion protein comprising: a fragment of TRAIL protein, hybride peptide cecropin A-melittin and cleavage site sequences recognized by urokinase and metalloprotease.
KWKLFKKIGIGAVLKVLTTGLPALISRVVRPLGLAGERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGPLGLAG

SEQ ID NO: 18 - protein, 203 aa, Artificial Sequence
  synthesized, fusion protein comprising: a fragment of TRAIL protein, peptide hCAP-18/LL-37 and cleavage site sequences recognized by urokinase and metalloprotease.
FRKSKEKIGKFFKRIVQRIFDFLRNLVRVVRPLGLAGERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 19 - protein, 203 aa, Artificial Sequence
  synthesized, fusion protein comprising: a fragment of TRAIL protein, peptide BAMP-28 and cleavage site sequences recognized by urokinase and metalloprotease.
GGLRSLGRKILRAWKKYGPIIVPIIRIRVVRPLGLAGERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 20 - protein, 200 aa, Artificial Sequence
  synthesized, fusion protein comprising: a fragment of TRAIL protein, analogue of isoform C of the lytic peptide from Entamoeba histolytica, steric linker and cleavage site sequences recognized by urokinase and metalloprotease.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGSGGPLGLAGRVVRGLVETLTKIVSYGIDKLIEKILEG

SEQ ID NO: 21 - protein, 200 aa, Artificial Sequence
  synthesized, fusion protein comprising: a fragment of TRAIL protein, analogue of isoform A of the lytic peptide from Entamoeba histolytica, steric linker and cleavage site sequences recognized by urokinase and metalloprotease.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGSGGPLGLAGRVVRGFIATLTKVLDFGIDKLIQLIEDK

SEQ ID NO: 22 - protein, 200 aa, Artificial Sequence
  synthesized, fusion protein comprising: a fragment of TRAIL protein, analogue of isoform B of the lytic peptide from Entamoeba histolytica, steric linker and cleavage site sequences recognized by urokinase and metalloprotease.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGSGGPLGLAGRVVRGFLGTLEKILSFGVDELVKLIENH

SEQ ID NO: 23 - protein, 190 aa, Artificial Sequence
  synthesized, fusion protein comprising: a fragment of TRAIL protein, fragment of HA2 domain of influenza virus haemagglutinin, arginine transporting sequence and cleavage site sequences recognized by urokinase and metalloprotease.
GLLEALAELLEGRRRRRRRRVVRPLGLAGRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 24 - protein, 429 aa, Artificial Sequence
  synthesized, fusion protein comprising: a fragment of TRAIL protein, a domain of alpha-toxin from Clostridium perfringens, sequences of steric linkers and pegylation linker.
WDGKIDGTGTHAMIVTQGVSILENDMSKNEPESVRKNLEILKDNMHELQLGSTYPDYDKNAYDLYQDHFWDPDTNNNFSKDNSWYLAYSIPDTGESQIRKFSALARYEWQRGNYKQATFYLGEAMHYFGDIDTPYHPANVTAVDSAGHVKFETFAEERKEQYKINTVGCKTNEDFYADILKNKDFNAWSKEYARGFAKTGKSIYYSHASMSHSWDDWDYAAKVTLANSQKGTAGYIYRFLHDVSEGNGGGGGGSASGCGPEGGGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 25 - protein, 658 aa, Artificial Sequence
  synthesized, fusion protein comprising: a fragment of TRAIL protein, listeriolysin O peptide, sequences of steric linkers, furin cleavage site and pegylation linker.
HADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEINYDPEGNEIVQHKNWSENNKSKLAHFTSSIYLPGNARNINVYAKECTGLAWEWWRTVIDDRNLPLVKNRNISIWGTTLYPKYSNSVGGGGSGGGGGSRKKRASGCGPEGGGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 26 - protein, 478 aa, Artificial Sequence
  synthesized, fusion protein comprising: a fragment of TRAIL protein, phospholipase PC-PLC peptide, sequences of steric linkers, furin cleavage site and pegylation linker.
KFKKVVLGMCLTASVLVFPVTIKASACCDEYLKPPAAPHDIDSKLPHKLSWSADNPTNTDVNTHYWLFKQAEKILAKDVDHMRANLMNELKNFDKQIAQGIYDADHKNPYYDTSTFLSHFYNPDKDNTYLPGFANAKITGAKYFNQSVADYREGKFDTAFYKLGLAIHYYTDISQPMHANNFTAISYPPGYHCAYENYVDTIKHNYQATEDMVVQRFCSNDVKEWLYENAKRAKADYPKIVNAKTKKSYLVGNSEWKKDTVEPTGARLRDSQQTLAGFLEFWSKKTNEGGGGSGGGGGSRKKRASGCGPEGGGGGGSRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 27 - protein, 361 aa, Artificial Sequence [sequence omitted]

SEQ ID NO: 28 - protein, 227 aa, Artificial Sequence [sequence omitted]

SEQ ID NO: 29 - protein, 224 aa, Artificial Sequence
  synthesized, fusion protein comprising: a fragment of TRAIL protein, viscotoxin A3 peptide, a sequence of steric linker and urokinase and metalloprotease cleavage sites.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGGPLGLAGRVVRKSCCPNTTGRNIYNACRLTGAPRPTCAKLSGCKIISGSTCPSDYPK

SEQ ID NO: 30 - protein, 200 aa, Artificial Sequence
  synthesized, fusion protein comprising: a fragment of TRAIL protein, human perforin peptide and a sequence of steric linker.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGAPCHTAARSECKRSHKFVPGAWLAGEGVDVTSL

SEQ ID NO: 31 - protein, 210 aa, Artificial Sequence
  synthesized, fusion protein comprising: a fragment of TRAIL protein, human perforin peptide, cleavage sites recognized by urokinase and metalloproteinase and a steric linker.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGRVVRPLGLAGAPCHTAARSECKRSHKFVPGAWLAGEGVDVTSL

SEQ ID NO: 32 - protein, 436 aa, Artificial Sequence [sequence omitted]

SEQ ID NO: 33 - protein, 215 aa, Artificial Sequence [sequence omitted]

SEQ ID NO: 34 - protein, 83 aa, Homo sapiens
  ref <308>: DATABASE ACCESSION NUMBER: GenBank/AAH23576.1
  ref <309>: DATABASE ENTRY DATE: 2006-07-15
  ref <313>: RELEVANT RESIDUES IN SEQ ID NO: (63)..(145)
GRDYRTCLTIVQKLKKMVDKPTQRSVSNAATRVCRTGRSRWRDVCRNFMRRYQSRVTQGLVAGETAQQICEDLRLCIPSTGPL

SEQ ID NO: 35 - protein, 15 aa, Artificial Sequence
  synthesized efector peptide
  ref <301>: AUTHORS: Papo N, Shahar M, Eisenbach L, Shai Y.
  ref <302>: TITLE: A novel lytic peptide composed of DL-amino acids selectively kills cancer cells in culture and in mice.
  ref <303>: JOURNAL: J BiolChem
  ref <304>: VOLUME: 278
  ref <305>: ISSUE: 3
  ref <306>: PAGES: 21018-23
  ref <307>: DATE: 2003-06-06
KLLLRLLKKLLRLLK

SEQ ID NO: 36 - protein, 56 aa, Myrmecia pilosula
  ref <308>: DATABASE ACCESSION NUMBER: Swiss-Prot/Q07932.1
  ref <309>: DATABASE ENTRY DATE: 2011-01-11
  ref <313>: RELEVANT RESIDUES IN SEQ ID NO: (57)..(112)
GLGSVFGRLARILGRVIPKVAKKLGPKVAKVLPKVMKEAIPMAVEMAKSQEEQQPQ

SEQ ID NO: 37 - protein, 90 aa, Myrmecia banksi
  ref <308>: DATABASE ACCESSION NUMBER: GenBank/BAF95069.1
  ref <309>: DATABASE ENTRY DATE: 2008-08-04
  ref <313>: RELEVANT RESIDUES IN SEQ ID NO: (2)..(91)
KLSCLSLALAIILILAIVHSPNMEVKALADPEADAFGEANAFGEADAFAEANADVKGMKKAIKEILDCVIEKGYDKLAAKLKKVIQQLWE

SEQ ID NO: 38 - protein, 17 aa, Tachypleus tridentatus
  ref <308>: DATABASE ACCESSION NUMBER: GenBank/AAA63538.1
  ref <309>: DATABASE ENTRY DATE: 1995-03-07
  ref <313>: RELEVANT RESIDUES IN SEQ ID NO: (24)..(40)
KWCFRVCYRGICYRRCR

SEQ ID NO: 39 - protein, 38 aa, Artificial Sequence
  synthesized efector peptide
  ref <301>: AUTHORS: Liu S, Yang H, Wan L, Cai HW, Li SF, Li YP, Cheng JQ, Lu XF.
  ref <302>: TITLE: Enhancement of cytotoxicity of antimicrobial peptide magainin II in tumor cells by bombesin-targeted delivery
  ref <303>: JOURNAL: Acta Pharmacol Sin
  ref <304>: VOLUME: 32
  ref <305>: ISSUE: 1
  ref <306>: PAGES: 79-88
  ref <307>: DATE: 2011-01-01
GIGKFLHSAKKFGKAFVGEIMNSGGQRLGNQWAVGHLM

SEQ ID NO: 40 - protein, 23 aa, Xenopus laevis
  ref <308>: DATABASE ACCESSION NUMBER: Swiss-Prot/P11006.1
  ref <309>: DATABASE ENTRY DATE: 2010-11-30
  ref <313>: RELEVANT RESIDUES IN SEQ ID NO: (83)..(105)
GIGKFLHSAKKFGKAFVGEIMNS

SEQ ID NO: 41 - protein, 14 aa, Artificial Sequence
  synthesized efector peptide
  ref <301>: AUTHORS: avadpour MM, Juban MM, Lo WC, Bishop SM, Alberty JB, Cowell SM, Becker CL, McLaughlin ML
  ref <302>: TITLE: De novo antimicrobial peptides with low mammalian cell toxicity
  ref <303>: JOURNAL: J Med Chem
  ref <304>: VOLUME: 39
  ref <305>: ISSUE: 16
  ref <306>: PAGES: 3107-13
  ref <307>: DATE: 1996-08-02
KLAKLAKKLAKLAK

SEQ ID NO: 42 - protein, 26 aa, Artificial Sequence
  synthesized efector peptide
  ref <301>: AUTHORS: M., Velasco, M., J., Diaz-Guerra, P., Diaz-Achirica, D., Andreu, L., Rivas and L., Bosca,
  ref <302>: TITLE: Macrophage triggering with cecropin A and melittin-derived peptides induces type II nitric oxide synthase expression
  ref <303>: JOURNAL: The Journal of Immunology
  ref <304>: VOLUME: 158
  ref <305>: ISSUE: 9
  ref <306>: PAGES: 4437-4443
  ref <307>: DATE: 1997-09-13
KWKLFKKIGIGAVLKVLTTGLPALIS

SEQ ID NO: 43 - protein, 27 aa, Artificial Sequence
  synthesized efector peptide
  ref <301>: AUTHORS: Isogai E.
  ref <302>: TITLE: Antimicrobial and Lipopolysaccharide-Binding Activities of C-Terminal Domain of Human CAP18 Peptides to Genus Leptospira
  ref <303>: JOURNAL: The Journal of Applied Research
  ref <304>: VOLUME: 4
  ref <305>: ISSUE: 1
  ref <306>: PAGES: 180-185
  ref <307>: DATE: 2004-12-01
FRKSKEKIGKFFKRIVQRIFDFLRNLV

SEQ ID NO: 44 - protein, 27 aa, Bos taurus
  ref <301>: AUTHORS: Risso A, Braidot E, Sordano MC, Vianello A, Macr? F, Skerlavaj B, Zanetti M, Gennaro R, Bernardi P.
  ref <302>: TITLE: MAP-28, an antibiotic peptide of innate immunity, induces cell death through opening of the mitochondrial permeability transition pore.
  ref <303>: JOURNAL: Mol Cell Biol.
  ref <304>: VOLUME: 22
  ref <305>: ISSUE: 6
  ref <306>: PAGES: 1926-35
  ref <307>: DATE: 2002-03
GGLRSLGRKILRAWKKYGPIIVPIIRI

SEQ ID NO: 45 - protein, 24 aa, Entamoeba histolytica
  ref <301>: AUTHORS: Andrd J, Berninghausen O, W|lfken J, Leippe M.
  ref <302>: TITLE: Shortened amoebapore analogswith enhanced antibacterial and cytolytic activity.
  ref <303>: JOURNAL: FEBS Lett.
  ref <304>: VOLUME: 385
  ref <305>: ISSUE: 12
  ref <306>: PAGES: 96-100
  ref <307>: DATE: 1996-04-29
GLVETLTKIVSYGIDKLIEKILEG

SEQ ID NO: 46 - protein, 24 aa, Entamoeba histolytica
  ref <301>: AUTHORS: Andrd J, Berninghausen O, W|lfken J, Leippe M.
  ref <302>: TITLE: Shortened amoebapore analogswith enhanced antibacterial and cytolytic activity.
  ref <303>: JOURNAL: FEBS Lett.
  ref <304>: VOLUME: 385
  ref <305>: ISSUE: 1
  ref <306>: PAGES: 96-100
  ref <307>: DATE: 1996-04-29
GFIATLTKVLDFGIDKLIQLIEDK

SEQ ID NO: 47 - protein, 24 aa, Entamoeba histolytica
  ref <301>: AUTHORS: Andrd J, Berninghausen O, W|lfken J, Leippe M.
  ref <302>: TITLE: Shortened amoebapore analogswith enhanced antibacterial and cytolytic activity.
  ref <303>: JOURNAL: FEBS Lett.
  ref <304>: VOLUME: 385
  ref <305>: ISSUE: 1
  ref <306>: PAGES: 96-100
  ref <307>: DATE: 1996-04-29
GFLGTLEKILSFGVDELVKLIENH

SEQ ID NO: 48 - protein, 12 aa, Influenza virus
  ref <301>: AUTHORS: Ines Neundorf, Robert Rennert, Jan Hoyer, Franziska Schramm, Kristin Lvbner Igor Kitanovic  and Stefan Wvlfl
  ref <302>: TITLE: Fusion of a Short HA2-Derived Peptide Sequence to Cell-Penetrating Peptides Improves Cytosolic Uptake, but Enhances Cytotoxic Activity.
  ref <303>: JOURNAL: Pharmaceuticals
  ref <304>: VOLUME: 2
  ref <305>: ISSUE: 2
  ref <306>: PAGES: 49-65
  ref <307>: DATE: 2009
GLLEALAELLEG

SEQ ID NO: 49 - protein, 247 aa, Clostridium perfringens
  ref <308>: DATABASE ACCESSION NUMBER: GenBank/AAP15462.1
  ref <309>: DATABASE ENTRY DATE: 2003-04-27
  ref <313>: RELEVANT RESIDUES IN SEQ ID NO: (1)..(247)
WDGKIDGTGTHAMIVTQGVSILENDMSKNEPESVRKNLEILKDNMHELQLGSTYPDYDKNAYDLYQDHFWDPDTNNNFSKDNSWYLAYSIPDTGESQIRKFSALARYEWQRGNYKQATFYLGEAMHYFGDIDTPYHPANVTAVDSAGHVKFETFAEERKEQYKINTVGCKTNEDFYADILKNKDFNAWSKEYARGFAKTGKSIYYSHASMSHSWDDWDYAAKVTLANSQKGTAGYIYRFLHDVSEGN

SEQ ID NO: 50 - protein, 468 aa, Listeria monocytogenes
  ref <308>: DATABASE ACCESSION NUMBER: GenBank/AAS85208.1
  ref <309>: DATABASE ENTRY DATE: 2008-08-26
  ref <313>: RELEVANT RESIDUES IN SEQ ID NO: (57)..(524)
HADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEINYDPEGNEIVQHKNWSENNKSKLAHFTSSIYLPGNARNINVYAKECTGLAWEWWRTVIDDRNLPLVKNRNISIWGTTLYPKYSNSV

SEQ ID NO: 51 - protein, 289 aa, Listeria monocytogenes
  ref <308>: DATABASE ACCESSION NUMBER: genbank/ABG57041.1
  ref <309>: DATABASE ENTRY DATE: 2011-05-06
  ref <313>: RELEVANT RESIDUES IN SEQ ID NO: (5)..(292)
KFKKVVLGMCLTASVLVFPVTIKASACCDEYLKPPAAPHDIDSKLPHKLSWSADNPTNTDVNTHYWLFKQAEKILAKDVDHMRANLMNELKNFDKQIAQGIYDADHKNPYYDTSTFLSHFYNPDKDNTYLPGFANAKITGAKYFNQSVADYREGKFDTAFYKLGLAIHYYTDISQPMHANNFTAISYPPGYHCAYENYVDTIKHNYQATEDMVVQRFCSNDVKEWLYENAKRAKADYPKIVNAKTKKSYLVGNSEWKKDTVEPTGARLRDSQQTLAGFLEFWSKKTNEG

SEQ ID NO: 52 - protein, 179 aa, Actinia equina
  ref <308>: DATABASE ACCESSION NUMBER: PDB/1KD6_A
  ref <309>: DATABASE ENTRY DATE: 2009-07-10
  ref <313>: RELEVANT RESIDUES IN SEQ ID NO: (1)..(179)
SADVAGAVIDGASLSFDILKTVLEALGNVKRKIAVGVDNESGKTWTALNTYFRSGTSDIVLPHKVPHGKALLYNGQKDRGPVATGAVGVLAYLMSDGNTLAVLFSVPYDYNWYSNWWNVRIYKGKRRADQRMYEELYYNLSPFRGDNGWHTRNLGYGLKSRGFMNSSGHAILEIHVTKA

SEQ ID NO: 53 - protein, 46 aa, Viscum album
  ref <308>: DATABASE ACCESSION NUMBER: genBank/P01538.2
  ref <309>: DATABASE ENTRY DATE: 2011-05-31
  ref <313>: RELEVANT RESIDUES IN SEQ ID NO: (27)..(72)
KSCCPNTTGRNIYNACRLTGAPRPTCAKLSGCKIISGSTCPSDYPK

SEQ ID NO: 54 - protein, 33 aa, Homo sapiens
  ref <308>: DATABASE ACCESSION NUMBER: GenBank/CAA31612.1
  ref <309>: DATABASE ENTRY DATE: 2008-10-07
  ref <313>: RELEVANT RESIDUES IN SEQ ID NO: (21)..(53)
APCHTAARSECKRSHKFVPGAWLAGEGVDVTSL

SEQ ID NO: 55 - protein, 251 aa, Bacillus thuringiensis
  ref <308>: DATABASE ACCESSION NUMBER: PDB/2ZTB_A
  ref <309>: DATABASE ENTRY DATE: 2009-03-27
  ref <313>: RELEVANT RESIDUES IN SEQ ID NO: (2)..(251)
DVIREYLMFNELSALSSSPESVRSRFSSIYGTNPDGIALNNETYFNAVKPPITAQYGYYCYKNVGTVQYVNRPTDINPNVILAQDTLTNNTNEPFTTTITITGSFTNTSTVTSSTTTGFKFTSKLSIKKVFEIGGEVSFSTTIGTSETTTETITVSKSVTVTVPAQSRRTIQLTAKIAKESADFSAPITVDGYFGANFPKRVGPGGHYFWFNPARDVLNTTSGTLRGTVTNVSSFDFQTIVQPARSLLDEQ

SEQ ID NO: 56 - protein, 32 aa, Artificial Sequence
  synthesized efector peptide
  ref <302>: TITLE: SELECTIVE ANTICANCER CHIMERIC PEPTIDE
  ref <310>: PATENT DOCUMENT NUMBER: WO2010064207
  ref <311>: PATENT FILING DATE: 2009-12-03
  ref <312>: PUBLICATION DATE: 2010-06-10
YKWYGYTPQNVIGGGKLLLKLLKKLLKLLKKK

SEQ ID NO: 57 - DNA, 774 bp, Artificial Sequence
  synthesized, encoding fusion protein comprising: a fragment of TRAIL protein, active form of human granulysin, sequences of steric linkers and cleavage site sequences recognized by urokinase and metalloprotease.
cgtgttgcagcacatattaccggcacccgtggtcgtagcaataccctgagcagcccgaatagcaaaaatgaaaaagccctgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgacaaacaaatggtgcagtatatctacaaatacaccagctatccggatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacatctgattgatatggatcatgaagccagcttttttggtgcatttctggttggtggtggtagcggtccgctgggtctggcaggtcgtgttgttcgtggtcgtgattatcgtacctgtctgaccattgtgcagaaactgaaaaaaatggtggataaaccgacccagcgtagcgttagcaatgcagcaacccgtgtttgtcgtaccggtcgtagccgttggcgtgatgtttgtcgtaatttcatgcgtcgttatcagagccgtgttacccagggtctggttgccggtgaaaccgcacagcagatttgtgaagatctgcgtctgtgtattccgagcaccggtccgctg

SEQ ID NO: 58 - DNA, 783 bp, Artificial Sequence
  synthesized, encoding fusion protein comprising: a fragment of TRAIL protein, active form of human granulysin, a sequence of steric linkers and cleavage site sequences recognized by urokinase and metalloprotease.
ggtcgtgattatcgtacctgtctgaccattgttcagaaactgaaaaaaatggtggataaaccgacccagcgtagcgttagcaatgcagcaacccgtgtttgtcgtaccggtcgtagccgttggcgtgatgtttgtcgtaactttatgcgtcgttatcagagccgtgttacccagggtctggttgccggtgaaaccgcacagcagatttgtgaagatctgcgtctgtgtattccgagcaccggtccgctgcgtgttgttcgtccgctgggtctggcaggcggtggtggtggtagtccgcagcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaattaaagaaaataccaaaaatgataagcagatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggt

SEQ ID NO: 59 - DNA, 558 bp, Artificial Sequence
  synthesized, encoding fusion protein comprising: a fragment of TRAIL protein, synthetic 15-amino acids lytic peptid and cleavage site sequences recognized by urokinase and metalloprotease.
cgtgttgcagcacatattaccggcacccgtggtcgtagcaataccctgagcagcccgaatagcaaaaatgaaaaagccctgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatataccagctatccggatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacatctgattgatatggatcatgaagccagcttttttggtgcatttctggttggtccgctgggtctggcaggtcgtgttgttcgtaaactgctgctgcgtttactgaaaaaattactgcgcctgctgaaa

SEQ ID NO: 60 - DNA, 681 bp, Artificial Sequence
  synthesized, encoding fusion protein comprising: a fragment of TRAIL protein, pilosulin-1 peptide and cleavage site sequences recognized by urokinase and metalloprotease.
ggtctgggtagcgtttttggtcgtctggcacgtattctgggtcgtgttattccgaaagttgcaaaaaaactgggtccgaaagtggccaaagttctgccgaaagttatgaaagaagcaattccgatggcagttgaaatggcaaaaagccaagaagaacagcagccgcagcgtgttgttcgtccgctgggtctggcaggtcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgataaacaaatggtgcagtacatttacaaatataccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggtgggt

SEQ ID NO: 61 - DNA, 792 bp, Artificial Sequence
  synthesized, encoding fusion protein comprising: a fragment of TRAIL protein, pilosulin-1 peptide, a sequence of steric linker and cleavage site sequences recognized by urokinase and metalloprotease.
accagcgaagaaaccattagcaccgttcaagaaaaacagcagaatattagtccgctggttcgtgaacgtggtccgcagcgtgttgcagcacatattaccggcacccgtggtcgtagcaataccctgagcagcccgaatagcaaaaatgaaaaagccctgggtcgcaaaattaacagctgggaaagcagccgtagcggtcatagctttctgagcaatctgcatctgcgtaatggtgaactggtgattcacgagaaaggcttctattatatctatagccagacctattttcgctttcaagaagaaattaaagaaaacaccaaaaatgataaacaaatggtgcagtatatttacaaatataccagctatccggatccgattctgctgatgaaaagcgcacgtaatagctgttggagcaaagatgcagaatatggcctgtatagcatctatcagggtggcatttttgaactgaaagaaaacgatcgcatctttgtgagcgtgaccaatgaacatctgattgatatggatcacgaagccagcttttttggtgcatttctggttggttgtgcagcatgtgcagccgcatgtggtggtggtccgctgggtctggcaggtcgtgttgttcgtggtctgggtagcgtttttggtcgtctggcacgtattctgggtcgtgttattccgaaagttgcaaaaaaactgggtccgaaagtggccaaagttctgccgaaagttatgaaagaagcaattccgatggccgttgaaatggcaaaaagccaagaagaacagcagccgcag

SEQ ID NO: 62 - DNA, 897 bp, Artificial Sequence
  synthesized, encoding fusion protein comprising: a fragment of TRAIL protein, pilosulin-5 peptide, sequences of steric linker and cleavage site sequences recognized by urokinase and metalloprotease.
accagcgaagaaaccattagcaccgttcaagaaaaacagcagaatattagtccgctggttcgtgaacgtggtccgcagcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttctactatatctatagccagacctatttccgcttccaagaagaaatcaaagaaaataccaaaaatgataaacaaatggtgcagtatatttacaaatataccagctatccggatccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggtggtagcggttgtgcagcatgtgcagccgcatgtccgctgggtctggcaggtcgtgttgttcgtggtaaactgagctgtctgagcctggcactggcaattattctgattctggcaattgttcatagcccgaatatggaagttaaagcactggcagatccggaagcagatgcatttggtgaagcaaatgcctttggcgaagccgatgcgtttgccgaagccaatgcagatgttaaaggtatgaaaaaagccattaaagaaattctggattgcgtgatcgagaaaggctatgataaactggcagccaaactgaaaaaagttattcagcagctgtgggaa

SEQ ID NO: 63 - DNA, 672 bp, Artificial Sequence
  synthesized, encoding fusion protein comprising: a fragment of TRAIL protein, tachyplesin peptide, sequences of steric linker and cleavage site sequences recognized by urokinase and metalloprotease.
accagcgaagaaaccattagcaccgttcaagaaaaacagcagaatattagtccgctggttcgtgaacgtggtccgcagcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaattaaagaaaacaccaaaaatgataaacaaatggtgcagtacatttataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggttgtgcagcatgtgcagccgcatgtggtggtccgctgggtctggcaggtcgtgttgttcgtaaatggtgttttcgtgtttgctatcgcggtatttgttatcgtcgttgccgc

SEQ ID NO: 64 - DNA, 606 bp, Artificial Sequence
  synthesized, encoding fusion protein comprising: a fragment of TRAIL protein, tachyplesin peptide, cleavage site sequences recognized by urokinase and metalloprotease and arginine transporting sequence.
gttcgtgaacgtggtccgcagcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaggaaattaaagaaaataccaaaaatgataaacaaatggtgcagtacatctataaatacaccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtccgctgggtctggcaggtcgtgttgtgcgtcgtcgccgtcgccgtcgtcgtaaatggtgttttcgtgtttgttatcgcggtatttgttatcgtcgttgccgc

SEQ ID NO: 65 - DNA, 729 bp, Artificial Sequence
  synthesized, encoding fusion protein comprising: a fragment of TRAIL protein, fusion peptide bombesin-magainin, cleavage site sequences recognized by urokinase and metalloprotease and a sequence of steric linker.
ggtattggcaaatttctgcatagcgccaaaaaattcggcaaagcatttgtgggcgaaattatgaatagcggtggtcagcgtctgggtaatcagtgggcagttggtcatctgatgcgtgttgttcgtccgctgggtctggcaggttgtgcagcagcatgtgcagcctgtaccagcgaagaaaccattagcaccgttcaagaaaaacagcagaatattagtccgctggttcgtgaacgtggtccgcagcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttctactatatctatagccagacctatttccgcttccaagaagaaatcaaagaaaacaccaaaaatgataaacaaatggtgcagtatatctacaaatataccagctatccggatccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggtgggt

SEQ ID NO: 66 - DNA, 588 bp, Artificial Sequence
  synthesized, encoding fusion protein comprising: a fragment of TRAIL protein, magainin-2 peptide and cleavage site sequences recognized by urokinase and metalloprotease.
ggtattggcaaatttctgcatagcgccaaaaaatttggcaaagcatttgtgggcgaaattatgaatagccgtgttgttcgtccgctgggtctggcaggtccgcagcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaggaaattaaagaaaataccaaaaatgataaacaaatggtgcagtacatctataaatacaccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggtgggt

SEQ ID NO: 67 - DNA, 606 bp, Artificial Sequence
  synthesized, encoding fusion protein comprising: a fragment of TRAIL protein, synthetic lytic peptide, a sequence of steric linker, cleavage site sequences recognized by urokinase and metalloprotease and arginine transporting sequence.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaattaaagaaaacaccaaaaatgataaacaaatggtgcagtatatttacaaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggcggtagcggtggtggtggtcgtgttgttcgtccgctgggtctggcaggtcgtcgtcgtcgccgtcgtcggcgtaaactggcaaaactggccaaaaaactggcgaaactggctaaa

SEQ ID NO: 68 - DNA, 615 bp, Artificial Sequence
  synthesized,encoding fusion protein comprising: a fragment of TRAIL protein, synthetic lytic peptide, sequences of steric linker, cleavage sites recognized by proteases, pegylation linker and a transporting sequence.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaattaaagaaaacaccaaaaatgataaacaaatggtgcagtatatctacaaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtgcaagcggttgtggtccggaaggtggtggtggtccgctgggtctggcaggtcgtgttgttcgtcgtcgtcgtcgccgtcgccgtaaactggcaaaactggccaaaaaactggcgaaactggctaaa

SEQ ID NO: 69 - DNA, 684 bp, Artificial Sequence
  synthesized, encoding fusion protein comprising: a fragment of TRAIL protein, synthetic lytic peptide, a sequence of steric linker, cleavage site sequences recognized by urokinase and metalloprotease and arginine transporting sequence.
accagcgaagaaaccattagcaccgttcaggaaaaacagcagaatattagtccgctggttcgtgaacgtggtccgcagcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatacaccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggcggtagcggtggtggtggtcgtgttgttcgtccgctgggtctggcaggtcgtcgtcgtcgtagacgtcgtcgtaaactggcaaaactggccaaaaaactggcgaaactggctaaa

SEQ ID NO: 70 - DNA, 576 bp, Artificial Sequence
  synthesized, encoding fusion protein comprising: a fragment of TRAIL protein, synthetic lytic peptide, a sequence of steric linker, cleavage site sequences recognized by urokinase and metalloprotease and histidine transporting sequence.
catcatcatcaccatcacaaactggcaaaactggccaaaaaactggcgaaactggctaaatgtcgtgttgttcgtccgctgggtctggcaggtcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgataaacaaatggtgcagtatatctataaatataccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggtgggt

SEQ ID NO: 71 - DNA, 600 bp, Artificial Sequence
  synthesized, encoding fusion protein comprising: a fragment of TRAIL protein, synthetic lytic peptide, a sequence of steric linker, cleavage site sequences recognized by urokinase and metalloprotease and transporting sequences.
catcatcatcaccatcacaaactggcaaaactggccaaaaaactggcgaaactggctaaatgtcgtcgtcgtcgccgtcggcgtcgtcgtgttgttcgtccgctgggtctggcaggtcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgataaacaaatggtgcagtatatctataaatacaccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggtgggt

SEQ ID NO: 72 - DNA, 606 bp, Artificial Sequence
  synthesized, encoding fusion protein comprising: a fragment of TRAIL protein, synthetic lytic peptide, a sequence of steric linker, cleavage site sequences recognized by urokinase and metalloprotease and arginine transporting sequence.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatacaccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggcggtagcggtggtggtggtcgtgttgttcgtccgctgggtctggcaggtcgtcgtcgtcgtagacgtcgtcgtaaactggcaaaactggccaaaaaactggcgaaactggctaaa

SEQ ID NO: 73 - DNA, 624 bp, Artificial Sequence
  synthesized, encoding fusion protein comprising: a fragment of TRAIL protein, hybride peptide cecropin A-melittin and cleavage site sequences recognized by urokinase and metalloprotease.
aaatggaaactgtttaaaaaaattggcattggtgccgttctgaaagttctgaccaccggtctgcctgcactgattagccgtgttgttcgtccgctgggtctggcaggcgaacgtggtccgcagcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaggaaattaaagaaaataccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtccgctgggcctggctggt

SEQ ID NO: 74 - DNA, 609 bp, Artificial Sequence
  synthesized, encoding fusion protein comprising: a fragment of TRAIL protein, peptide hCAP-18/LL-37 and cleavage site sequences recognized by urokinase and metalloprotease.
tttcgcaaaagcaaagaaaaaattggcaaattttttaaacgcattgtgcagcgcatttttgattttctgcgtaatctggttcgtgttgttcgtccgctgggtctggcaggcgaacgtggtccgcagcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaggaaattaaagaaaataccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggt

SEQ ID NO: 75 - DNA, 609 bp, Artificial Sequence
  synthesized, encoding fusion protein comprising: a fragment of TRAIL protein, peptide BAMP-28 and cleavage site sequences recognized by urokinase and metalloprotease.
ggtggtctgcgtagcctgggtcgtaaaattctgcgtgcatggaaaaaatatggtccgattattgtgccgattattcgtattcgtgttgttcgtccgctgggtctggcaggcgaacgtggtccgcagcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaggaaattaaagaaaataccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggt

SEQ ID NO: 76 - DNA, 600 bp, Artificial Sequence
  synthesized,encoding fusion protein comprising: a fragment of TRAIL protein, analogue of isoform C of the lytic peptide from Entamoeba histolytica, steric linker and cleavage site sequences recognized by urokinase and metalloprotease.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataagcagatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtagcggtggtccgctgggtctggcaggtcgtgttgttcgtggtctggttgaaaccctgaccaaaattgttagctatggtattgataaactgattgaaaaaattctggaaggt

SEQ ID NO: 77 - DNA, 600 bp, Artificial Sequence
  synthesized, encoding fusion protein comprising: a fragment of TRAIL protein, analogue of isoform A of the lytic peptide from Entamoeba histolytica, steric linker and cleavage site sequences recognized by urokinase and metalloprotease.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataagcagatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtagcggtggtccgctgggtctggcaggtcgtgttgttcgtggttttattgcaaccctgaccaaagttctggattttggtattgataaactgattcagctgattgaagataaa

SEQ ID NO: 78 - DNA, 600 bp, Artificial Sequence
  synthesized, encoding fusion protein comprising: a fragment of TRAIL protein, analogue of isoform B of the lytic peptide from Entamoeba histolytica, steric linker and cleavage site sequences recognized by urokinase and metalloprotease.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataagcagatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtagcggtggtccgctgggtctggcaggtcgtgttgttcgtggttttctgggcaccctggaaaaaattctgagctttggtgttgatgaactggttaaactgattgaaaatcat

SEQ ID NO: 79 - DNA, 570 bp, Artificial Sequence
  synthesized, encoding fusion protein comprising: a fragment of TRAIL protein, fragment of HA2 domain of influenza virus haemagglutinin, arginine transporting sequence and cleavage site sequences recognized by urokinase and metalloprotease.
ggtctgctggaagcactggcagaactgctggaaggtcggcgtcgtcgtcgtcggcgtcgtgttgttcgtccgctgggtctggcaggtcgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaacaccaaaaacgataaacaaatggtgcagtatatctataaatacaccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggt

SEQ ID NO: 80 - DNA, 1287 bp, Artificial Sequence
  synthesized, encoding fusion protein comprising: a fragment of TRAIL protein, a domain of alpha-toxin from Clostridium perfringens, sequences of steric linkers and pegylation linker.
tgggatggtaaaattgatggcaccggcacccatgcaatgattgttacccagggtgttagcattctggaaaatgatatgagcaaaaatgaaccggaaagcgtgcgtaaaaatctggaaattctgaaagataatatgcatgaactgcagctgggtagcacctatccggattatgataaaaatgcctatgatctgtatcaggatcatttttgggatccggataccaataataattttagcaaagataatagctggtatctggcctatagcattccggataccggtgaaagccagattcgtaaatttagcgcactggcacgttatgaatggcagcgtggtaattataaacaggcaaccttttatctgggcgaagccatgcattattttggtgatattgataccccgtatcatccggcaaatgttaccgcagttgatagcgcaggtcatgttaaatttgaaacctttgccgaagaacgcaaagaacagtataaaattaataccgtgggctgcaaaaccaatgaagatttttatgccgatatcctgaaaaataaagattttaatgcctggtccaaagaatatgcacgtggttttgcaaaaaccggcaaaagcatttattatagccatgcaagcatgagccatagctgggatgattgggattatgcagcaaaagttaccctggcaaatagccagaaaggcaccgcaggttatatttatcgttttctgcatgatgtgagcgaaggtaatggtggtggtggcggtggtagcgcaagcggttgtggtccggaaggcggtggtggtggtggttcacgtgttgcagcacatattaccggcacccgtggtcgtagcaataccctgagcagcccgaatagtaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaagaagaaattaaagaaaataccaaaaatgataagcagatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggt

SEQ ID NO: 81 - DNA, 1974 bp, Artificial Sequence
  synthesized, encoding fusion protein comprising: a fragment of TRAIL protein, listeriolysin O peptide, sequences of steric linkers, furin cleavage site and pegylation linker.
catgccgatgaaattgataaatatattcagggcctggattataataaaaacaatgtgctggtgtatcatggtgatgccgttaccaatgttccaccgcgtaaaggttataaagatggcaatgaatatattgttgtggagaaaaaaaaaaaatccatcaatcagaataatgccgatattcaggtggtgaatgcaattagcagcctgacctatccgggtgcactggttaaagcaaatagcgaactggttgaaaatcagccggatgttctgccggttaaacgtgatagcctgaccctgagcattgatctgcctggtatgaccaatcaggataataaaattgtggtgaaaaatgccaccaaaagcaatgttaataatgccgttaataccctggtggaacgctggaatgagaaatatgcacaggcatatccgaatgtgagcgccaaaattgattatgatgatgaaatggcctatagcgaaagccagctgattgcaaaatttggcaccgcatttaaagccgttaataatagcctgaatgtgaattttggtgccattagcgaaggcaaaatgcaggaagaagttattagctttaagcagatctattataacgtgaatgtgaatgaaccgacccgtccgagccgtttttttggtaaagcagttaccaaagaacagctgcaggcactgggtgttaatgcagaaaatcctccggcatatatttcaagcgttgcctatggtcgtcaggtttatctgaaactgagcaccaatagccatagcaccaaagttaaagcagcatttgatgcagcagttagcggtaaaagcgttagcggtgatgttgaactgaccaatattattaaaaattccagctttaaagccgtgatttatggtggtagcgccaaagatgaagtgcagattattgatggtaatctgggtgatctgcgcgatattctgaaaaaaggtgcaacctttaaccgtgaaacaccgggtgttccgattgcatataccaccaattttctgaaagataatgaactggccgtgattaaaaataatagcgaatatattgaaaccacgagcaaagcatataccgatggcaaaattaatattgatcatagcggtggctatgtggcccagtttaatattagctgggatgaaattaattatgatccggaaggcaatgaaattgtgcagcataaaaattggagcgaaaataataaaagcaaactggcccattttaccagcagcatttatctgccgggtaatgcacgtaatattaatgtgtatgccaaagaatgtaccggtctggcatgggaatggtggcgtaccgttattgatgatcgtaatctgccgctggtgaaaaatcgcaatattagcatttggggcaccaccctgtatccgaaatatagcaatagcgttggtggtggtggtagcggtggtggtggcggttcacgtaaaaaacgtgcaagcggttgtggtccggaaggtggcggtggcggtggtagccgtgttgcagcacatattaccggcacccgtggtcgtagcaataccctgagcagcccgaatagcaaaaatgaaaaagcactgggtcgtaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataagcagatggtgcagtatatctataaatataccagctatccggatccgattctgctgatgaaaagcgcacgtaatagctgttggagcaaagatgcagaatatggtctgtatagcatttatcagggtggcatctttgaactgaaagaaaatgatcgcatttttgtgagcgtgaccaatgaacatctgattgatatggatcatgaagccagcttttttggtgcatttctggttggt

SEQ ID NO: 82 - DNA, 1434 bp, Artificial Sequence
  synthesized, encoding fusion protein comprising: a fragment of TRAIL protein, phospholipase PC-PLC peptide, sequences of steric linkers, furin cleavage site and pegylation linker.
aaatttaaaaaagtggtgctgggtatgtgtctgaccgcaagcgttctggtttttccggttaccattaaagcaagcgcatgctgtgatgaatatctgaaacctccggcagcaccgcatgatattgatagcaaactgccgcataaactgagctggtcagcagataatccgaccaataccgatgtgaatacccattattggctgttcaaacaggccgaaaaaattctggccaaagatgttgatcacatgcgtgcaaatctgatgaatgaactgaaaaattttgataagcagattgcccagggcatttatgatgccgatcataaaaatccgtattatgataccagcacctttctgagccatttttataatccggataaagataatacctatctgccaggttttgccaatgcaaaaattaccggtgccaaatattttaatcagagcgttgccgattatcgcgaaggtaaatttgataccgccttttataaactgggcctggccattcattattataccgatattagccagccgatgcatgccaataattttaccgcaattagctatcctccgggttatcattgtgcctatgaaaattatgtggataccattaaacataattatcaggccaccgaagatatggttgttcagcgtttttgcagcaatgatgttaaagaatggctgtatgaaaatgccaaacgtgcaaaagccgattatccgaaaattgttaatgccaaaaccaaaaaaagctatctggtgggtaatagcgaatggaaaaaagataccgttgaaccgaccggtgcacgtctgcgtgatagccagcagaccctggcaggttttctggaattttggagcaaaaaaaccaatgaaggtggtggtggttctggtggtggtggcggtagccgtaaaaaacgtgcaagcggttgtggtccggaaggtggcggtggcggtggtagccgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaatcaaagaaaataccaaaaacgataagcagatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggt

SEQ ID NO: 83 - DNA, 1083 bp, Artificial Sequence
  synthesized, encoding fusion protein comprising: a fragment of TRAIL protein, equinatoxin EqTx-II, sequences of steric linkers, furin cleavage site and pegylation linker.
agcgcagatgttgccggtgcagttattgatggtgcaagcctgagctttgatattctgaaaaccgttctggaagccctgggtaatgtgaaacgtaaaattgcagtgggcgtggataatgaaagcggtaaaacctggaccgcactgaatacctattttcgtagcggcaccagcgatattgttctgccgcataaagttccgcatggtaaagcactgctgtataatggtcagaaagatcgtggtccggttgccaccggtgccgttggtgttctggcatatctgatgagtgatggtaataccctggcagttctgtttagcgttccgtatgattataactggtatagcaattggtggaacgtgcgtatctataaaggtaaacgtcgtgcagatcagcgcatgtatgaagaactgtattataacctgagcccgtttcgtggcgataatggttggcatacccgtaatctgggttatggtctgaaaagccgtggttttatgaatagcagcggtcatgcaattctggaaattcatgttaccaaagccggtggtggtggtagccgtaaaaaacgtgcaagcggttgtggtccggaaggtggtggcggttcacgtgttgcagcacatattaccggcacccgtggtcgtagcaataccctgagtagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttctactatatctatagccagacctatttccgcttccaagaagaaatcaaagaaaataccaaaaacgataaacaaatggtgcagtatatctacaaatacaccagctatccggatccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggtgggt

SEQ ID NO: 84 - DNA, 681 bp, Artificial Sequence
  synthesized, encoding fusion protein comprising: a fragment of TRAIL protein, viscotoxin A3 peptide, a sequence of steric linker and urokinase and metalloprotease cleavage sites.
aaaagctgttgtccgaataccaccggtcgcaatatttataatgcctgtcgtctgaccggtgcacctcgtccgacctgtgcaaaactgagcggctgcaaaattattagcggtagcacctgtccgagcgattatccgaaaccgctgggtctggcaggtcgtgttgttcgtggtggtagcggtggtgaacgtggtccgcagcgtgttgcagcacatattaccggcacccgtggtcgtagcaataccctgagcagcccgaatagcaaaaatgaaaaagccctgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataagcagatggtgcagtatatctataagtatacaagctatccggatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacatctgattgatatggatcatgaagccagcttttttggtgcatttctggtgggc

SEQ ID NO: 85 - DNA, 672 bp, Artificial Sequence
  synthesized, encoding fusion protein comprising: a fragment of TRAIL protein, viscotoxin A3 peptide, a sequence of steric linker and urokinase and metalloprotease cleavage sites.
cgtgttgcagcacatattaccggcacccgtggtcgtagcaataccctgagcagcccgaatagcaaaaatgaaaaagcactgggtcgcaaaattaatagctgggaaagcagccgtagcggtcatagctttctgagcaatctgcatctgcgtaatggtgaactggtgattcatgaaaaaggcttttattatatttatagccagacctattttcgctttcaggaagaaattaaagaaaataccaaaaatgataaacaaatggtgcagtatatctataaatataccagctatccggatccgattctgctgatgaaaagcgcacgtaatagctgttggagcaaagatgcagaatatggtctgtatagcatttatcagggtggcatttttgaactgaaagaaaatgatcgcatttttgtgagcgtgaccaatgaacatctgattgatatggatcatgaagccagcttttttggtgcatttctggttggtggtggtggtagcggtggtggtccgctgggtctggcaggtcgtgttgttcgtaaaagctgttgtccgaataccaccggtcgcaatatttataatgcatgtcgtctgaccggtgcacctcgtccgacctgtgcaaaactgagcggttgtaaaattattagcggtagcacctgtccgagcgattatccgaaa

SEQ ID NO: 86 - DNA, 600 bp, Artificial Sequence
  synthesized, encoding fusion protein comprising: a fragment of TRAIL protein, human perforin peptide and a sequence of steric linker.
cgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgataaacaaatggtgcagtacatctataaatataccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggtggtggtggcggtagcggagcaccgtgtcataccgcagcacgtagcgaatgtaaacgtagccataaatttgttccgggtgcatggctggcaggcgaaggtgttgatgttaccagcctg

SEQ ID NO: 87 - DNA, 630 bp, Artificial Sequence
  synthesized, encoding fusion protein comprising: a fragment of TRAIL protein, human perforin peptide, cleavage sites recognized by urokinase and metalloproteinase and a steric linker.
cgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttttattatatttatagccagacctattttcgctttcaagaagagattaaagaaaataccaaaaatgataaacaaatggtgcagtacatctataaatataccagctatccggacccgattctgctgatgaaaagcgcacgtaatagctgttggagcaaagatgcagaatatggtctgtatagcatttatcagggtggcatctttgagctgaaagaaaatgatcgcatctttgttagcgtgaccaacgaacatctgatcgatatggatcatgaagccagcttttttggtgcatttctggttggtggtggtggcggtagcggtcgtgttgttcgtccgctgggtctggctggcgcaccgtgtcataccgcagcacgtagcgaatgtaaacgtagccataaatttgttccgggtgcatggctggcaggcgaaggtgttgatgttaccagcctg

SEQ ID NO: 88 - DNA, 1308 bp, Artificial Sequence
  synthesized,encoding fusion protein comprising: a fragment of TRAIL protein, parasporin-2 from Bacillus thuringensis, cleavage sites recognized by urokinase and metalloproteinase and a steric linker.
gatgtgattcgcgaatatctgatgtttaatgaactgagcgcactgagcagcagtccggaaagcgttcgtagccgttttagcagcatttatggcaccaatccggatggtattgcactgaataatgaaacctatttcaatgccgtgaaacctccgattaccgcacagtatggttattattgctacaaaaatgttggcaccgtgcagtatgttaatcgtccgaccgatattaatccgaatgttattctggcacaggataccctgaccaataataccaatgaaccgtttaccaccaccattaccattaccggtagctttaccaataccagcaccgttaccagcagcaccaccaccggtttcaaatttaccagcaaactgagcatcaaaaaagtgtttgaaattggtggcgaagtgagctttagcaccaccattggcaccagcgaaaccaccaccgaaaccattaccgtgagcaaaagcgttaccgttaccgttccggcacagagccgtcgtaccattcagctgaccgcaaaaattgcaaaagaaagcgcagattttagcgcaccgattaccgttgatggttattttggtgcaaattttccgaaacgtgttggtccgggtggtcattacttttggtttaatccggcacgtgatgtgctgaataccaccagtggcaccctgcgtggtacagttaccaatgtttctagctttgattttcagaccattgttcagcctgcacgtagcctgctggatgaacagcgtgttgttcgtccgctgggtctggcaggcggtagcggtggtggttcaggtggtggtgaacgtggtccgcagcgtgttgcagcacatattaccggcacccgtggtcgtagcaataccctgagcagcccgaatagcaaaaatgaaaaagcactgggtcgcaaaatcaatagctgggaaagcagccgtagcggtcatagctttctgagcaatctgcatctgcgtaatggtgaactggtgattcatgaaaaaggcttctactatatttacagccagacctattttcgctttcaggaagaaattaaagaaaataccaaaaatgataaacaaatggtgcagtatatctataaatacaccagctatccggatccgattctgctgatgaaaagcgcacgtaatagctgttggagcaaagatgcagaatatggcctgtatagcatttatcagggtggcatttttgaactgaaagaaaatgatcgcatttttgtgagcgtgaccaatgaacatctgattgatatggatcatgaagcaagtttctttggtgcatttctggtgggc

SEQ ID NO: 89 - DNA, 645 bp, Artificial Sequence
  synthesized, encoding fusion protein comprising: a fragment of TRAIL protein, a fusion peptide comprising EGF inhibitor and synthetic lytic peptide, cleavage sites recognized by urokinase and metalloproteinase and steric linkers.
cgtgtggcggcgcatattaccggcacccgtggccgtagcaacaccctgagcagcccgaacagcaaaaacgaaaaagcgctgggccgtaaaattaacagctgggaaagcagccgtagcggccatagctttctgagcaacctgcatctgcgtaacggcgaactggtgattcatgaaaaaggcttttattatatttatagccagacctattttcgttttcaggaagaaattaaagaaaacaccaaaaacgataaacagatggtgcagtatatttataaatataccagctatccggatccgattctgctgatgaaaagcgcgcgtaacagctgctggagcaaagatgcggaatatggcctgtatagcatttatcagggcggcatttttgaactgaaagaaaacgatcgtatttttgtgagcgtgaccaacgaacatctgattgatatggatcatgaagcgagcttttttggcgcgtttctggtgggcggctgcgcggcgtgcgcggcggcgtgcggcggcggcccgctgggcctggcgggccgtgtggtgcgttataaatggtatggctataccccgcagaacgtgattggcggcggcaaactgctgctgaaactgctgaaaaaactgctgaaactgctgaaaaaaaaa

SEQ ID NO: 90 - protein, 281 aa, Homo sapiens [sequence omitted]

SEQ ID NO: 91 - protein, 223 aa, Artificial Sequence [sequence omitted]

SEQ ID NO: 92 - protein, 223 aa, Artificial Sequence
  synthesized, fusion protein comprising: a fragment of TRAIL protein, a synthetic fusion peptide, cleavage site recognized by furin, steric linkers and arginine transporting domain.
KLAKLAKKLAKLAKRRRRRRRRKKRGGGCAAACAACTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 93 - protein, 232 aa, Artificial Sequence
  synthesized, fusion protein comprising: a fragment of TRAIL protein, a synthetic fusion peptide, cleavage sites recognized by furin, steric linkers and arginine transporting domain.
KLAKLAKKLAKLAKRRRRRRRRKKRHRQPRGWEQGGGCAAACAACTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 94 - protein, 207 aa, Artificial Sequence
  synthesized, fusion protein comprising: a fragment of TRAIL protein, a synthetic fusion peptide, cleavage sites recognized by metalloproteinase and urokinase, steric linkers and transporting domain.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGPLGLAGRVVRYARAAARQARAGGKLAKLAKKLAKLAK

SEQ ID NO: 95 - protein, 218 aa, Artificial Sequence
  synthesized, fusion protein comprising: a fragment of TRAIL protein, a pleurocidine analogue, cleavage sites recognized by metalloproteinase and urokinase, steric linkers and transporting domain.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGPLGLAGRVVRYARAAARQARAGGRWGKWFKKATHVGKHVGKAALTAYL

SEQ ID NO: 96 - protein, 219 aa, Artificial Sequence
  synthesized, fusion protein comprising: a fragment of TRAIL protein, a pleurocidine analogue, cleavage sites recognized by metalloproteinase and urokinase, steric linkers and transporting domain.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGPLGLAGRVVRYARAAARQARAGGGRRKRKWLRRIGKGVKIIGGAALDHL

SEQ ID NO: 97 - protein, 212 aa, Artificial Sequence
  synthesized, fusion protein comprising: a fragment of TRAIL protein, a synthetic lytic peptide, cleavage sites recognized by metalloproteinase and urokinase, steric linkers and transporting domain.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGPLGLAGRVVRTHRPPMWSPVWPGGGKLLLKLLKKLLKLLKKK

SEQ ID NO: 98 - protein, 207 aa, Artificial Sequence
  synthesized, fusion protein comprising: a fragment of TRAIL protein, a synthetic lytic peptide, cleavage sites recognized by metalloproteinase and urokinase, steric linkers and transporting domain.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGPLGLAGRVVRYGRKKRRQRRRGGKLAKLAKKLAKLAK

SEQ ID NO: 99 - protein, 211 aa, Artificial Sequence [sequence omitted]

SEQ ID NO: 100 - protein, 210 aa, Artificial Sequence
  synthesized, fusion protein comprising: a fragment of TRAIL protein, a synthetic lytic peptide, cleavage sites recognized by metalloproteinase and urokinase, a transporting domain and steric linkers.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGPLGLAGRVVRYGRKKRRQRRRGGRFKRFRKKFKKLFKKLS

SEQ ID NO: 101 - protein, 211 aa, Artificial Sequence
  synthesized, fusion protein comprising: a fragment of TRAIL protein, a synthetic lytic peptide, cleavage sites recognized by metalloproteinase and urokinase and steric linkers.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGPLGLAGRVVRGGLRSLGRKILRAWKKYGQRLGNQWAVGHLM

SEQ ID NO: 102 - protein, 234 aa, Artificial Sequence
  synthesized, fusion protein comprising: a fragment of TRAIL protein, a melittin peptide, cleavage sites recognized by metalloproteinase and urokinase and steric linkers.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGPLGLAGRVVRGGGIGARLKVLTTGLPRISWIKRKRQQGGGGSKLAKLAKKLAKLAKRRRRRRRR

SEQ ID NO: 103 - protein, 205 aa, Artificial Sequence
  synthesized, fusion protein comprising: a fragment of TRAIL protein, a melittin peptide, cleavage sites recognized by metalloproteinase and urokinase and steric linker.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGPLGLAGRVVRGIGARLKVLTTGLPRISWIKRKRQQ

SEQ ID NO: 104 - protein, 215 aa, Artificial Sequence
  synthesized, fusion protein comprising: a fragment of TRAIL protein, a melittin peptide, cleavage sites recognized by metalloproteinase and urokinase, polyarginine transporting domain and steric linkers.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGPLGLAGRVVRRRRRRRRRGGGIGARLKVLTTGLPRISWIKRKRQQ

SEQ ID NO: 105 - protein, 215 aa, Artificial Sequence
  synthesized, fusion protein comprising: a fragment of TRAIL protein, a melittin peptide, cleavage sites recognized by metalloproteinase and urokinase, polyarginine transporting domain and steric linkers.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGPLGLAGRVVRGGGIGARLKVLTTGLPRISWIKRKRQQRRRRRRRR

SEQ ID NO: 106 - protein, 203 aa, Artificial Sequence
  synthesized, fusion protein comprising: a fragment of TRAIL protein, a synthetic lytic peptide, cleavage sites recognized by metalloproteinase and urokinase, polyarginine transporting domain and steric linker.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGPLGLAGRVVRRRRRRRRRKLLLRLLKKLLRLLK

SEQ ID NO: 107 - protein, 208 aa, Artificial Sequence
  synthesized, fusion protein comprising: a fragment of TRAIL protein, a synthetic lytic peptide, cleavage sites recognized by metalloproteinase and urokinase, a transporting domain and steric linkers.
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGPLGLAGRVVRYGRKKRRQRRRGGKLLLRLLKKLLRLLK

SEQ ID NO: 108 - DNA, 669 bp, Artificial Sequence
  synthesized, encoding fusion protein comprising: a fragment of TRAIL protein, a fusion peptide comprising EGF inhibitor and synthetic lytic peptide, cleavage sites recognized by urokinase and metalloproteinase and steric linkers.
aaactgctgctgaaactgctgaaaaaactgctgaaactgctgaaaaaaaaaggtggtggttatggtcgtccgcgtcagagcggtaaaaaacgtaaacgcaaacgtctgaaaccgacccgtgttgttcgtccgctgggtctggcaggcggtggttgtgcagcagcatgtgcagcctgtagcggtggtcgtgttgcagcacatattaccggcacccgtggtcgtagcaataccctgagcagcccgaatagcaaaaatgaaaaagcactgggtcgcaaaattaacagctgggaaagcagccgtagtggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggt

SEQ ID NO: 109 - DNA, 669 bp, Artificial Sequence
  synthesized, encoding fusion protein comprising: a fragment of TRAIL protein, a synthetic fusion peptide, cleavage site recognized by furin, steric linkers and arginine transporting domain.
aaactggcaaaactggccaaaaaactggctaaactggcgaaacgtcgtcgtcgccgtcgtcggcgtaaaaaacgtggtggtggttgtgcagcagcatgtgcagcctgtaccagcgaagaaaccattagcaccgttcaagaaaaacagcagaatattagtccgctggttcgtgaacgtggtccgcagcgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagcgagcttttttggtgcatttctggtgggt

SEQ ID NO: 110 - DNA, 696 bp, Artificial Sequence
  synthesized, encoding fusion protein comprising: a fragment of TRAIL protein, a synthetic fusion peptide, cleavage sites recognized by furin, steric linkers and arginine transporting domain.
aaactggcaaaactggccaaaaaactggctaaactggcgaaacgtcgtcgtcgccgtcgtcggcgtaaaaaacgtcatcgtcagccacgtggttgggaacagggtggtggttgtgcagcagcatgtgcagcctgtaccagcgaagaaaccattagcaccgttcaagaaaaacagcagaatattagtccgctggttcgtgaacgtggtccgcagcgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagcgagcttttttggtgcatttctggtgggt

SEQ ID NO: 111 - DNA, 621 bp, Artificial Sequence
  synthesized, encoding fusion protein comprising: a fragment of TRAIL protein, a synthetic fusion peptide, cleavage sites recognized by metalloproteinase and urokinase, steric linkers and transporting domain.
cgtgtggcggcgcatattaccggcacccgtggccgtagcaacaccctgagcagcccgaacagcaaaaacgaaaaagcgctgggccgtaaaattaacagctgggaaagcagccgtagcggccatagctttctgagcaacctgcatctgcgtaacggcgaactggtgattcatgaaaaaggcttttattatatttatagccagacctattttcgttttcaggaagaaattaaagaaaacaccaaaaacgataaacagatggtgcagtatatttataaatataccagctatccggatccgattctgctgatgaaaagcgcgcgtaacagctgctggagcaaagatgcggaatatggcctgtatagcatttatcagggcggcatttttgaactgaaagaaaacgatcgtatttttgtgagcgtgaccaacgaacatctgattgatatggatcatgaagcgagcttttttggcgcgtttctggtgggcggcggcggcggcagcggcggcggcggcccgctgggcctggcgggccgtgtggtgcgttatgcgcgtgcggcggcgcgtcaggcgcgtgcgggcggcaaactggcgaaactggcgaaaaaactggcgaaactggcgaaa

SEQ ID NO: 112 - DNA, 654 bp, Artificial Sequence
  synthesized, encoding fusion protein comprising: a fragment of TRAIL protein, a pleurocidine analogue, cleavage sites recognized by metalloproteinase and urokinase, steric linkers and transporting domain.
cgtgtggcggcgcatattaccggcacccgtggccgtagcaacaccctgagcagcccgaacagcaaaaacgaaaaagcgctgggccgtaaaattaacagctgggaaagcagccgtagcggccatagctttctgagcaacctgcatctgcgtaacggcgaactggtgattcatgaaaaaggcttttattatatttatagccagacctattttcgttttcaggaagaaattaaagaaaacaccaaaaacgataaacagatggtgcagtatatttataaatataccagctatccggatccgattctgctgatgaaaagcgcgcgtaacagctgctggagcaaagatgcggaatatggcctgtatagcatttatcagggcggcatttttgaactgaaagaaaacgatcgtatttttgtgagcgtgaccaacgaacatctgattgatatggatcatgaagcgagcttttttggcgcgtttctggtgggcggcggcggcggcagcggcggcggcggcccgctgggcctggcgggccgtgtggtgcgttatgcgcgtgcggcggcgcgtcaggcgcgtgcgggcggccgttggggcaaatggtttaaaaaagcgacccatgtgggcaaacatgtgggcaaagcggcgctgaccgcgtatctg

SEQ ID NO: 113 - DNA, 657 bp, Artificial Sequence
  synthesized, encoding fusion protein comprising: a fragment of TRAIL protein, a pleurocidine analogue, cleavage sites recognized by metalloproteinase and urokinase, steric linkers and transporting domain.
cgtgtggcggcgcatattaccggcacccgtggccgtagcaacaccctgagcagcccgaacagcaaaaacgaaaaagcgctgggccgtaaaattaacagctgggaaagcagccgtagcggccatagctttctgagcaacctgcatctgcgtaacggcgaactggtgattcatgaaaaaggcttttattatatttatagccagacctattttcgttttcaggaagaaattaaagaaaacaccaaaaacgataaacagatggtgcagtatatttataaatataccagctatccggatccgattctgctgatgaaaagcgcgcgtaacagctgctggagcaaagatgcggaatatggcctgtatagcatttatcagggcggcatttttgaactgaaagaaaacgatcgtatttttgtgagcgtgaccaacgaacatctgattgatatggatcatgaagcgagcttttttggcgcgtttctggtgggcggcggcggcggcagcggcggcggcggcccgctgggcctggcgggccgtgtggtgcgttatgcgcgtgcggcggcgcgtcaggcgcgtgcgggcggcggccgtcgtaaacgtaaatggctgcgtcgtattggcaaaggcgtgaaaattattggcggcgcggcgctggatcatctg

SEQ ID NO: 114 - DNA, 636 bp, Artificial Sequence
  synthesized, encoding fusion protein comprising: a fragment of TRAIL protein, a synthetic lytic peptide, cleavage sites recognized by metalloproteinase and urokinase, steric linkers and transporting domain.
cgtgtggcggcgcatattaccggcacccgtggccgtagcaacaccctgagcagcccgaacagcaaaaacgaaaaagcgctgggccgtaaaattaacagctgggaaagcagccgtagcggccatagctttctgagcaacctgcatctgcgtaacggcgaactggtgattcatgaaaaaggcttttattatatttatagccagacctattttcgttttcaggaagaaattaaagaaaacaccaaaaacgataaacagatggtgcagtatatttataaatataccagctatccggatccgattctgctgatgaaaagcgcgcgtaacagctgctggagcaaagatgcggaatatggcctgtatagcatttatcagggcggcatttttgaactgaaagaaaacgatcgtatttttgtgagcgtgaccaacgaacatctgattgatatggatcatgaagcgagcttttttggcgcgtttctggtgggcggcggcggcggcagcggcggcggcggcccgctgggcctggcgggccgtgtggtgcgtacccatcgtccgccgatgtggagcccggtgtggccgggcggcggcaaactgctgctgaaactgctgaaaaaactgctgaaactgctgaaaaaaaaa

SEQ ID NO: 115 - DNA, 621 bp, Artificial Sequence
  synthesized, encoding fusion protein comprising: a fragment of TRAIL protein, a synthetic lytic peptide, cleavage sites recognized by metalloproteinase and urokinase, steric linkers and transporting domain.
cgcgtggcggcgcatattaccggcacccgcggccgcagcaacaccctgagcagcccgaacagcaaaaacgaaaaagcgctgggccgcaaaattaacagctgggaaagcagccgcagcggccatagctttctgagcaacctgcatctgcgcaacggcgaactggtgattcatgaaaaaggcttttattatatttatagccagacctattttcgctttcaggaagaaattaaagaaaacaccaaaaacgataaacagatggtgcagtatatttataaatataccagctatccggatccgattctgctgatgaaaagcgcgcgcaacagctgctggagcaaagatgcggaatatggcctgtatagcatttatcagggcggcatttttgaactgaaagaaaacgatcgcatttttgtgagcgtgaccaacgaacatctgattgatatggatcatgaagcgagcttttttggcgcgtttctggtgggcggcggcggcggcagcggcggcggcggcccgctgggcctggcgggccgcgtggtgcgctatggccgcaaaaaacgccgccagcgccgccgcggcggcaaactggcgaaactggcgaaaaaactggcgaaactggcgaaa

SEQ ID NO: 116 - DNA, 633 bp, Artificial Sequence
  synthesized,encoding fusion protein comprising: a fragment of TRAIL protein, a synthetic lytic peptide, cleavage sites recognized by metalloproteinase and urokinase and a steric linker.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggcggtagtggcggtggtggtcctctgggtctggcaggtcgtgttgttcgtggtcgttttaaacgttttcgcaaaaaatttaaaaaactgttcaaaaaactgagccagcgcctgggtaatcagtgggcagttggtcatctgatg

SEQ ID NO: 117 - DNA, 630 bp, Artificial Sequence
  synthesized,encoding fusion protein comprising: a fragment of TRAIL protein, a synthetic lytic peptide, cleavage sites recognized by metalloproteinase and urokinase, a transporting domain and steric linkers.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggcggtagtggcggtggtggtcctctgggtctggcaggtcgtgttgttcgttatggtcgtaaaaaacgtcgtcagcgtcgtcgtggtggtcgttttaaacgttttcgcaaaaaatttaaaaaactgttcaaaaaactgagc

SEQ ID NO: 118 - DNA, 633 bp, Artificial Sequence
  synthesized, encoding fusion protein comprising: a fragment of TRAIL protein, a synthetic lytic peptide, cleavage sites recognized by metalloproteinase and urokinase and steric linkers.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggcggtagtggcggtggtggtcctctgggtctggcaggtcgtgttgttcgtggtggtctgcgtagcctgggacgtaaaattctgcgtgcatggaagaaatatggtcagcgtctgggtaatcagtgggcagttggtcatctgatg

SEQ ID NO: 119 - DNA, 702 bp, Artificial Sequence
  synthesized, encoding fusion protein comprising: a fragment of TRAIL protein, a melittin peptide, cleavage sites recognized by metalloproteinase and urokinase and steric linkers.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggcggtagtggcggtggtggtcctctgggtctggcaggtcgtgttgttcgcggaggtggtattggtgcacgtctgaaagttctgaccaccggtctgcctcgtattagctggattaaacgtaaacgtcagcagggtgggggtggtagcaaactggcaaaactggcgaaaaaactggctaaactggccaaacgtcgtcgtcgccgtcgtcggcgt

SEQ ID NO: 120 - DNA, 615 bp, Artificial Sequence
  synthesized, encoding fusion protein comprising: a fragment of TRAIL protein, a melittin peptide, cleavage sites recognized by metalloproteinase and urokinase and steric linker.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggcggtagtggcggtggtggtcctctgggtctggcaggtcgtgttgttcgtggtattggtgcacgtctgaaagttctgaccaccggtctgcctcgtattagctggattaaacgtaaacgtcagcag

SEQ ID NO: 121 - DNA, 645 bp, Artificial Sequence
  synthesized,encoding fusion protein comprising: a fragment of TRAIL protein, a melittin peptide, cleavage sites recognized by metalloproteinase and urokinase, polyarginine transporting domain and steric linkers.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggcggtagtggcggtggtggtcctctgggtctggcaggtcgtgttgttcgtcgtcgtcgccgtcggcgtcgtcgcggtggtggaattggtgcacgtctgaaagttctgaccaccggtctgcctcgtattagctggattaaacgtaaacgtcagcag

SEQ ID NO: 122 - DNA, 645 bp, Artificial Sequence
  synthesized,encoding fusion protein comprising: a fragment of TRAIL protein, a melittin peptide, cleavage sites recognized by metalloproteinase and urokinase, polyarginine transporting domain and steric linkers.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggcggtagtggcggtggtggtcctctgggtctggcaggtcgtgttgttcgcggaggtggtattggtgcacgtctgaaagttctgaccaccggtctgcctcgtattagctggattaaacgtaaacgtcagcagcgtcgtcgtcgccgtcgtcggcgt

SEQ ID NO: 123 - DNA, 609 bp, Artificial Sequence
  synthesized, encoding fusion protein comprising: a fragment of TRAIL protein, a synthetic lytic peptide, cleavage sites recognized by metalloproteinase and urokinase, polyarginine transporting domain and steric linker.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggcggtagtggcggtggtggtcctctgggtctggcaggtcgtgttgttcgtcgtcgtcgccgtcggcgtcgtcgtaaactgctgctgcgtctgctgaaaaaactgctgcgcctgctgaaa

SEQ ID NO: 124 - DNA, 624 bp, Artificial Sequence
  synthesized, encoding fusion protein comprising: a fragment of TRAIL protein, a synthetic lytic peptide, cleavage sites recognized by metalloproteinase and urokinase, a transporting domain and steric linkers.
cgtgttgcagcacatattaccggcacccgtggtcgtagcaataccctgagcagcccgaatagcaaaaatgaaaaagcactgggtcgcaaaattaacagctgggaaagcagccgtagcggtcatagctttctgagcaatctgcatctgcgtaatggtgaactggtgattcatgaaaaaggcttctactatatctacagccagacctattttcgcttccaagaagagattaaagaaaacaccaaaaacgataaacaaatggtgcagtacatctataaatacaccagctatccggatccgattctgctgatgaaaagcgcacgtaatagctgttggagcaaagatgcagaatatggcctgtatagcatttatcagggtggcatctttgaactgaaagaaaacgatcgtattttcgtgagcgtgaccaatgaacatctgatcgatatggatcatgaagccagcttttttggtgcatttctggtgggtggtggtggcggtagtggcggtggtggtcctctgggtctggcaggtcgtgttgttcgttatggtcgtaaaaaacgtcgtcagcgtcgtcgtggtggtaaactgctgctgcgtctgctgaaaaaactgctgcgcctgctgaaa

SEQ ID NO: 125 - protein, 39 aa, Artificial Sequence
  synthesized efector peptide
KLLLKLLKKLLKLLKKKGGGYGRPRQSGKKRKRKRLKPT

SEQ ID NO: 126 - protein, 25 aa, Pleuronectes americanus
  ref <301>: AUTHORS: Hilchie AL, Doucette CD, Pinto DM, Patrzykat A, Douglas S, Hoskin DW.
  ref <302>: TITLE: Pleurocidin-family cationic antimicrobial peptides are cytolytic for breast carcinoma cells and prevent growth of tumor xenografts
  ref <303>: JOURNAL: Breast Cancer Res.
  ref <304>: VOLUME: 13
  ref <305>: ISSUE: 5
  ref <306>: PAGES: R102
  ref <307>: DATE: 2011-10-24
RWGKWFKKATHVGKHVGKAALTAYL

SEQ ID NO: 127 - protein, 26 aa, Pleuronectes americanus
  ref <301>: AUTHORS: Hilchie AL, Doucette CD, Pinto DM, Patrzykat A, Douglas S, Hoskin DW.
  ref <302>: TITLE: Pleurocidin-family cationic antimicrobial peptides are cytolytic for breast carcinoma cells and prevent growth of tumor xenografts.
  ref <303>: JOURNAL: Breast Cancer Res.
  ref <304>: VOLUME: 13
  ref <305>: ISSUE: 5
  ref <306>: PAGES: R102
  ref <307>: DATE: 2011-10-24
GRRKRKWLRRIGKGVKIIGGAALDHL

SEQ ID NO: 128 - protein, 17 aa, Artificial Sequence
  synthesized efector peptide
  ref <301>: AUTHORS: Papo N, Shai Y.
  ref <302>: TITLE: New lytic peptides based on the D,L-amphipathic helix motif preferentially kill tumor cells compared to normal cells.
  ref <303>: JOURNAL: Biochemistry
  ref <304>: VOLUME: 42
  ref <305>: ISSUE: 31
  ref <306>: PAGES: 9346-54
  ref <307>: DATE: 2003-08-12
KLLLKLLKKLLKLLKKK

SEQ ID NO: 129 - protein, 31 aa, Artificial Sequence
  synthesized efector peptide
  ref <301>: AUTHORS: Cai H, Yang H, Xiang B, Li S, Liu S, Wan L, Zhang J, Li Y, Cheng J, Lu X.
  ref <302>: TITLE: Selective apoptotic killing of solid and hematologic tumor cells by bombesin-targeted delivery of mitochondria-disrupting peptides.
  ref <303>: JOURNAL: Molecular Pharmacology
  ref <304>: VOLUME: 7
  ref <305>: ISSUE: 2
  ref <306>: PAGES: 586-96
  ref <307>: DATE: 2010-04-05
GRFKRFRKKFKKLFKKLSQRLGNQWAVGHLM

SEQ ID NO: 130 - protein, 17 aa, Artificial Sequence
  synthesized efector peptide
  ref <301>: AUTHORS: Cai H, Yang H, Xiang B, Li S, Liu S, Wan L, Zhang J, Li Y, Cheng J, Lu X.
  ref <302>: TITLE: Selective apoptotic killing of solid and hematologic tumor cells by bombesin-targeted delivery of mitochondria-disrupting peptides.
  ref <303>: JOURNAL: Molecular Pharmacology
  ref <304>: VOLUME: 7
  ref <305>: ISSUE: 2
  ref <306>: PAGES: 586-96
  ref <307>: DATE: 2010-04-05
RFKRFRKKFKKLFKKLS

SEQ ID NO: 131 - protein, 29 aa, Artificial Sequence
  synthesized efector peptide
  ref <301>: AUTHORS: Cai H, Yang H, Xiang B, Li S, Liu S, Wan L, Zhang J, Li Y, Cheng J, Lu X.
  ref <302>: TITLE: Selective apoptotic killing of solid and hematologic tumor cells by bombesin-targeted delivery of mitochondria-disrupting peptides.
  ref <303>: JOURNAL: Molecular Pharmacology
  ref <304>: VOLUME: 7
  ref <305>: ISSUE: 2
  ref <306>: PAGES: 586-96
  ref <307>: DATE: 2010-04-05
LRSLGRKILRAWKKYGQRLGNQWAVGHLM

SEQ ID NO: 132 - protein, 25 aa, Apis mellifera
  ref <308>: DATABASE ACCESSION NUMBER: genbank/AAU87881
  ref <309>: DATABASE ENTRY DATE: 2004-10-04
  ref <313>: RELEVANT RESIDUES IN SEQ ID NO: (1)..(25)
GIGARLKVLTTGLPRISWIKRKRQQ